(19) United States
(12) Patent Application Publication
(10) Pub. No.: US 2009/0062255 A1
(43) Pub. Date: Mar. 5, 2009

(54) TUMOR-TARGETING EVALUATION METHODOLOGY AND COMPOUNDS RELATED THERETO

(75) Inventors: Henriette GOURDEAU, Montreal (CA); Pierre FALARDEAU, Westmount (CA); My-Anh WIOLAND, Montreal (CA); Nadia BOUFAIED, Terrebonne (CA); James B. MCALPINE, Westmount (CA); Mustapha AOUIDATE, Laval (CA); Thierry BERTOMEU, Montreal (CA); Ashraf IBRAHIM, Kirkland (CA)

Correspondence Address: MERCHANT & GOULD PC, P.O. BOX 2903, MINNEAPOLIS, MN, 55402-0903, US

(73) Assignee: THALLION PHARMACEUTICALS INC., Montreal (CA)

(21) Appl. No.: 12/192,595

(22) Filed: Aug. 15, 2008

Related U.S. Application Data
(60) Provisional application No. 60/935,552, filed on Aug. 17, 2007.
(60) Provisional application No. 60/980,689, filed on Oct. 17, 2007.

(51) Int. Cl. A61K 31/551; C07D 487/00; A61P 31/00; G01N 33/574

(52) U.S. Cl. 514/212.06; 540/577; 435/7.23

(57) ABSTRACT

The invention relates to a method for evaluating a chemotherapeutic potential of a candidate molecule. In evaluating the candidate molecule, the candidate molecule is tested for its ability to inhibit the in vitro growth of a cancer cell; to bind a cellular receptor produced by a cancer cell, wherein said receptor, such as a peripheral benzodiazepine receptor, is produced in a greater amount by said cancer cell than by a normal cell; and to inhibit the activity of at least one protein member of the MAPK pathway. The invention further relates to dibenzodiazepinone analogues and derivatives thereof.

### RELATED APPLICATIONS

This application claims benefit under 35 USC §119 of Provisional Application U.S. Ser. No. 60/935,552, filed Aug. 17, 2007 and Provisional Application U.S. Ser. No. 60/980,689, filed Oct. 17, 2007, the entire teachings of which are herein incorporated by reference for all purposes.

### FIELD OF THE INVENTION

The present invention relates to dibenzodiazepinone analogues, including a naturally produced farnesylated dibenzodiazepinone referred to herein as Compound 1, and to chemical derivatives of the analogues, as well as to pharmaceutically acceptable salts, solvates and prodrugs of the analogues and derivatives, and to methods for obtaining these compounds.

The present invention also relates to compounds that exhibit one or more of the following characteristics: (a) peripheral benzodiazepine receptor (PBR) binding activity, (b) Ras-MAPK pathway inhibition, and (c) cytotoxic activity, as well as to methods for screening for compounds having one or more of these activities.

One method of obtaining Compound 1 is by cultivation of a strain of a Micromonospora sp., e.g., 046-ECO11 or [S01]046. One method of obtaining the derivatives involves post-biosynthesis chemical modification of Compound 1. The present invention further relates to the use of dibenzodiazepinone analogues, and their pharmaceutically acceptable salts, solvates and prodrugs as pharmaceuticals, in particular to their use as inhibitors of cancer cell growth, mammalian lipoxygenase, and for treating acute and chronic inflammation, and to pharmaceutical compositions comprising a dibenzodiazepinone analogue, or a pharmaceutically acceptable salt, solvate or prodrug thereof.

### BACKGROUND OF THE INVENTION

The euactinomycetes are a subset of a large and complex group of Gram-positive bacteria known as actinomycetes. Over the past few decades these organisms, which are abundant in soil, have generated significant commercial and scientific interest as a result of the large number of therapeutically useful compounds, particularly antibiotics, produced as secondary metabolites. The intensive search for strains able to produce new antibiotics has led to the identification of hundreds of new species.

Many of the euactinomycetes, particularly Streptomyces and the closely related Saccharopolyspora genera, have been extensively studied. Both of these genera produce a notable diversity of biologically active metabolites. Because of the commercial significance of these compounds, much is known about the genetics and physiology of these organisms.

Another representative genus of euactinomycetes, Micromonospora, has also generated commercial interest. For example, U.S. Pat. No. 5,541,181 (Ohkuma et al., 1996) discloses a dibenzodiazepinone compound, specifically 5-farnesyl-4,7,9-trihydroxy-dibenzodiazepin-11-one (named “BU-4664L”), produced by a known euactinomycetes strain, Micromonospora sp. M990-6 (ATCC 55378).

The drug discovery DECIPHER® platform of Thallion Pharmecuticals Inc. can predict chemical structures of potential new drugs by scanning gene clusters of microorganisms (Farnet and Zazopoulos (2005) in Natural Products: Drug Discovery and Therapeutic Medicine at pp 95-106; McAlpine et al (2005) J Nat Prod pp 68: 493-6; Zazopoulos et al (2003) Nat Biotechnol 21, pp 187-90).

ECO-4601 (4,6,8-trihydroxy-10-(3,7,11-trimethyldodeca-2,6,10-trienyl)-5,10-dihydrodibenzo[b,e][1,4]diazepin-11-one), a farnesylated dibenzodiazepinone (MW 462.58) is one of the natural compounds identified using DECIPHER® (Bachmann et al (2004) U.S. Pat. No. 7,101,872) to analyze actinomycete gene loci encoding pathways leading to bioactive compounds (McAlpine et al (2005) J Nat Prod 68: pp 493-6; Zazopoulos et al (2003) Nat Biotechnol 21: pp 187-90). The compound was also isolated and characterized by Wyeth Laboratories (Charan et al (2004) J Nat Prod 67, pp 1431-3). Initial in vitro assessment by the U.S. National Cancer Institute (NCI) showed that ECO-4601 had broad cytotoxic activity in the low micromolar range inhibiting the growth of hematological and solid tumor cell lines, and thus a good candidate for clinical studies against brain and other solid tumors.

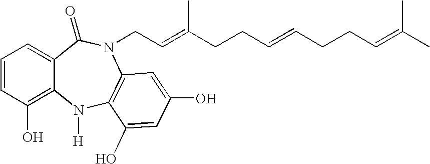

### ECO-4601 (Compound 1 of the Present Invention)

ECO-4601 and the novel Micromonospora sp. strains 046-ECO11 and [S01]046 that have been found to produce it, are disclosed in Canadian Patent No. 2,466,340.

As ECO-4601 was identified through in vitro cytotoxic assays, its molecular target(s) were unknown at the time of discovery. The strategy used to determine the mechanism of action of ECO-4601 was thus based on its chemical structure. Since ECO-4601 contains a benzodiazepine moiety, it was first determined whether the compound could bind the central (GABA_{A}; CBR) and/or peripheral (PBR) benzodiazepine receptors. The CBR is restricted to the central nervous system and mediates the anxiolytic and anticonvulsant properties of benzodiazepines (Olsen et al (1990) Faseb J 4 pp 1469-80; Stephenson (1995) Biochem J 310 (Part 1), pp 1-9).

On the other hand, the PBR was originally discovered as an alternative binding site for the benzodiazepine, diazepam (Valium®) (Braestrup C, Squires RF (1977) Specific benzodiazepine receptors in rat brain characterized by high-affinity (3H)diazepam binding. Proc Natl Acad Sci USA 74: 3805-9), and it is a critical component of the mitochondrial permeability transition pore (MPTP) (Decaudin (2004) Anticancer Drugs 15, pp 737-45; Galiegue et al (2003). Curr Med Chem 10 pp 1563-72; Papadopoulos (2003) Ann pharm Fr 61 (1), pp 30-50; Papadopoulos et al (2001) Therapie 56 pp 549-56; Papadopoulos et al (2006) Trends Pharmacol Sci 27, pp 402-9). This multiprotein complex is located at the contact site between inner and outer mitochondrial membranes and is involved in the initiation and regulation of apoptosis.

Although present in most tissues, the PBR is highly abundant in glandular and steroid-producing tissues such as adrenal glands and gonads (Bribes et al (2004) J Histochem Cytochem 52 pp 19-28; Casellas et al (2002) Neurochem Int 40 pp 475-86; Beurdeley-Thomas et al (2000) J Neurooncol 46 pp 45-56; Zisterer et al (1997) Gen Pharmacol 29 pp 305-14). Under neuroinflammatory conditions and in various cancers, the PBR is up-regulated and is considered as a specific marker for the visualization of neuropathologies as well as a prognostic factor for breast, colorectal and prostate tumors (Casellas et al (2002) Neurochem Int 40 pp 475-86; Han et al (2003) J Recept Signal Transduct Res 23 pp 225-38; Maaser et al (2002) Clin Cancer Res 8 pp 3205-9; Papadopoulos (2003) Ann pharm Fr 61 (1), pp 30-50).

PBRs are involved in steroidogenesis, heme biosynthesis, immune and stress responses, cell growth, differentiation, and mitochondrial respiratory control (Beurdeley-Thomas et al (2000) J Neurooncol 46: 45-56; Galiegue et al (2003) Curr Med Chem 10, pp 1563-72; Krueger (1995) Biochim Biophys Acta 1241, pp 453-70; Papadopoulos (2003) Ann pharm Fr 61 (1), pp 30-50). Increase in PBRs is documented in many tumor types compared to normal tissues (Han et al (2003) J Recept Signal Transduct Res 23, pp 225-38).

While PBR ligands have been reported to inhibit cell proliferation and to induce apoptosis, no clear antitumor activity resulting from their direct interaction with PBR per se has been observed (Decaudin et al (2002) Cancer Res 62 pp 1388-93; Hans et al (2005) Biochem Pharmacol 69, pp 819-30; Xia et al (2000) Proc Natl Acad Sci USA 97, pp 7494-9).

Binding of PBR-specific ligands, such as PK11195 (1-(2-chlorophenyl)-N-methyl-(1-methylpropyl)-3-isoquinoline carboxamide), is highly increased in several solid tumor types including colon (Katz et al (1990) Oncology 47, pp 139-42; Maaser et al (2002) Clin Cancer Res 8, pp 3205-9), brain (Cornu et al (1992) Acta Neurochir (Wien) 119, pp 146-52), breast (Beinlich et al (1999) Life Sci 65, pp 2099-108; Carmel et al (1999) Biochem Pharmacol 58, pp 273-8; Hardwick et al (1999) Cancer Res 59, pp 831-42), prostate (Hanet al (2003) J Recept Signal Transduct Res 23, pp 225-38), ovary (Batra et al (2000) Int J Mol Med 5, pp 619-23) and liver (Venturini et al (1999) Life Sci 65, pp 2223-31; Venturini et al (1998) Life Sci 63, pp 1269-80).

Moreover, PBR ligands have been used as imaging tools in the diagnosis of brain tumors (Broaddus et al (1990) Brain Res 518, pp 199-208; Maeda et al (2004) Synapse 52, pp 283-91) and have been shown to inhibit proliferation and induce apoptosis of rat C6 glioma cells (Chelli et al (2004) Biochem Pharmacol 68, pp 125-34).

Results of benzodiazepine receptor binding assays indicated that ECO-4601 had a selective affinity for the PBR (IC₅₀=0.291 μM), with no specific binding to the CBR (IC₅₀>10 μM). These data imply that other cellular targets must contribute to the potent antitumor activity of ECO-4601.

The Ras-MAPK signaling pathway controls cell growth, differentiation and survival. This signaling pathway has long been viewed as an attractive pathway for anticancer therapies, based on its central role in regulating the growth and survival of cells from a broad spectrum of human tumors, and mutations in components of this signaling pathway underlie tumour initiation in mammal cells (Sebolt-Leopold et al (2004) Nat Rev Cancer 4, pp 937-47).

The Ras-MAPK signaling pathway is activated by a variety of extracellular signals (hormones and growth factors), which activate Ras by exchanging GDP with GTP. Ras then recruits Raf to the plasma membrane where its activation takes place. As noted above, mutations in components of the signaling pathway, resulting in constitutive activation, underlie tumor initiation in mammalian cells. For example, growth factor receptors, such as epidermal growth factor receptor (EGFR), are subject to amplifications and mutations in many cancers, accounting for up to 25% of non-small cell lung cancers and 60% of glioblastomas. Braf is also frequently mutated, particularly in melanomas (approximately 70% of cases) and colon carcinomas (approximately 15% of cases). Moreover, Ras is the most frequently mutated oncogene, occurring in approximately 30% of all human cancers. The frequency and type of mutated Ras genes (H-Ras, K-Ras or N-Ras) varies widely depending on the tumor type. K-Ras is, however, the most frequently mutated gene, with the highest incidence detected in oancreatic cancer (approximately 90%) and colorectal cancer (approximately 45%). This makes it, as well as other components of the signaling pathway, an appropriate target for anticancer therapy. Indeed, small-molecular weight inhibitors designed to target various steps of this pathway have entered clinical trials. Moreover, sorafenib (Nexavar®, Bayer HealthCare Pharmaceuticals), a Raf-kinase inhibitor resulting in Ras signaling inhibition, has recently been approved against renal cell carcinoma. Following these data, there continues to be a high level of interest in targeting the Ras-MAPK pathway for the development of improved cancer therapies.

Although many biologically active compounds have been identified from bacteria, there remains the need to obtain novel compounds with enhanced properties such as having an ability to bind to the PBR and to effect signaling in the Ras-MAPK pathway. As well, there exists a considerable need to obtain pharmaceutically active compounds in a cost-effective manner and with high yield. The present invention addresses these problems providing novel methods for screening for these compounds and further provides novel compounds having therapeutic potential and methods to generate these novel compounds by post-biosynthetic chemical modifications.

### SUMMARY OF THE INVENTION

The present invention is directed to dibenzodiazepinone analogues, which includes each of Compounds 1-100, as well as each of the compounds defined by Formula I and Formula II, and to pharmaceutically acceptable salt, solvate or prodrug of each of these compounds.

The present invention is also directed to derivatives of the dibenzodiazepinone analogues, which includes derivatives of Compounds 1-100, derivatives of the compounds defined by Formula I and Formula II, and pharmaceutically acceptable salts, solvates and prodrugs thereof. In preferred embodiments, the derivatives include ethers, esters, N-alkylated derivatives and N-acylated derivatives of the dibenzodiazepinone analogues of the present invention.

The present invention is additionally directed to pharmaceutical compositions comprising one or more of: (a) the dibenzodiazepinone analogues of the present invention, and (b) the dibenzodiazepinone analogue derivatives of the present invention, together with a pharmaceutically acceptable carrier.

In particular, Compounds 1-100 are as follows:

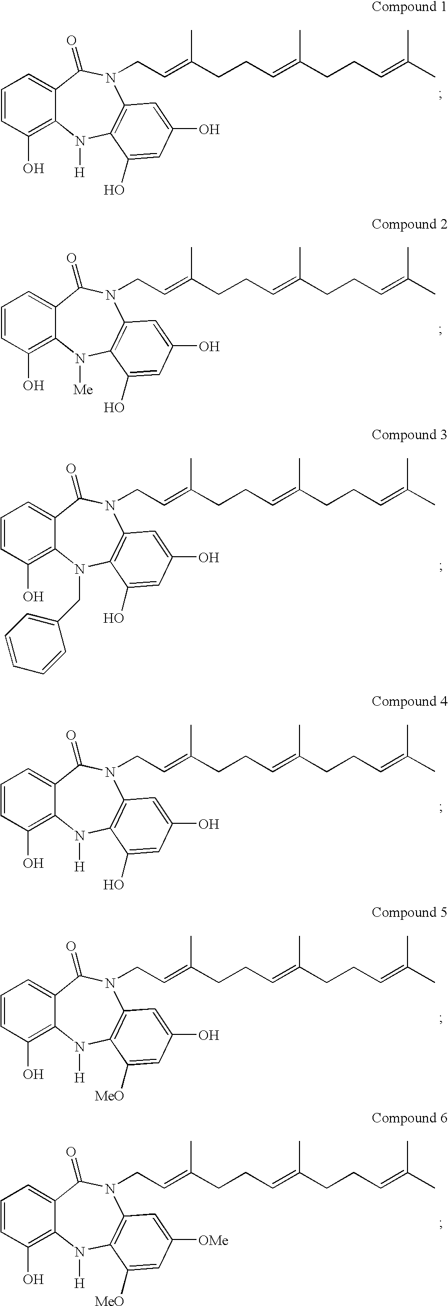

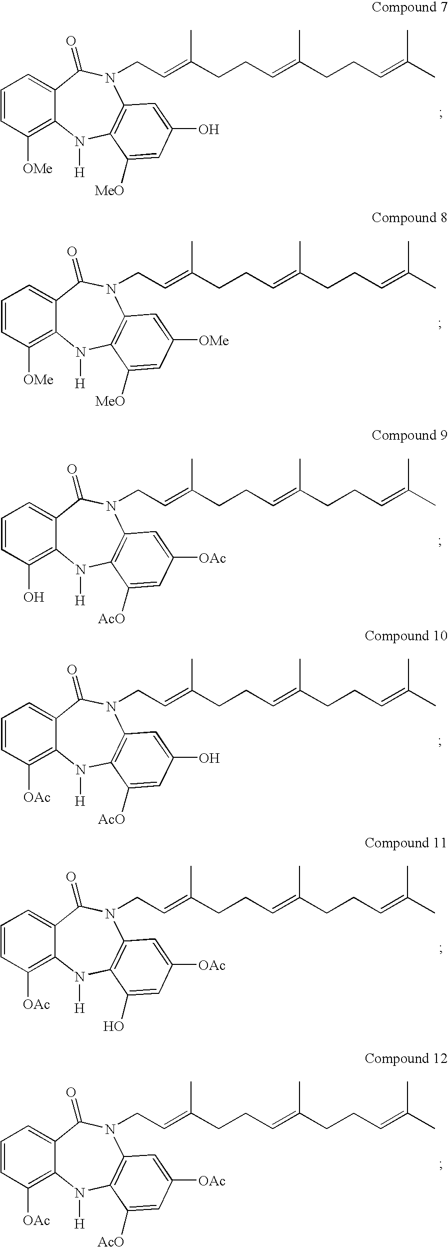

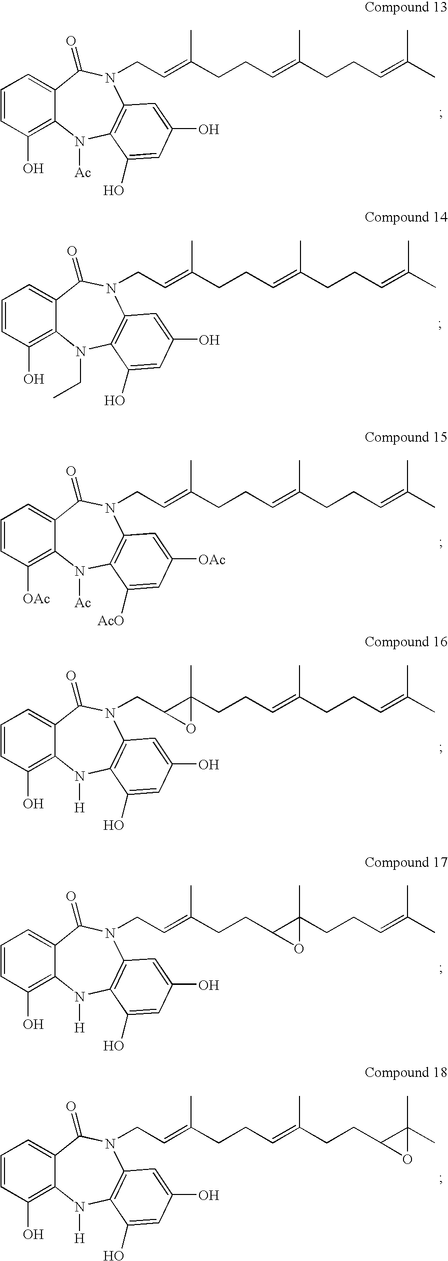

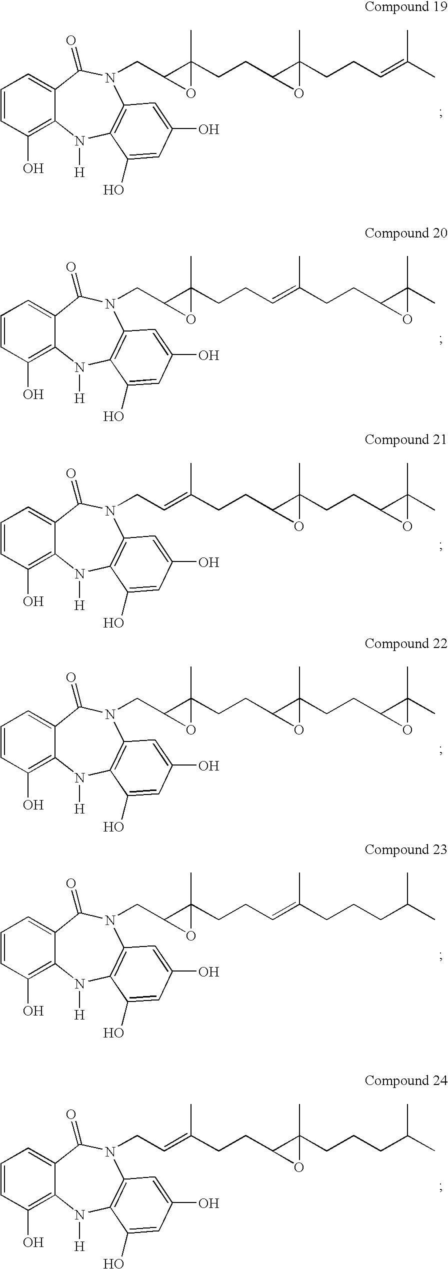

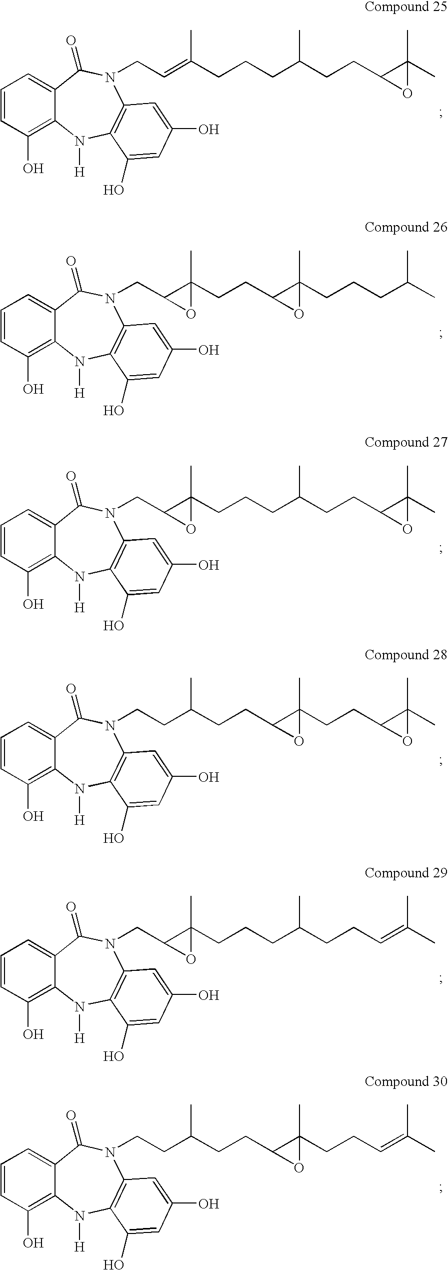

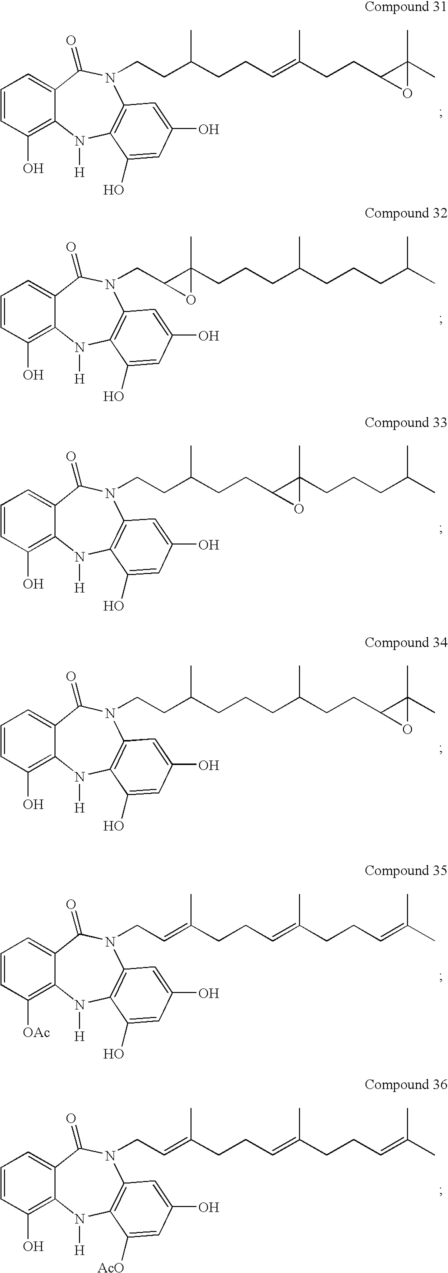

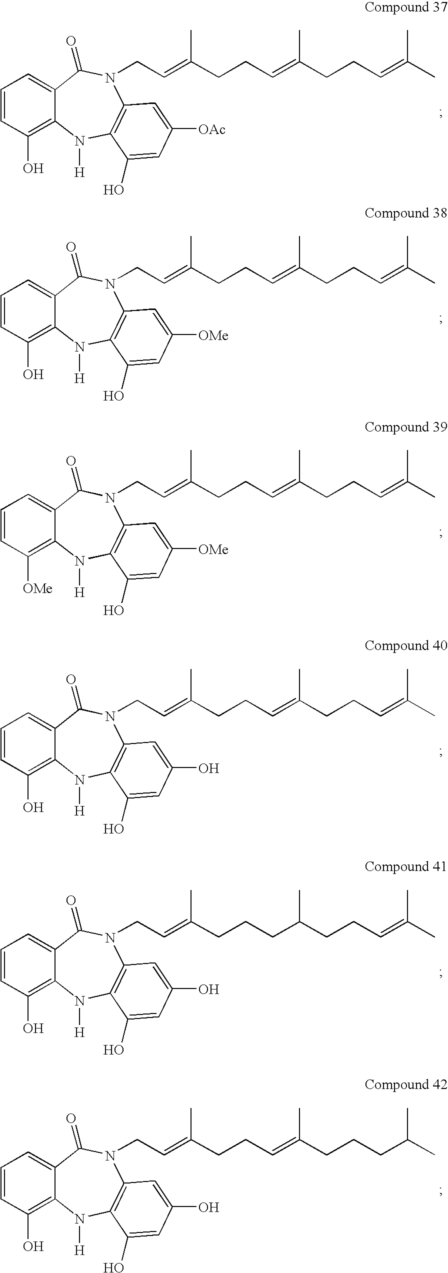

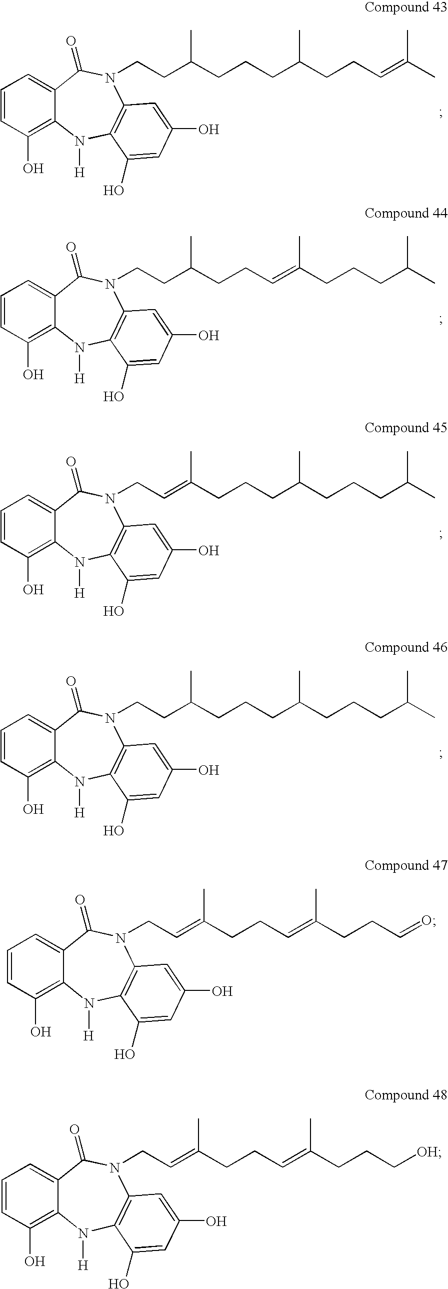

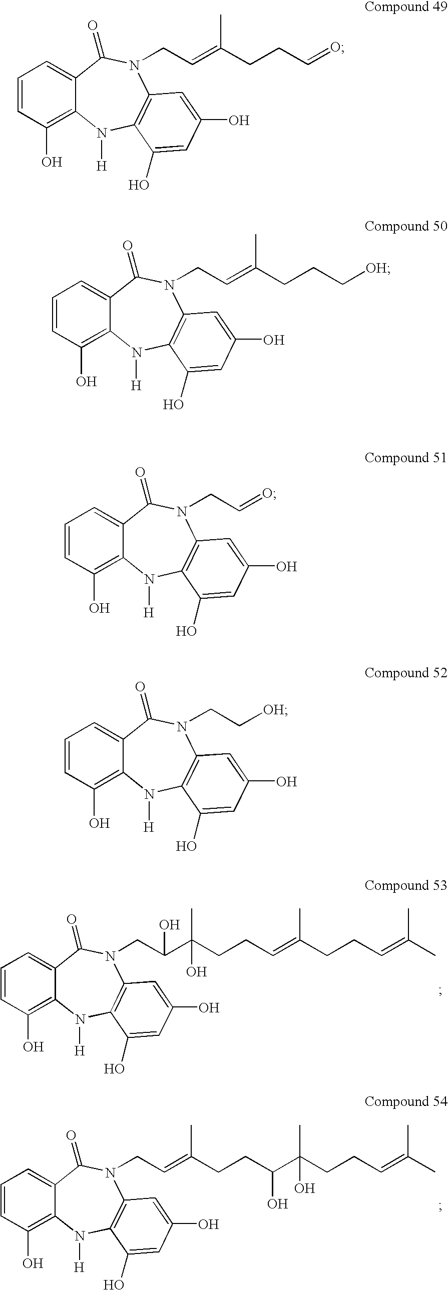

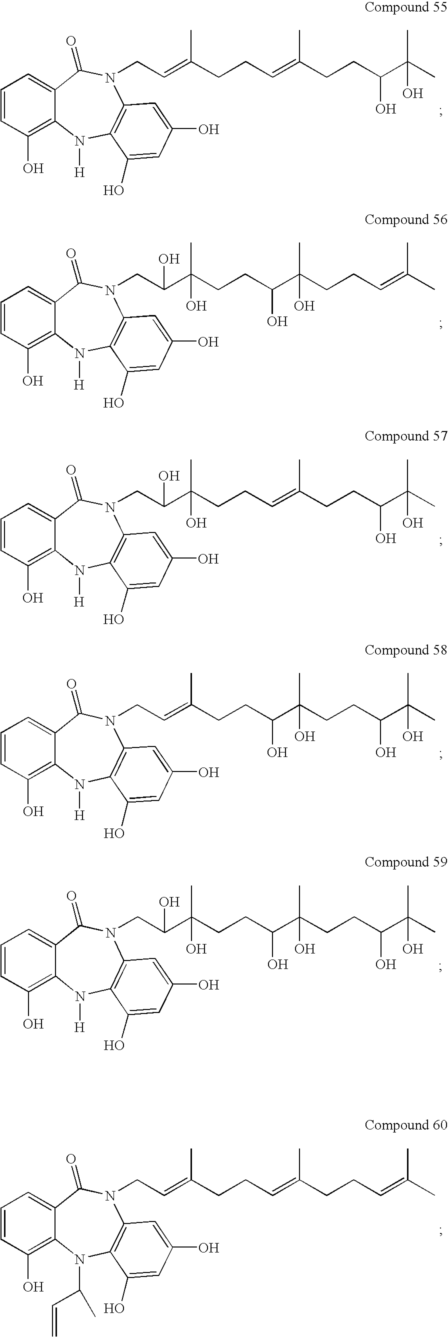

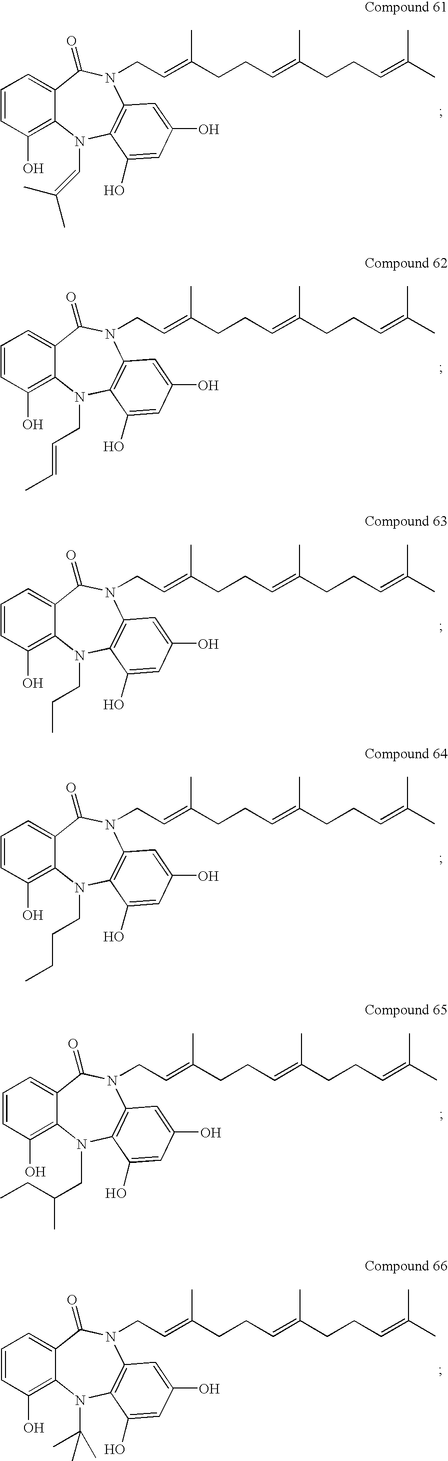

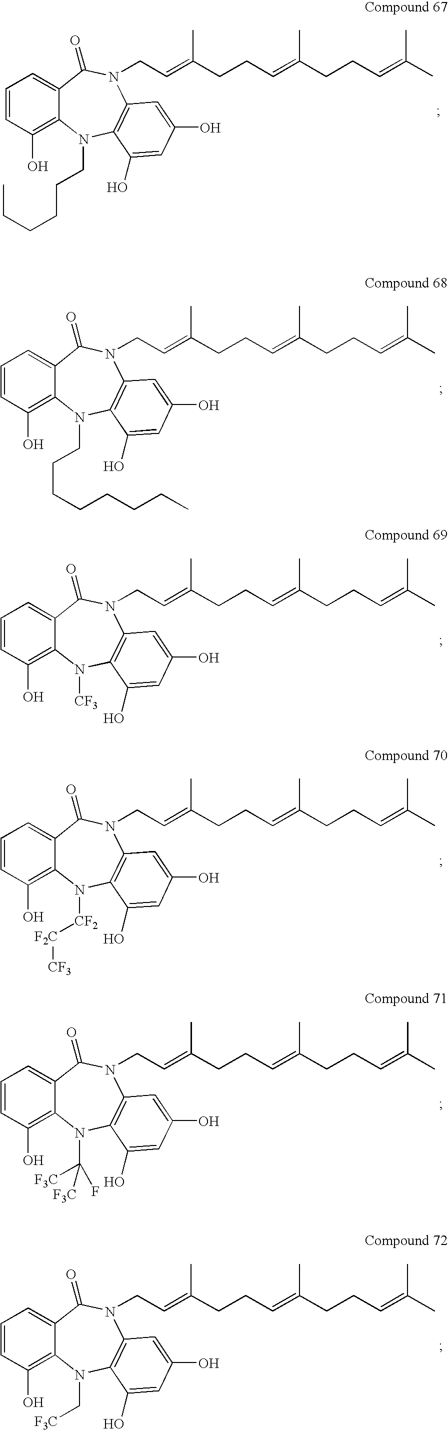

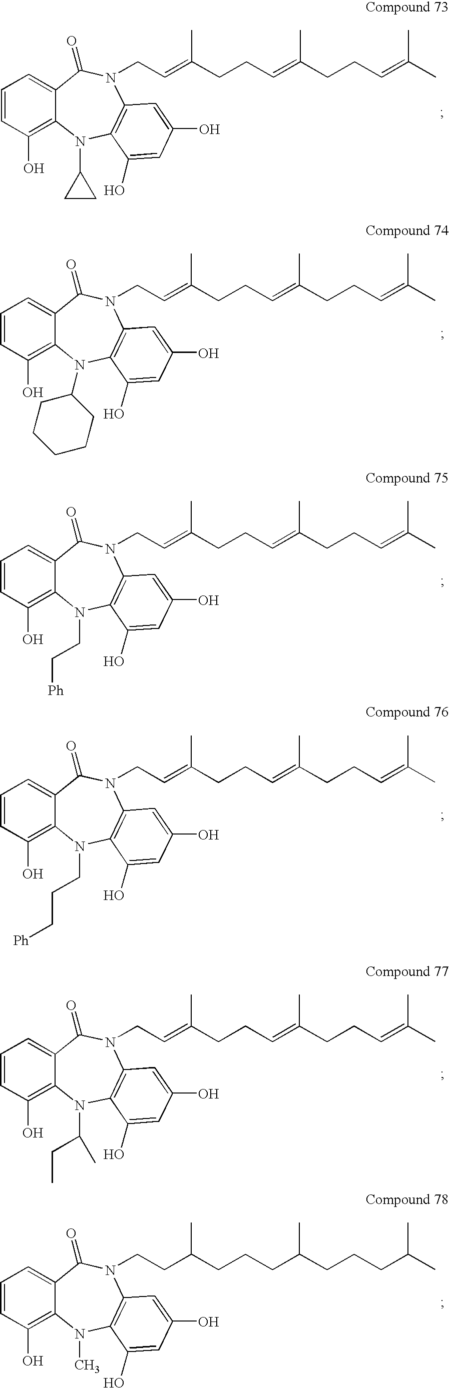

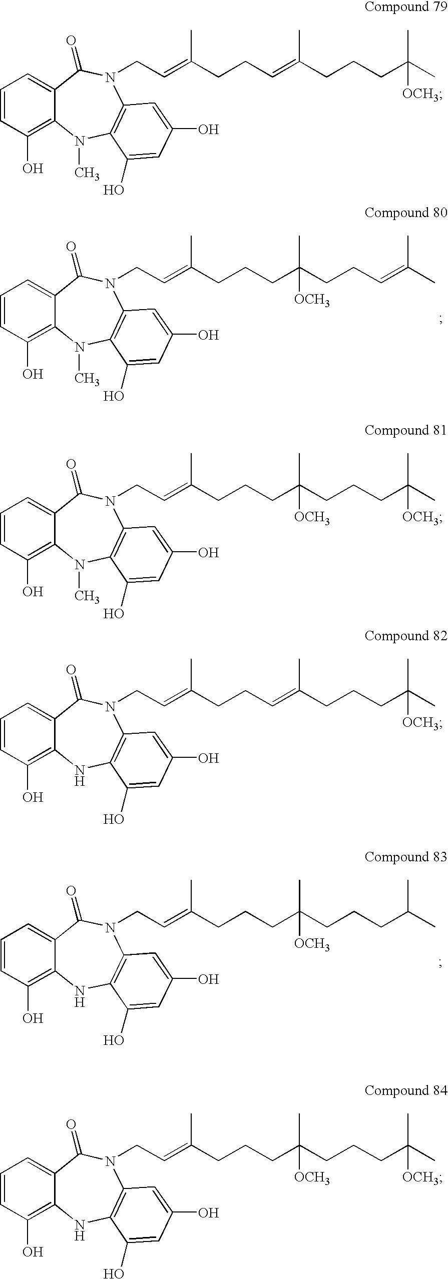

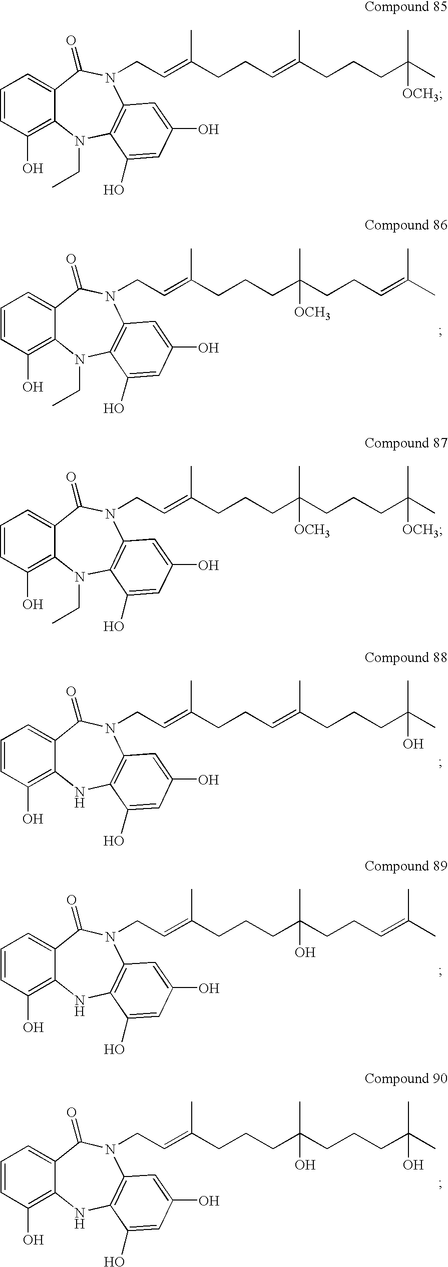

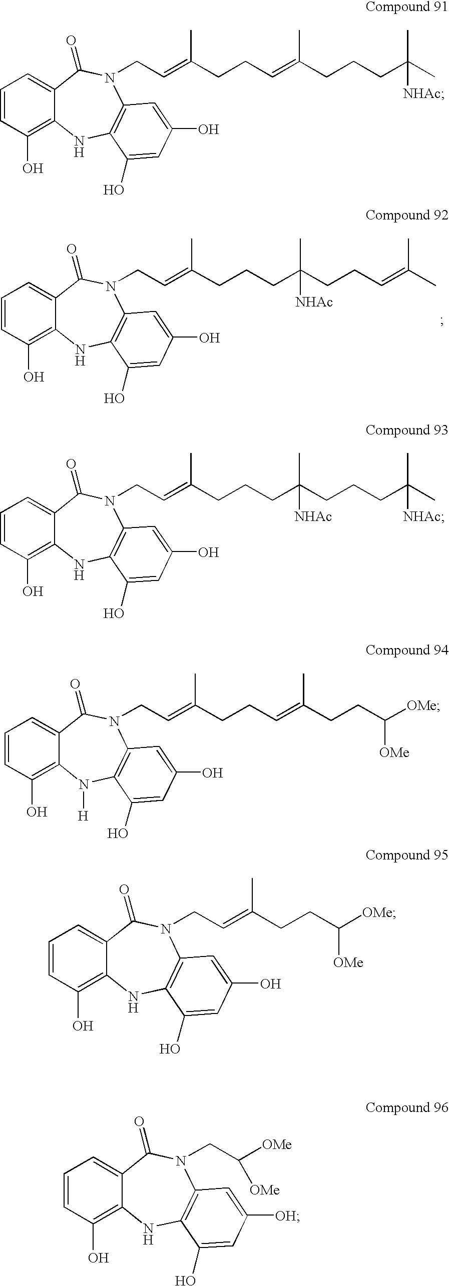

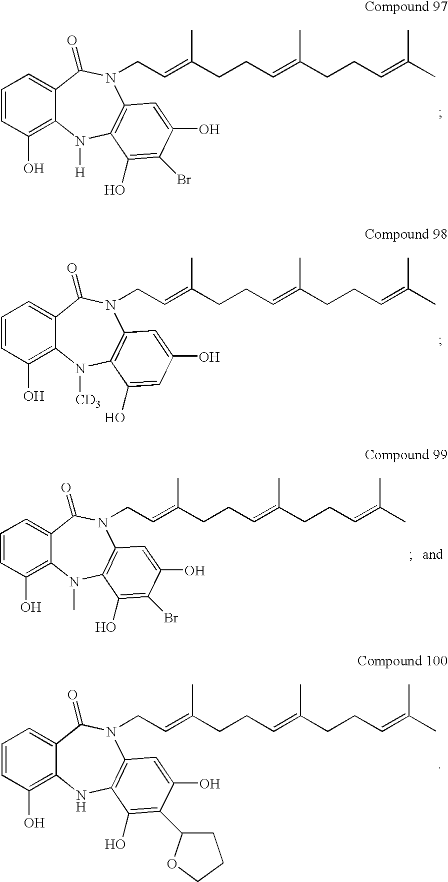

The compounds of Formula I are as defined below:

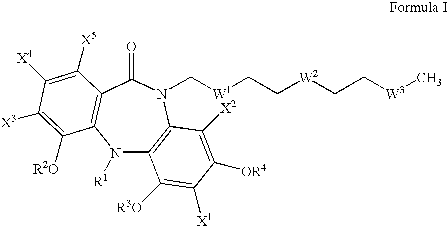

wherein,

W¹, W² and W³ are each independently selected from

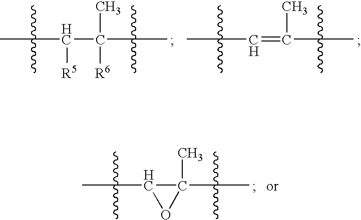

the chain from the tricycle terminates at W³, W² or W¹ with W³, W² or W¹ respectively being either —CH═O, —CH(OC₁₋₆alkyl)₂, —CH₂OH, —CH₂OC₁₋₆alkyl or C(O)OR⁷;

R¹ is selected from the group consisting of H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl, C₃₋₁₀heterocycloalkyl, C(O)H, C(O)C₁₋₁₀alkyl, C(O)C₂₋₁₀alkenyl, C(O)C₂₋₁₀alkynyl, C(O)C₆₋₁₀aryl, C(O)C₅₋₁₀heteroaryl, C(O)C₃₋₁₀cycloalkyl; C(O)C₃₋₁₀heterocycloalkyl and a C-coupled amino acid;

R², R³, and R⁴ are each independently selected from the group consisting of H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl, C₃₋₁₀heterocycloalkyl, C(O)H, C(O)C₁₋₁₀alkyl, C(O)C₂₋₁₀alkenyl, C(O)C₂₋₁₀alkynyl, C(O)C₆₋₁₀aryl, C(O)C₅₋₁₀heteroaryl, C(O)C₃₋₁₀cycloalkyl; C(O)C₃₋₁₀heterocycloalkyl and a C-coupled amino acid;

R⁵ and R⁶ are each independently selected from the group consisting of H, OH, OC₁₋₆alkyl, NH₂, NHC₁₋₆alkyl, N(C₁₋₆alkyl)₂, and NHC(O)C₁₋₆alkyl;

R⁷ is selected from the group consisting of H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl and C₃₋₁₀heterocycloalkyl;

X¹, X², X³, X⁴ and X⁵ are each H; or one of X¹, X², X³, X⁴ or X⁵ is halogen and the remaining ones are H; and

wherein, when any of R¹, R², R³, R⁴, R⁵, R⁶ and R⁷ comprises an alkyl, alkenyl, alkynyl, aryl, heteroaryl, cycloalkyl, or heterocycloalkyl group, then the alkyl, alkenyl, alkynyl, aryl, heteroaryl, cycloalkyl, or heterocycloalkyl group is optionally substituted with substituents selected from the group consisting of acyl, amino, acylamino, acyloxy, carboalkoxy, carboxy, carboxyamido, cyano, halo, hydroxyl, nitro, thio, C₁₋₆alkyl, C₂₋₇alkenyl, C₂₋₇alkynyl, C₃₋₁₀cycloalkyl, C₃₋₁₀heterocycloalkyl, C₆₋₁₀aryl, C₅₋₁₀heteroaryl, alkoxy, aryloxy, sulfinyl, sulfonyl, oxo, guanidino and formyl;

and an ester, ether, N-alkylated or N-acylated derivative, or a pharmaceutically acceptable salt, solvate or prodrug thereof.

The compounds of Formula II are as defined below:

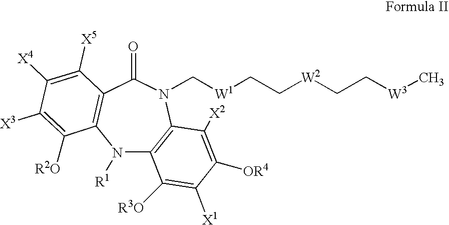

wherein,

W¹, W² and W³ are each independently selected from

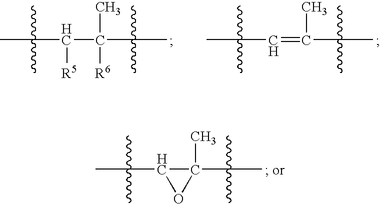

the chain from the tricycle terminates at W³, W² or W¹ with W³, W² or W¹ respectively being either —CH═O, —CH(OC₁₋₆alkyl)₂, —CH₂OH, —CH₂OC₁₋₆alkyl or C(O)OR⁷;

R¹ is selected from the group consisting of H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl, C₃₋₁₀heterocycloalkyl, C(O)H, C(O)C₁₋₁₀alkyl, C(O)C₂₋₁₀alkenyl, C(O)C₂₋₁₀alkynyl, C(O)C₆₋₁₀aryl, C(O)C₅₋₁₀heteroaryl, C(O)C₃₋₁₀cycloalkyl; C(O)C₃₋₁₀heterocycloalkyl and a C-coupled amino acid;

R², R³, and R⁴ are each independently selected from the group consisting of H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl, C₃₋₁₀heterocycloalkyl, C(O)H, C(O)C₁₋₁₀alkyl, C(O)C₂₋₁₀alkenyl, C(O)C₂₋₁₀alkynyl, C(O)C₆₋₁₀aryl, C(O)C₅₋₁₀heteroaryl, C(O)C₃₋₁₀cycloalkyl; C(O)C₃₋₁₀heterocycloalkyl and a C-coupled amino acid;

R⁵ and R⁶ are each independently selected from the group consisting of H, OH, OC₁₋₆alkyl, NH₂, NHC₁₋₆alkyl, N(C₁₋₆alkyl)₂, and NHC(O)C₁₋₆alkyl;

R⁷ is selected from the group consisting of H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl and C₃₋₁₀heterocycloalkyl;

X¹, X², X³, X⁴ and X⁵ are each H; or one of X¹, X², X³, X⁴ or X⁵ is halogen and the remaining ones are H; and

wherein, when any of R¹, R², R³, R⁴, R⁵, R⁶ and R⁷ comprises an alkyl, alkenyl, alkynyl, aryl, heteroaryl, cycloalkyl, or heterocycloalkyl group, then the alkyl, alkenyl, alkynyl, aryl, heteroaryl, cycloalkyl, or heterocycloalkyl group is optionally substituted with substituents selected from the group consisting of acyl, amino, acylamino, acyloxy, carboalkoxy, carboxy, carboxyamido, cyano, halo, hydroxyl, nitro, thio, C₁₋₆alkyl, C₂₋₇alkenyl, C₂₋₇alkynyl, C₃₋₁₀cycloalkyl, C₃₋₁₀heterocycloalkyl, C₆₋₁₀aryl, C₅₋₁₀heteroaryl, alkoxy, aryloxy, sulfinyl, sulfonyl, oxo, guanidino and formyl;

with the proviso that when W¹, W² and W³ are all —CH═C(CH₃)—, and R², R³ and R⁴ are all H, then R¹ is not H;

and an ester, ether, N-alkylated or N-acylated derivative, or a pharmaceutically acceptable salt, solvate or prodrug thereof.

The invention further encompasses a dibenzodiazepinone analogue obtained by a method comprising: a) cultivating a Micromonospora sp. strain selected from strains [S01]046 and 046-ECO11, wherein the cultivation is performed under aerobic conditions in a nutrient medium comprising at least one source of carbon atoms and at least one source of nitrogen atoms; and b) isolating a dibenzodiazepinone analogue from the bacteria cultivated in step (a).

The invention further encompasses a dibenzodiazepinone analogue derivative obtained by a method comprising: a) cultivating Micromonospora sp. strain selected from strains [S01]046 and 046-ECO11, wherein the cultivation is performed under aerobic conditions in a nutrient medium comprising at least one source of carbon atoms and at least one source of nitrogen atoms; b) isolating a dibenzodiazepinone analogue from the bacteria cultivated in step a), and c) chemically modifying the compound isolated in b) to produce a dibenzodiazepinone analogue derivative. In one embodiment the dibenzodiazepinone analogue is a compound of Formula I. In another embodiment, the dibenzodiazepinone analogue is a compound of Formula II. In a further embodiment, the dibenzodiazepinone analogue is one of the individual compounds of Compounds 1-100, the structures of which are set forth above.

The invention further encompasses a process for making a dibenzodiazepinone analogue, comprising cultivation of a Micromonospora sp. strain selected from strains 046-ECO11 and [S01]046, in a nutrient medium comprising at least one source of carbon atoms and at least one source of nitrogen atoms, isolation and purification of the analogue. In a subclass of this embodiment, the process further comprises the step of chemically modifying the isolated analogue to produce a dibenzodiazepinone analogue derivative.

In one embodiment, the cultivation occurs under aerobic conditions.

In another embodiment, the carbon atom and nitrogen atom sources are chosen from the components shown in Table 1.

In another embodiment, the cultivation is carried out at a temperature ranging from 18° C. to 40° C. In a further embodiment, the temperature range is 18° C. to 29° C.

In another embodiment, the cultivation is carried out at a pH ranging from 6 to 9.

The invention further encompasses a method for making a dibenzodiazepinone analogue derivative, comprising chemically modifying the dibenzodiazepinone analogue Compound 1, and optionally isolating and purifying the dibenzodiazepinone analogue derivative produced. In one embodiment, the chemical modification step comprises at least one step selected from N-alkylations, N-acylations, O-alkylations, O-acylations, and modifications of the double bonds of the farnesyl side chain including, hydrogenation, electrophilic additions (e.g., epoxidation, dihydroxylation, hydration, hydroalkoxylation, hydroamidation, and the like), and double bond cleavage, like ozonolysis, and reduction of the ozonolysis product. In a subclass of this embodiment, the farnesyl side chain modification reaction is partial (one or two double bonds modified) or complete (all three double bonds are modified).

The invention further encompasses a method of treating a diseases selected from pre-cancerous or cancerous conditions, inflammation, autoimmune diseases, infections, neurodegenerative diseases and stress, the method comprising administering a compound of Formula I to a mammal in need of such treatment. In one embodiment, the compound is a compound selected from Compounds 1 to 100. In another embodiment, the invention provides a method for treating a diseases selected from pre-cancerous or cancerous conditions, inflammation, autoimmune diseases, infections, neurodegenerative diseases and stress, the method comprising administering a compound of Formula I to a mammal in need of such treatment, with the proviso that the compound of Formula I is not Compound 1, or alternatively is not Compound 2, or alternatively is neither Compound 1 nor 2.

The invention further encompasses a method of inhibiting the growth of a cancer cell, the method comprising contacting the cancer cell with a compound of Formula I, such that growth of the cancer cell is inhibited. In one embodiment, the compound is a compound selected from Compounds 1 to 100. In another embodiment, the invention provides a method of inhibiting the growth of a cancer cell, the method comprising contacting the cancer cell with a compound of Formula I such that growth of the cancer cell is inhibited, with the proviso that the compound of Formula I is not Compound 1, or alternatively is not Compound 2, or alternatively is neither Compound 1 nor 2.

The invention further encompasses a method of inhibiting the growth of a cancer cell in a mammal, the method comprising administering a compound of Formula I to a mammal comprising a cancer cell, such that growth of the cancer cell is inhibited in the mammal. In one embodiment, the compound is a compound selected from Compounds 1 to 100. In another embodiment, the invention provides a method of inhibiting the growth of a cancer cell in a mammal, the method comprising administering a compound of Formula I to the mammal such that growth of the cancer cell is inhibited, with the proviso that the compound of Formula I is not Compound 1, or alternatively is not Compound 2, or alternatively is neither Compound 1 nor 2.

The invention further encompasses a method of treating a pre-cancerous or cancerous condition in a mammal, comprising the step of administering to the mammal a therapeutically effective amount of a compound of Formula I, such that a pre-cancerous or cancerous condition is treated. In one embodiment, the compound is a compound selected from Compounds 1 to 100.

In another embodiment, the invention provides a method of treating a pre-cancerous or cancerous condition in a mammal, the method comprising the step of administering to the mammal a therapeutically effective amount of a compound of Formula I such that a pre-cancerous or cancerous condition is treated, with the proviso that the compound of Formula I is not Compound 1, or alternatively is not Compound 2, or alternatively is neither Compound 1 nor 2.

The invention further encompasses the use of a compound of Formula I, or a pharmaceutically acceptable salt, solvate or prodrug thereof as an antitumor agent for the treatment of a pre-cancerous or cancerous condition in a mammal. In one embodiment, the compound is a compound selected from Compounds 1 to 100. In another embodiment, the invention provides use of a compound of Formula I, or a pharmaceutically acceptable salt or prodrug thereof as an antitumor agent for the treatment of a pre-cancerous or cancerous condition in a mammal, with the proviso that the compound of Formula I is not Compound 1, or alternatively is not Compound 2, or alternatively is neither Compound 1 nor 2.

The invention further encompasses the use of a compound of Formula I, or a pharmaceutically acceptable salt or prodrug thereof as an antineoplastic agent for the treatment of a proliferative disorder in a mammal. In one embodiment, the compound is a compound selected from Compounds 1 to 100. In another embodiment, the invention provides use of a compound of Formula I, or a pharmaceutically acceptable salt or prodrug thereof as an antineoplastic agent for the treatment of a proliferative disorder in a mammal, with the proviso that the compound of Formula I is not Compound 1, or alternatively is not Compound 2, or alternatively is neither Compound 1 nor 2.

The invention further encompasses the use of a compound of Formula I, or a pharmaceutically acceptable salt or prodrug thereof in the preparation of a medicament for the treatment of a pre-cancerous or cancerous condition in a mammal. In one embodiment, the compound is a compound selected from Compounds 1 to 100. In another embodiment, the invention provides use of a compound of Formula I, or a pharmaceutically acceptable salt or prodrug thereof in the preparation of a medicament for the treatment of a pre-cancerous or cancerous condition in a mammal, with the proviso that the compound of Formula I is not Compound 1, or alternatively is not Compound 2, or alternatively is neither Compound 1 nor 2.

The invention further encompasses a commercial package comprising a compound of Formula I, or a pharmaceutically acceptable salt or prodrug thereof, together with instructions for use in the treatment of a neoplasm or a pre-cancerous or cancerous condition. In one embodiment, the compound is a compound selected from Compounds 1 to 100. In one embodiment, the invention provides a commercial package comprising a compound of Formula I, or a pharmaceutically acceptable salt or prodrug thereof, together with instructions for use in the treatment of a neoplasm or a pre-cancerous or cancerous condition, with the proviso that the compound of Formula I is not Compound 1, or alternatively is not compound 2, or alternatively is neither Compound 1 nor 2.

The invention further encompasses the use of a compound of Formula I, or a pharmaceutically acceptable salt or prodrug thereof as a peripheral benzodiazepine receptor (PBR) binding agent for the treatment of a condition involving the PBR. The peripheral benzodiazepine receptor (PBR) is a well-characterized receptor known to be directly involved in diseases states. The PBR is involved in the regulation of immune responses. These diseases states include tumors, inflammatory diseases (such as rheumatoid arthritis and lupus), parasitic infections and neurodegenerative diseases (such as Alzheimer's, Huntington's and Multiple Sclerosis). See, for example, Casellas et al (2003) Current Med. Chem. 10, pp 1563-1572; Miettinen et al (1995) Cancer Res 55, pp 2691; Junck et al (1989) J. Ann. Neurol 26, pp 752; Gavish et al (1993) Clin. Neuropharm 16, no 5, pp 401-417; Papadopoulos et al (2003) Ann. Pharm. Fr 61, pp 30-50; Vowinckel et al (1997) J. Neurosci. Res 50, p 345; Gelhert et al (1997) Neurochem. Int 31, pp 705; Lacor et al (1999) Brain Res 815, pp 70). In one embodiment, the compound for use in the treatment of a condition involving the PBR is a compound selected from Compounds 1 to 100. In another embodiment, the compound for use in the treatment of a condition involving the PBR is a compound of Formula I with the proviso that the compound of Formula I is not Compound 1, or alternatively is not Compound 2, or alternatively is neither Compound 1 nor 2.

The invention further encompasses a method of reducing inflammation in a mammal, comprising administering to a mammal having inflammation a therapeutically effective amount of a compound of Formula I, such that the inflammation is reduced. In one embodiment, the compound is a compound selected from Compounds 1 to 100. In another embodiment, the compound for use as an anti-inflammatory is a compound of Formula I with the proviso that the compound of Formula I is not Compound 1, or alternatively is not Compound 2, or alternatively is neither Compound 1 nor 2.

The invention further encompasses the use of a compound of Formula I, or a pharmaceutically acceptable salt or prodrug thereof as a 5-Lipooxygenase (5-LO) inhibitor for the treatment of a condition involving the 5-LO enzyme. 5-Lipoxygenase (5-LO) catalyzes the oxidative metabolism of arachidonic acid to 5-hydroxyeicosatetraenoic acid (5-HETE), the initial reaction leading to formation of leukotrienes. Eicosanoids derived from arachidonic acid by the action of lipoxygenases or cyclooxygenases have been found to be involved in acute and chronic inflammatory diseases (i.e. asthma, multiple sclerosis, rheumatoid arthritis, ischemia, edema) as well in neurodegeneration (Alzheimer's disease), aging and various steps of carcinogenesis, including tumor promotion, progression and metastasis (Soberman et al (2003) J. Clin. Invest 111, no 8, pp 1107-1113). In one embodiment, the compound for use in the treatment of a condition involving the 5-LO enzyme is a compound selected from Compounds 1 to 100. In another embodiment, the compound for use as in the treatment of a condition involving the 5-LO enzyme is a compound of Formula I with the proviso that the compound of Formula I is not Compound 1, or alternatively is not Compound 2, or alternatively is neither Compound 1 nor 2.

Leukotriene, Cysteinyl (CysLT₁) receptor is involved in inflammation and CysLT₁-selective antagonists are used as treatment for bronchial asthma. CysLT₁ and 5-LO were known to be upregulated in colon cancer. See, for example, Lynch et al (1999) Nature 399(67387), pp 789; Nielsen et al (2003) Adv. Exp. Med. Biol vol 525, pp 201-4. In one embodiment, the invention provides a compound of Formula I for treatment of a condition involving the Leukotriene, Cysteinyl (CysLT₁) receptor. In one embodiment, the compound is a compound selected from Compounds 1 to 100. In another embodiment, the compound for use in the treatment of a condition involving CysLT₁ receptor is a compound of Formula I with the proviso that the compound of Formula I is not Compound 1, or alternatively is not Compound 2, or alternatively is neither Compound 1 nor 2.

Cyclooxygenase-2 (COX-2) enzyme is known to be produced only in response to injury or infection. It produces prostaglandins involved in inflammation and the immune response. Elevated levels of COX-2 in the body have been linked to cancer. See, for example, Wikstrom et al (2003) Biochem. Biophys. Res. Commun 302, no 2, pp 330-335. In one embodiment, the invention provides a compound of Formula I for treatment of a condition involving the COX-2 enzyme. In one embodiment, the compound is a compound selected from Compounds 1 to 100. In another embodiment, the compound for use as in the treatment of a condition involving the COX-2 enzyme is a compound of Formula I with the proviso that the compound of Formula I is not Compound 1, or alternatively is not Compound 2, or alternatively is neither Compound 1 nor 2.

AcylCoA-Cholesterol Acyltransferase (ACAT) is known to convert cholesterol to cholesteryl esters and is involved in the development of artheriosclerosis. See, for example, Kusunoki et al (2001), Circulation, pp 2604-2609. In one embodiment, the invention provides a compound of Formula I for treatment of a condition involving AcylCoA-Cholesterol Acyltransferase. In one embodiment, the compound is a compound selected from Compounds 1 to 100. In another embodiment, the compound for use as in the treatment of a condition involving AcylCoA-Cholesterol Acyltransferase is a compound of Formula I with the proviso that the compound of Formula I is not Compound 1, or alternatively is not Compound 2, or alternatively is neither Compound 1 nor 2.

The present invention further includes a method for evaluating the chemotherapeutic potential of a candidate molecule, comprising: (a) determining the ability of a candidate molecule to inhibit the in vitro growth of a cancer cell; (b) determining the ability of the candidate molecule to bind a cellular receptor produced by a cancer cell, wherein said receptor is produced in a greater amount by the cancer cell than by a normal cell; and (c) determining the ability of the candidate molecule to inhibit the activity of at least one protein member of the MAPK pathway, thereby evaluating a chemotherapeutic potential of a candidate molecule.

In one embodiment of this method, the candidate molecule is a non-conjugated compound, such as a small molecule, and includes a dibenzodiazepinone analogue or a dibenzodiazepinone analogue derivative of the present invention.

In a further embodiment of this method, the determining of (b) is determined by a competitive binding assay between the candidate molecule and a cognate ligand of said cellular receptor.

In an additional embodiment of this method, the receptor is a peripheral benzodiazepinone receptor (PBR).

In another embodiment of this method, the at least one protein member is encoded for by a proto-oncogene, preferably a protein having a GTPase activating activity. Proteins having a GTPase activating activity included members of the Ras GTPase family, such as Ras or Raf, preferably k-Ras.

The present invention is also directed to a compound identified as having each of activities (a), (b) and (c), where activities (a), (b) and (c) are: (a) the ability to inhibit the in vitro growth of a cancer cell; (b) the ability to bind a cellular receptor produced by a cancer cell, wherein said receptor is produced in a greater amount by the cancer cell than by a normal cell; and (c) the ability to inhibit the activity of at least one protein member of the MAPK pathway.

The present invention is further directed to a method for lessening a non-specific cytotoxicity side-effect of an anti-cancer treatment administered to a mammal, comprising administrating to a mammal having cancer and being treated with an anti-cancer treatment a molecule having each of activities (a), (b) and (c), wherein activities (a), (b) and (c) are: (a) the ability to inhibit the in vitro growth of a cancer cell; (b) the ability to bind a cellular receptor produced by a cancer cell, wherein said receptor is produced in a greater amount by the cancer cell than by a normal cell; and (c) the ability to inhibit the activity of at least one protein member of the MAPK pathway.

Similarly, the present invention is directed to a method for lessening a non-specific cytotoxicity side-effect of an anti-cancer treatment administered to a mammal, comprising administrating to a mammal having cancer and being treated with an anti-cancer treatment a molecule having each of activities (a), (b) and (c), wherein said molecule is not Compound 1, and wherein activities (a), (b) and (c) are: (a) the ability to inhibit the in vitro growth of a cancer cell; (b) the ability to bind a cellular receptor produced by a cancer cell, wherein said receptor is produced in a greater amount by the cancer cell than by a normal cell; and (c) the ability to inhibit the activity of at least one protein member of the MAPK pathway.

The present invention also includes a method of evaluating a candidate molecule, comprising: (a) determining the ability of a candidate molecule to bind to a peripheral benzodiazepinone receptor (PBR) with an IC₅₀ (μM) of less than 5; and (b) determining the ability of the candidate molecule to reduce a phosphorylation activity in a MAPK pathway in a cell upon application of said candidate molecule to said cell in a concentration range of 3 μM to 30 μM, thereby evaluating a candidate molecule.

In another embodiment of this method, the evaluation is an evaluation of anti-cancer activity.

In an additional embodiment of this method, the phosphorylation activity is reduced by inhibiting the activity of a Ras GTPase protein family member. In preferred embodiments, the Ras GTPase is Ras or k-Ras.

In yet another embodiment of this method, the candidate molecule is a compound of Formula I. In a related embodiment, the candidate molecule is a compound of Formula I but not Compound 1. Preferred candidate molecules include Compound 97, Compound 99 and Compound 100.

The present invention is also directed to a compound having activities (a) and (b), where activities (a) and (b) are: (a) the ability to bind to a peripheral benzodiazepinone receptor (PBR) with an IC₅₀ (μM) of less than 5; and (b) the ability to reduce a phosphorylation activity in a MAPK pathway in a cell upon application of said candidate molecule to said cell in a concentration range of 3 μM to 30 μM.

The present invention is also directed to a method for lessening a non-specific cytotoxicity side-effect of an anti-cancer treatment administered to a mammal, comprising administrating to a mammal having cancer and being treated with an anti-cancer treatment a molecule having each of activities (a) and (b), wherein activities (a) and (b) are: (a) the ability to bind to a peripheral benzodiazepinone receptor (PBR) with an IC₅₀ (μM) of less than 5; and (b) the ability to reduce a phosphorylation activity in a MAPK pathway in a cell upon application of said candidate molecule to said cell in a concentration range of 3 μM to 30 μM.

The present invention is further directed to a method for lessening a non-specific cytotoxicity side-effect of an anti-cancer treatment administered to a mammal, comprising administrating to a mammal having cancer and being treated with an anti-cancer treatment a molecule having each of activities (a) and (b) in accordance with the method of claim 12, wherein said molecule is not Compound 1, and wherein activities (a) and (b) are: (a) the ability to bind to a peripheral benzodiazepinone receptor (PBR) with an IC₅₀ (μM) of less than 5; and (b) the ability to reduce a phosphorylation activity in a MAPK pathway in a cell upon application of said candidate molecule to said cell in a concentration range of 3 μM to 30 μM.

The present invention is also directed to a compound of the formula:

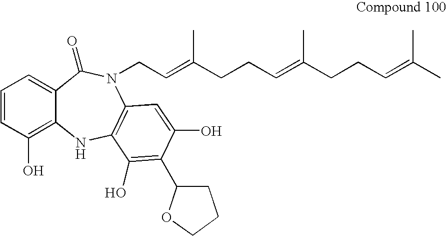

or a pharmaceutically acceptable salt thereof.

The present invention is also directed to a pharmaceutical composition comprising a compound of the formula:

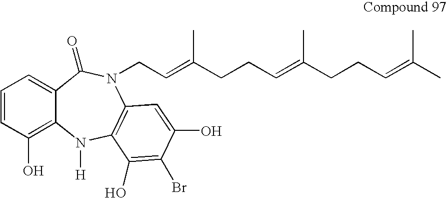

or a pharmaceutically acceptable salt thereof, together with a pharmaceutically acceptable carrier.

The present invention is also directed to a pharmaceutical composition comprising a compound of the formula:

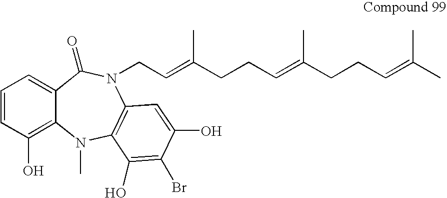

or a pharmaceutically acceptable salt thereof, together with a pharmaceutically acceptable carrier.

The present invention is additionally directed to a pharmaceutical composition comprising a compound of the formula:

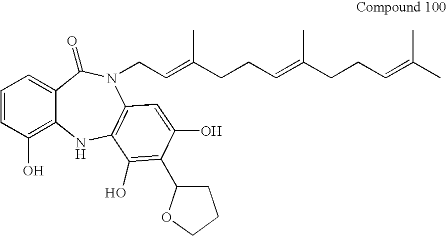

or a pharmaceutically acceptable salt thereof, together with a pharmaceutically acceptable carrier.

### BRIEF DESCRIPTION OF THE FIGURES

FIG. 1A-D: show the ¹H-NMR spectrum of Compound 100.

FIG. 2: shows the in vitro anti-inflammatory activity of Compound 1. Graph shows percent inhibition of 5-lipoxygenase activity plotted against the Log μM concentration of Compound 1 and NDGA. Graph shows the EC₅₀ of Compound 1 to be 0.93 μM.

FIG. 3: shows the pharmokinetic profiles of Compounds 1 and 2 in CD-1 mice following 30 mg/kg intravenous (IV) and intraperitoneal (IP) administrations.

FIG. 4A-B: shows in A. schatchard plot analysis of rat heart mitochondrial membrane using [³H]PK11195 as the specific ligand, and in B. binding displacement of [³H]PK11195 with Compound 1 (ECO-4601).

FIG. 5A-B: shows in A. In vivo PET images from rat brains before (upper image) and after (lower image) administration of ECO-4601 (“E-4601”), and in B. quantification of PET scan images during the 60 min PET analysis from a competitive binding study of ¹¹C-(R)-11195 before and after administration of ECO-4601 utilizing data from n=6 rats.

FIG. 6A-B: shows in A and B. In vivo PET images from rat brains before (A) and after (B) administration of ECO-4601, and in C. Bar graph plot results from a competitive binding study utilizing data from n=6 rats and showing a binding potential of ¹¹C-(R)-11195 before and after administration of ECO-4601.

FIG. 7: shows a bar graph plot of ECO-4601 concentrations in plasma and selected tissues obtained from n=6 rats treated CIV with ECO-4601 for 60 min.

FIG. 8: shows Western blot analysis of human breast MCF7 tumor cells extracts exposed to varying concentrations of ECO-4601 vs lovostatin for 18 h probed with Ras, RAPLA and HDJ2 specific antibodies (GAPDH was used as a loading control). Data indicates that ECO-4601 does not inhibit protein prenylation.

FIG. 9: shows Pull-down and Western blot analyses of human breast MCF7 tumor cells extracts exposed to varying concentrations of ECO-4601 for 18 h. Ras was immunodetected in the pull-down fraction and total fraction using a pan-Ras antibody.

FIG. 10A-B: shows in A. Western blot analysis of human breast MCF₇ and MDA-MB-231 tumor cells extracts exposed to 10 uM of ECO-4601 for different times as indicated and probed with p-Raf-1, Raf-1, p-ERK 1/2 and ERK 1/2 specific antibodies (GAPDH was used as a loading control), and in B. Western blot analysis of human glioma U87 MG and human prostate PC3 tumor cells extracts exposed to 10 uM of ECO-4601 for different times as indicated and probed with p-Raf-1, Raf-1, p-ERK 1/2 and ERK 1/2 specific antibodies (GAPDH was used as a loading control).

FIG. 11A-B. shows in A. Western blot analysis of human pancreatic MiaPaCa-2 tumor cells extracts exposed to varying concentrations of ECO-4601 for 18 h as indicated and probed with Raf-1, p-MEK, MEK and PARP specific antibodies (GAPDH was used as a loading control), and in B. a densitometric quantification of the Western blots.

FIG. 12: Shows tumor growth curves of mice bearing human pancreatic MiaPaCa-2 tumors treated with vehicle (negative control), gemcitabine (positive control) or ECO-4601.

FIG. 13: shows Raf-1 protein tumor levels after 5 days of treatment with either vehicle control of ECO-4601. The tumors were obtained from 5 extra animals per group at day 23 of the experiment described in FIG. 12.

FIG. 14: shows the results of an ERK phosphorylation ELISA assay, where “4601” is Compound 1, “4625” is Compound 97, “4657” is Compound 99 and “4687” is Compound 100.

FIG. 15: shows a model proposed for the inhibition of the Ras-MAPK pathway by ECO-4601. Following ligand binding, the EGFR (as well as other receptors from this family) undergoes dimerization and autophosphorylation on several tyrosine residues in the cytoplasmic tails. This provides docking sites for SH2 domains of adaptor proteins such as Shc and GRB2 (growth-factor receptor-bound-protein 2). In cooperation with GRB2, the guanine-nucleotide exchange factor SOS activates Ras by catalyzing the replacement of GDP with GTP. Activated Ras relays its signal downstream through the activation of c-Raf, MEK1/2 and ERK1/2. Once activated, ERK1/2 translocate to the nucleus where they phosphorylate and activate a variety of substrates that control cell growth and differentiation. ECO-4601 inhibits this pathway by acting post Ras protein prenylation and prior to Raf phosphorylation.

### DETAILED DESCRIPTION OF THE INVENTION

The present invention relates to novel dibenzodiazepinone analogues, including the compounds of Formula I, the compounds of Formula II, and each of Compounds 1-100. The present invention also includes derivatives of each of these compounds.

An exemplary compound of the present invention is the dibenzodiazepinone analogue of Compound 1. Compound 1 is isolated from strains of actinomycetes, Micromonospora sp. 046-ECO11 and [S01]046. These organisms were deposited on Mar. 7, 2003, and Dec. 23, 2003, respectively, with the International Depositary Authority of Canada (IDAC), Bureau of Microbiology, Health Canada, 1015 Arlington Street, Winnipeg, Manitoba, Canada R3E,3R2, under Accession Nos. IDAC 070303-01 and IDAC 231203-01, respectively.

The invention further relates to pharmaceutically acceptable salts, solvates and prodrugs of the dibenzodiazepinone analogues and derivatives of the present invention, and to methods for obtaining such compounds.

One method of obtaining the dibenzodiazepinone analogues of the present invention is by cultivating Micromonospora sp. strain 046-ECO11 or [S01]046, or a mutant or a variant thereof, under suitable Micromonospora culture conditions, preferably using the fermentation protocol described hereinbelow, to thereby obtain the dibenzodiazepinone analogues. Chemical modification may then be used to produce the derivatives of the dibenzodiazepinone analogues obtained by isolation from the fermentation procedure.

The invention also relates to a method for producing novel dibenzodiazepinone analogue derivatives of the compounds of Formula II, by chemical modification of the dibenzodiazepinone analogue obtained from fermentation and isolation.

The present invention also relates to pharmaceutical compositions comprising a compound of Formula I and its pharmaceutically acceptable salts, solvates and derivatives. Compounds of Formula I are useful as pharmaceuticals, in particular for use as an inhibitor of cancer cell growth, and mammalian lipoxygenase.

The following detailed description discloses how to make and use the compounds of Formula I and compositions containing these compounds to inhibit tumor growth and/or specific disease pathways.

Accordingly, certain aspects of the present invention relate to pharmaceutical compositions comprising the dibenzodiazepinone compounds of the present invention together with a pharmaceutically acceptable carrier, and methods of using the pharmaceutical compositions to treat diseases, including cancer, and chronic and acute inflammation, autoimmune diseases, and neurodegenerative diseases.

### I. DEFINITIONS

All technical and scientific terms used herein have the same meaning as commonly understood by one of ordinary skill in the art to which this invention belongs. For convenience, the meaning of certain terms and phrases used in the specification, examples, and appended claims, are provided below.

As used herein, the term “farnesyl dibenzodiazepinone” refers to Compound 1, namely 10-farnesyl-4,6,8-trihydroxy-5,10-dihydrodibenzo[b,e][1,4]diazepin-11-one, also referred to as ECO-4601.

As used herein, the terms “dibenzodiazepinone analogue(s)” and equivalent expressions refer to a class of dibenzodiazepinone molecules containing a farnesyl moiety or being derived from a farnesyl moiety, and pharmaceutically acceptable salts, solvates and prodrugs thereof. The term includes each of Compounds 1-100, the compounds of Formula I, and the compounds of Formula II as well as a pharmaceutically acceptable salt, solvate or prodrug of any of these compounds. As used herein, the term “dibenzodiazepinone analogues” includes compounds of this class that can be used as intermediates in chemical syntheses and variants containing different isotopes than the most abundant isotope of an atom (e.g., D replacing H, ¹³C replacing ¹²C, etc). The compounds of the invention are also sometimes referred as “active ingredients”.

As used herein, the “dibenzodiazepinone analogue derivatives”, “chemical derivatives” of dibenzodiazepinone analogues, “derivatives” of dibenzodiazepinone analogues, and equivalent expressions, refer to a class of dibenzodiazepinone molecules produced by chemical modification of the dibenzodiazepinone analogues of the present invention, and to pharmaceutically acceptable salts, solvates and prodrugs thereof. The term includes derivatives produced by chemical modification of each of Compounds 1-100, the compounds of Formula I, and the compounds of Formula II, as well as a pharmaceutically acceptable salt, solvate or prodrug of the derivatives.

As used herein, the term “chemical modification” refers to one or more steps of modifying a dibenzodiazepinone analogue, referred to as “starting material”, by chemical synthesis. Preferred analogues for use as starting materials in a chemical modification process are Compounds 1 to 100, more preferably Compounds 1, 2, 46, 97, 99 and 100. Examples of chemical modification steps include N-alkylations, N-acylations, O-alkylations, O-acylations, aromatic halogenation, and modifications of the double bonds of the farnesyl side chain including, hydrogenation, electrophilic additions (e.g., epoxidation, dihydroxylation, hydration, hydroalkoxylation, hydroamidation, and the like), and double bond cleavage like ozonolysis, and reduction of ozonolysis product. Farnesyl side chain modification reaction can be partial (one or two double bonds modified) or complete (three double bonds modified). Chemical modification steps are also defined in the Schemes of Section 111B, and exemplified in Examples 4 to 9 and Example 15.

The term “ether” refers to a dibenzodiazepinone analogue derivative obtained by the replacement of a hydrogen atom from an alcohol by an R′ replacement group by an O-alkylation reaction as defined in Scheme 1 (a) below. More particularly, the term ether encompasses ethers of the alcohols in positions 4, 6, and 8 (see Examples 3-9 for atom numbering).

The term “ester” refers to a dibenzodiazepinone analogue derivative obtained by the replacement of a hydrogen atom from an alcohol by a C(O)R″ replacement group by an O-acylation reaction as defined in Scheme 1 (b) below. The term ester also encompasses ester equivalents including, without limitation, carbonate, carbamate, and the like. More particularly, the term “ester” encompasses esters of the alcohols in positions 4, 6, and 8 (see Examples 3-9 for atom numbering).

The term “N-alkylated derivative” refers to a dibenzodiazepinone analogue derivative obtained by the replacement of a hydrogen atom of an amine by an R replacement group by an N-alkylation reaction as defined in Scheme 2(a) below. More particularly, the term “N-alkylated derivative” encompasses derivatives of the amine in position 5 (see Examples 3-9 for atom numbering).

The term “N-acylated derivative” refers to a dibenzodiazepinone analogue derivative obtained by the replacement of a hydrogen atom of an amine by a C(O)R replacement group by an N-acylation reaction as defined in Scheme 2(b) below. The term N-acylated derivative further encompasses amide equivalents such as, without limitation, urea, guanidine, and the like. More particularly, the term “N-acylated derivative” encompasses derivatives of the amine in position 5 (see Examples 3-9 for atom numbering).

The term “receptor” refers to a protein located on the surface or inside a cell that may interact with a different molecule, known as a ligand, to initiate or inhibit a biological response.

As used herein, the term “ligand” refers to a molecule or compound that has the capacity to bind to a receptor and modulate its activity.

As used herein, the terms “binder”, “receptor binder” or “binding agent” refers to a compound of the invention acting as a ligand. The binding agent can act as an agonist, or an antagonist of the receptor. An agonist is a drug which binds to a receptor and activates it, producing a pharmacological response (e.g. contraction, relaxation, secretion, enzyme activation, etc.). An antagonist is a drug which counteracts or blocks the effects of an agonist, or a natural ligand. Antagonism can be competitive and reversible (i.e. it binds reversibly to a region of the receptor in competition with the agonist.) or competitive and irreversible (i.e. antagonist binds covalently to the receptor, and no amount of agonist can overcome the inhibition). Other types of antagonism are non-competitive antagonism where the antagonist binds to an allosteric site on the receptor or an associated ion channel.

As used herein, the term “enzyme inhibitor” or “inhibitor” refers to a chemical that disables an enzyme and inhibits it from performing its normal function.

As used herein, abbreviations have their common meaning. Unless otherwise noted, the abbreviations “Ac”, “Me”, “Et”, “Pr”, “i-Pr”, “Bu”, “Bz” and “Ph”, respectively refer to acetyl, methyl, ethyl, propyl (n- or iso-propyl), iso-propyl, butyl (n-, iso-, sec- or tert-butyl), benzoyl and phenyl. Abbreviations in the specification correspond to units of measure, techniques, properties or compounds as follows: “RT” means retention time, “min” means minutes, “h” means hour(s), “μL” means microliter(s), “mL” means milliliter(s), “mM” means millimolar, “M” means molar, “mmole” means millimole(s), “eq” means molar equivalent(s). “High Pressure Liquid Chromatography” and “High Performance Liquid Chromatography” are abbreviated HPLC.

The term “alkyl” refers to linear, branched or cyclic, saturated hydrocarbon groups. Examples of alkyl groups include, without limitation, methyl, ethyl, n-propyl, isopropyl, n-butyl, pentyl, hexyl, heptyl, cyclopentyl, cyclohexyl, cyclohexylmethyl, and the like. Alkyl groups may optionally be substituted with substituents selected from acyl, amino, acylamino, acyloxy, carboalkoxy, carboxy, carboxyamido, cyano, halo, hydroxyl, nitro, thio, alkyl, alkenyl, alkynyl, cycloalkyl, heterocycloalkyl, aryl, heteroaryl, alkoxy, aryloxy, sulfinyl, sulfonyl, oxo, guanidino and formyl.

The term “C₁ nalkyl”, wherein n is an integer from 2 to 12, refers to an alkyl group having from 1 to the indicated “n” number of carbons. The C₁₋ₙalkyl can be cyclic or a straight or branched chain.

The term “alkenyl” refers to linear, branched or cyclic unsaturated hydrocarbon groups containing, from one to six carbon-carbon double bonds. Examples of alkenyl groups include, without limitation, vinyl, 1-propene-2-yl, 1-butene-4-yl, 2-butene-4-yl, 1-pentene-5-yl and the like. Alkenyl groups may optionally be substituted with substituents selected from acyl, amino, acylamino, acyloxy, carboalkoxy, carboxy, carboxyamido, cyano, halo, hydroxyl, nitro, thio, alkyl, alkenyl, alkynyl, cycloalkyl, heterocycloalkyl, aryl, heteroaryl, alkoxy, aryloxy, sulfinyl, sulfonyl, formyl, oxo and guanidino. The double bond portion(s) of the unsaturated hydrocarbon chain may be either in the cis or trans configuration.

The term “C₂₋ₙalkenyl”, wherein n is an integer from 3 to 12, refers to an alkenyl group having from 2 to the indicated “n” number of carbons. The C₂₋ₙalkenyl can be cyclic or a straight or branched chain.

The term “alkynyl” refers to linear, branched or cyclic unsaturated hydrocarbon groups containing at least one carbon-carbon triple bond. Examples of alkynyl groups include, without limitation, ethynyl, 1-propyne-3-yl, 1-butyne-4-yl, 2-butyne-4-yl, 1-pentyne-5-yl and the like. Alkynyl groups may optionally be substituted with substituents selected from acyl, amino, acylamino, acyloxy, carboalkoxy, carboxy, carboxyamido, cyano, halo, hydroxyl, nitro, thio, alkyl, alkenyl, alkynyl, cycloalkyl, heterocycloalkyl, aryl, heteroaryl, alkoxy, aryloxy, sulfinyl, sulfonyl, formyl, oxo and guanidine.

The term “C₂₋ₙalkynyl”, wherein n is an integer from 3 to 12, refers to an alkynyl group having from 2 to the indicated “n” number of carbons. The C₂₋ₙalkynyl can be cyclic or a straight or branched chain.

The term “cycloalkyl” or “cycloalkyl ring” refers to an alkyl group, as defined above, further comprising a saturated or partially unsaturated carbocyclic ring in a single or fused carbocyclic ring system having from three to fifteen ring members. Examples of cycloalkyl groups include, without limitation, cyclopropyl, cyclobutyl, cyclopentyl, cyclopenten-1-yl, cyclopenten-2-yl, cyclopenten-3-yl, cyclohexyl, cyclohexen-1-yl, cyclohexen-2-yl, cyclohexen-3-yl, cycloheptyl, bicyclo[4,3,0]nonanyl, norbornyl, and the like. Cycloalkyl groups may optionally be substituted with substituents selected from acyl, amino, acylamino, acyloxy, carboalkoxy, carboxy, carboxyamido, cyano, halo, hydroxyl, nitro, thio, alkyl, alkenyl, alkynyl, cycloalkyl, heterocycloalkyl, aryl, heteroaryl, alkoxy, aryloxy, sulfinyl, sulfonyl and formyl.

The term “C₃₋ₙcycloalkyl”, wherein n is an integer from 4 to 15, refers to a cycloalkyl ring or ring system or having from 3 to the indicated “n” number of carbons.

The term “heterocycloalkyl”, “heterocyclic” or “heterocycloalkyl ring” refers to a cycloalkyl group, as defined above, further comprising one to four hetero atoms (e.g. N, O, S, P) or hetero groups (e.g. NH, NR^{x}, PO₂, SO, SO₂) in a single or fused heterocyclic ring system having from three to fifteen ring members (e.g. tetrahydrofuranyl has five ring members, including one oxygen atom). Examples of a heterocycloalkyl, heterocyclic or heterocycloalkyl ring include, without limitation, pyrrolidino, tetrahydrofuranyl, tetrahydrodithienyl, tetrahydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3,1,0]hexanyl, 3-azabicyclo[4,1,0]heptanyl, 3H-indolyl, and quinolizinyl. The foregoing heterocycloalkyl groups, as derived from the compounds listed above, may be C-attached or N-attached where such is possible. Heterocycloalkyl, heterocyclic or heterocycloalkyl ring may optionally be substituted with substituents selected from acyl, amino, acylamino, acyloxy, oxo, thiocarbonyl, imino, carboalkoxy, carboxy, carboxyamido, cyano, halo, hydroxyl, nitro, thio, alkyl, alkenyl, alkynyl, cycloalkyl, heterocycloalkyl, aryl, heteroaryl, alkoxy, aryloxy, sulfinyl, sulfonyl and formyl.

The term “C₃₋ₙheterocycloalkyl”, wherein n is an integer from 4 to 15, refers to a heterocycloalkyl group having from 3 to the indicated “n” number of atoms in the cycle and at least one hetero group as defined above.

The terms “halo” or “halogen” refers to bromine, chlorine, fluorine or iodine substituents.

The term “aryl” or “aryl ring” refers to common aromatic groups having “4n+2” electrons, wherein n is an integer from 1 to 3, in a conjugated monocyclic or polycyclic system and having from five to fourteen ring atoms. Aryl may be directly attached, or connected via a C₁₋₃alkyl group (also referred to as aralkyl). Examples of aryl include, without limitation, phenyl, benzyl, phenethyl, 1-phenylethyl, tolyl, naphthyl, biphenyl, terphenyl, and the like. Aryl groups may optionally be substituted with one or more substituent group selected from acyl, amino, acylamino, acyloxy, azido, alkythio, carboalkoxy, carboxy, carboxyamido, cyano, halo, hydroxyl, nitro, thio, alkyl, alkenyl, alkynyl, cycloalkyl, heterocyclyl, aryl, heteroaryl, alkoxy, aryloxy, sulfinyl, sulfonyl and formyl.

The term “C₅₋ₙaryl”, wherein n is an integer from 5 to 14, refers to an aryl group having from 5 to the indicated “n” number of atoms, including carbon, nitrogen, oxygen and sulfur. The C₅₋ₙaryl can be mono or polycyclic.

The term “heteroaryl” or “heteroaryl ring” refers to an aryl ring, as defined above, further containing one to four heteroatoms selected from oxygen, nitrogen, sulphur or phosphorus. Examples of heteroaryl include, without limitation, pyridyl, imidazolyl, pyrimidinyl, pyrazolyl, triazolyl, tetrazolyl, furyl, thienyl, isooxazolyl, thiazolyl, oxazolyl, isothiazolyl, pyrrollyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl groups. Heteroaryl may optionally be substituted with one or more substituent group selected from acyl, amino, acylamino, acyloxy, azido, alkythio, carboalkoxy, carboxy, carboxyamido, cyano, halo, hydroxyl, nitro, thio, alkyl, alkenyl, alkynyl, cycloalkyl, heterocyclyl, aryl, heteroaryl, alkoxy, aryloxy, sulfinyl, sulfonyl and formyl. Heteroaryl may be directly attached, or connected via a C₁₋₃alkyl group (also referred to as heteroaralkyl). The foregoing heteroaryl groups, as derived from the compounds listed above, may be C-attached or N-attached where such is possible.

The term “C₅₋ₙheteroaryl”, wherein n is an integer from 5 to 14, refers to an heteroaryl group having from 5 to the indicated “n” number of atoms, including carbon, nitrogen, oxygen and sulphur atoms. The C₅₋ₙheteroaryl can be mono or polycyclic.

The term “amino acid” refers to an organic acid containing an amino group. The term includes both naturally occurring and synthetic amino acids; therefore, the amino group can be but is not required to be, attached to the carbon next to the acid. A C-coupled amino acid substituent is attached to the heteroatom (nitrogen or oxygen) of the parent molecule via its carboxylic acid function. C-coupled amino acid forms an ester with the parent molecule when the heteroatom is oxygen, and an amide when the heteroatom is nitrogen. Examples of amino acids include, without limitation, alanine, valine, leucine, isoleucine, proline, phenylalanine, tryptophan, methionine, glycine, serine, threonine, cysteine, asparagine, glutamine, tyrosine, histidine, lysine, arginine, aspartic acid, glutamic acid, desmosine, ornithine, 2-aminobutyric acid, cyclohexylalanine, dimethylglycine, phenylglycine, norvaline, norleucine, hydroxylysine, allo-hydroxylysine, hydroxyproline, isodesmosine, allo-isoleucine, ethylglycine, beta-alanine, aminoadipic acid, aminobutyric acid, ethyl asparagine, and N-methyl amino acids. Amino acids can be pure L or D isomers or mixtures of L and D isomers.

The compounds of the present invention can possess one or more asymmetric carbon atoms and can exist as optical isomers forming mixtures of racemic or non-racemic compounds. The compounds of the present invention are useful as single isomers or as a mixture of stereochemical isomeric forms. Diastereoisomers, i.e., nonsuperimposable stereochemical isomers, can be separated by conventional means such as chromatography, distillation, crystallization or sublimation. The optical isomers can be obtained by resolution of the racemic mixtures according to conventional processes, including chiral chromatography (e.g. HPLC), immunoassay techniques, or the use of covalently (e.g. Mosher's esters) or non-covalently (e.g. chiral salts) bound chiral reagents to respectively form a diastereomeric ester or salt, which can be further separated by conventional methods, such as chromatography, distillation, crystallization or sublimation. The chiral ester or salt is then cleaved or exchanged by conventional means, to recover the desired isomer(s).

The invention encompasses isolated or purified compounds. An “isolated” or “purified” compound refers to a compound which represents at least 10%, 20%, 50%, 80%, 81%, 82%, 83%, 84%, 85%, 86%, 87%, 88%, 89%, 90%, 91%, 92%, 93%, 94%, 95%, 96%, 97%, 98% or 99% of the mixture by weight, provided that the mixture comprising the compound of the invention has demonstrable (i.e. statistically significant) biological activity including cytostatic, cytotoxic, enzyme inhibitory or receptor binding action when tested in conventional biological assays known to a person skilled in the art.

The term “pharmaceutically acceptable salt” refers to nontoxic salts synthesized from a compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, methanol, ethanol, isopropanol, or acetonitrile are preferred. Another method for the preparation of salts is by the use of ion exchange resins. The term “pharmaceutically acceptable salt” includes both acid addition salts and base addition salts, either of the parent compound or of a prodrug or solvate thereof. The nature of the salt is not critical, provided that it is pharmaceutically acceptable. Exemplary acids used in acid addition salts include, without limitation, hydrochloric, hydrobromic, hydroiodic, nitric, carbonic, sulfuric, sulfonic, phosphoric, formic, acetic, citric, tartaric, succinic, oxalic, malic, glutamic, propionic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactaric, galacturonic acid and the like. Suitable pharmaceutically acceptable base addition salts include, without limitation, metallic salts made from aluminium, calcium, lithium, magnesium, potassium, sodium and zinc or organic salts, such as those made from N,N′-dibenzylethylenediamine, chloroprocaine, choline, diethanolamine, ethylenediamine, N-methylglucamine, lysine, procaine and the like. Additional examples of pharmaceutically acceptable salts are listed in Berge et al (1977) Journal of Pharmaceutical Sciences vol 66, no 1, pp 1-19.

The term “solvate” refers to a physical association of a compound of this invention with one or more solvent molecules, whether organic or inorganic. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Exemplary solvates include hydrates, ethanolates, methanolates, hemiethanolates, and the like.

The term “pharmaceutically acceptable prodrug” means any pharmaceutically acceptable ester, salt of an ester or any other derivative of a compound of this invention, which upon administration to a recipient, is capable of providing, either directly or indirectly, a compound of this invention or a biologically active metabolite or residue thereof. Particularly favored salts or prodrugs are those with improved properties, such as solubility, efficacy, or bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system) relative to the parent species. As used herein, a prodrug is a drug having one or more functional groups covalently bound to a carrier wherein metabolic or chemical release of the drug occurs in vivo when the drug is administered to a mammalian subject. Pharmaceutically acceptable prodrugs of the compounds of this invention include derivatives of hydroxyl groups such as, without limitation, acyloxymethyl, acyloxyethyl and acylthioethyl ethers, esters, amino acid esters, phosphate esters, sulfonate and sulfate esters, and metal salts, and the like.

### II. COMPOUNDS OF THE INVENTION

In one aspect, the invention relates to novel dibenzodiazepinone analogues and derivatives thereof, referred to herein as the compounds of the invention, and to pharmaceutically acceptable salts, solvates and prodrugs thereof.

The compounds of the invention may be characterized as any one of Compounds 1-100 and derivatives thereof produced by the chemical modifications as defined herein. Compounds 2 to 12, 14, 17, 18, 46, 63, 64, 67, 77, 78, 80, 82 to 85, 87, 89, 92, and 95 to 98 may be characterized by any one of their physicochemical and spectral properties, such as mass and NMR, detailed in Example 4 through Example 9.

In another aspect, the invention relates to dibenzodiazepinone analogues and derivatives thereof, represented by Formula I:

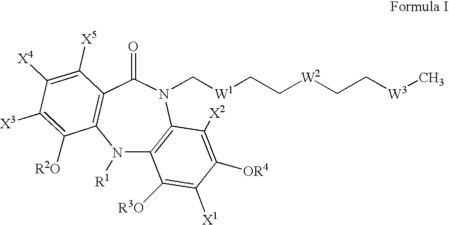

wherein,

W¹, W² and W³ are each independently selected from

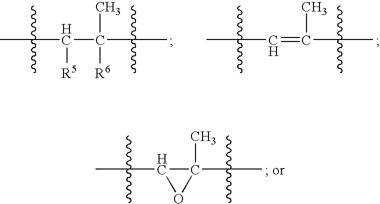

the chain from the tricycle terminates at W³, W² or W¹ with W³, W² or W¹ respectively being either —CH═O, —CH(OC₁₋₆alkyl)₂, —CH₂OH, —CH₂OC₁₋₆alkyl or C(O)OR⁷;

R¹ is selected from the group consisting of H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl, C₃₋₁₀heterocycloalkyl, C(O)H, C(O)C₁₋₁₀alkyl, C(O)C₂₋₁₀alkenyl, C(O)C₂₋₁₀alkynyl, C(O)C₆₋₁₀aryl, C(O)C₅₋₁₀heteroaryl, C(O)C₃₋₁₀cycloalkyl; C(O)C₃₋₁₀heterocycloalkyl and a C-coupled amino acid;

R², R³, and R⁴ are each independently selected from the group consisting of H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl, C₃₋₁₀heterocycloalkyl, C(O)H, C(O)C₁₋₁₀alkyl, C(O)C₂₋₁₀alkenyl, C(O)C₂₋₁₀alkynyl, C(O)C₆₋₁₀aryl, C(O)C₅₋₁₀heteroaryl, C(O)C₃₋₁₀cycloalkyl; C(O)C₃₋₁₀heterocycloalkyl and a C-coupled amino acid;

R⁵ and R⁶ are each independently selected from the group consisting of H, OH, OC₁₋₆alkyl, NH₂, NHC₁₋₆alkyl, N(C₁₋₆alkyl)₂, and NHC(O)C₁₋₆alkyl;

R⁷ is selected from the group consisting of H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl and C₃₋₁₀heterocycloalkyl;

X¹, X², X³, X⁴ and X⁵ are each H; or one of X¹, X², X³, X⁴ or X⁵ is halogen and the remaining ones are H; and wherein, when any of R¹, R², R³, R⁴, R⁵, R⁶ and R⁷ comprises an alkyl, alkenyl, alkynyl, aryl, heteroaryl, cycloalkyl, or heterocycloalkyl group, then the alkyl, alkenyl, alkynyl, aryl, heteroaryl, cycloalkyl, or heterocycloalkyl group is optionally substituted with substituents selected from the group consisting of acyl, amino, acylamino, acyloxy, carboalkoxy, carboxy, carboxyamido, cyano, halo, hydroxyl, nitro, thio, C₁₋₆alkyl, C₂₋₇alkenyl, C₂₋₇alkynyl, C₃₋₁₀cycloalkyl, C₃₋₁₀heterocycloalkyl, C₆₋₁₀aryl, C₅₋₁₀heteroaryl, alkoxy, aryloxy, sulfinyl, sulfonyl, oxo, guanidino and formyl;

and an ester, ether, N-alkylated or N-acylated derivative, or a pharmaceutically acceptable salt, solvate or prodrug thereof.

In further aspect, the invention relates to dibenzodiazepinone analogues and derivatives thereof, represented by Formula II:

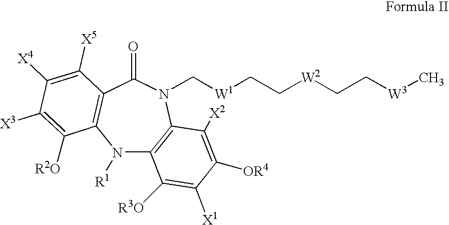

wherein,

W¹, W² and W³ are each independently selected from

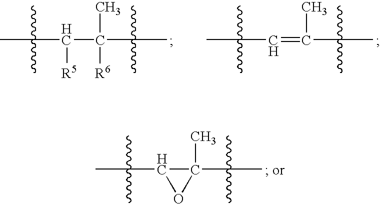

the chain from the tricycle terminates at W³, W² or W¹ with W³, W² or W¹ respectively being either —CH═O, —CH(OC₁₋₆alkyl)₂, —CH₂OH, —CH₂OC₁₋₆alkyl or C(O)OR⁷;

R¹ is selected from the group consisting of H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl, C₃₋₁₀heterocycloalkyl, C(O)H, C(O)C₁₋₁₀alkyl, C(O)C₂₋₁₀alkenyl, C(O)C₂₋₁₀alkynyl, C(O)C₆₋₁₀aryl, C(O)C₅₋₁₀heteroaryl, C(O)C₃₋₁₀cycloalkyl; C(O)C₃₋₁₀heterocycloalkyl and a C-coupled amino acid;

R², R³, and R⁴ are each independently selected from the group consisting of H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl, C₃₋₁₀heterocycloalkyl, C(O)H, C(O)C₁₋₁₀alkyl, C(O)C₂₋₁₀alkenyl, C(O)C₂₋₁₀alkynyl, C(O)C₆₋₁₀aryl, C(O)C₅₋₁₀heteroaryl, C(O)C₃₋₁₀cycloalkyl; C(O)C₃₋₁₀heterocycloalkyl amd a C-coupled amino acid;

R⁵ and R⁶ are each independently selected from the group consisting of H, OH, OC₁₋₆alkyl, NH₂, NHC₁₋₆alkyl, N(C₁₋₆alkyl)₂, and NHC(O)C₁₋₆alkyl;

R⁷ is selected from the group consisting of H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl and C₃₋₁₀heterocycloalkyl;

X¹, X², X³, X⁴ and X⁵ are each H; or one of X¹, X², X³, X⁴ or X⁵ is halogen and the remaining ones are H; and

wherein, when any of R¹, R², R³, R⁴, R⁵, R⁶ and R⁷ comprises an alkyl, alkenyl, alkynyl, aryl, heteroaryl, cycloalkyl, or heterocycloalkyl group, then the alkyl, alkenyl, alkynyl, aryl, heteroaryl, cycloalkyl, or heterocycloalkyl group is optionally substituted with substituents selected from the group consisting of acyl, amino, acylamino, acyloxy, carboalkoxy, carboxy, carboxyamido, cyano, halo, hydroxyl, nitro, thio, C₁₋₆alkyl, C₂₋₇alkenyl, C₂₋₇alkynyl, C₃₋₁₀cycloalkyl, C₃₋₁₀heterocycloalkyl, C₆₋₁₀aryl, C₅₋₁₀heteroaryl, alkoxy, aryloxy, sulfinyl, sulfonyl, oxo, guanidino and formyl;

with the proviso that when W¹, W² and W³ are all —CH═C(CH₃)—, and R², R³ and R⁴ are all H, then R¹ is not H;

and an ester, ether, N-alkylated or N-acylated derivative, or a pharmaceutically acceptable salt, solvate or prodrug thereof.

In one embodiment, R¹ is H, and all other groups are as previously disclosed. In another embodiment, R¹ is —CH₃, and all other groups are as previously disclosed. In another embodiment, R¹ is C₁₋₁₀alkyl, and all other groups are as previously disclosed. In a subclass of this embodiment, the alkyl group is optionally substituted with a substituent selected from halo, fluoro, C₆₋₁₀aryl, and C₅₋₁₀heteroaryl. In another embodiment, R¹ is —C(O)C₁₋₁₀alkyl, and all other groups are as previously disclosed. In another embodiment, R² is H, and all other groups are as previously disclosed. In another embodiment, R³ is H, and all other groups are as previously disclosed. In another embodiment, R⁴ is H, and all other groups are as previously disclosed. In another embodiment, R², R³ and R⁴ are each H, and all other groups are as previously disclosed. In another embodiment, one of R², R³ and R⁴ is CH₃, the others being each H, and all other groups are as previously disclosed. In another embodiment, two of R², R³ and R⁴ are CH₃, the other being H, and all other groups are as previously disclosed. In another embodiment, R², R³ and R⁴ are each CH₃, and all other groups are as previously disclosed. In another embodiment, R², R³ and R⁴ are each H, and W¹ is —CH═C(CH₃)—, and all other groups are as previously disclosed. In another embodiment, R², R³ and R⁴ are each H, and W² is —CH═C(CH₃)—, and all other groups are as previously disclosed. In another embodiment, R², R³ and R⁴ are each H, and W³ is —CH═C(CH₃)—, and all other groups are as previously disclosed. In another embodiment, R¹ is H and R², R³ and R⁴ are each H, and all other groups are as previously disclosed. In another embodiment, R¹ is H, each of W¹, W², and W³ is —CH═C(CH₃)—, and all other groups are as previously disclosed. In another embodiment, R¹ is H, each of W¹, W², and

W³ is —CH₂CH(CH₃)—, and all other groups are as previously disclosed. In another embodiment, X¹ is Br, and each of X², X³, X⁴ and X⁵ are H, and all other groups are as previously disclosed. In another embodiment, if each of W¹, W² and W³ are —CH═C(CH₃)—, and each of R², R³, and R⁴ are H, then R¹ is not H. In further embodiment, if each of W¹, W² and W³ are —CH═C(CH₃)—, and each of R², R³, and R⁴ are H, then R¹ is not CH₃. In further embodiment, if each of W¹, W² and W³ are —CH═C(CH₃)—, and each of R², R³, and R⁴ are H, then R¹ is neither H nor CH₃. The invention encompasses all esters, ethers, N-alkylated or N-acylated derivatives, and pharmaceutically acceptable salts, solvates and prodrugs of the foregoing compounds.

The following are exemplary compounds of the invention, such named compounds are not intended to limit the scope of the invention in any way:

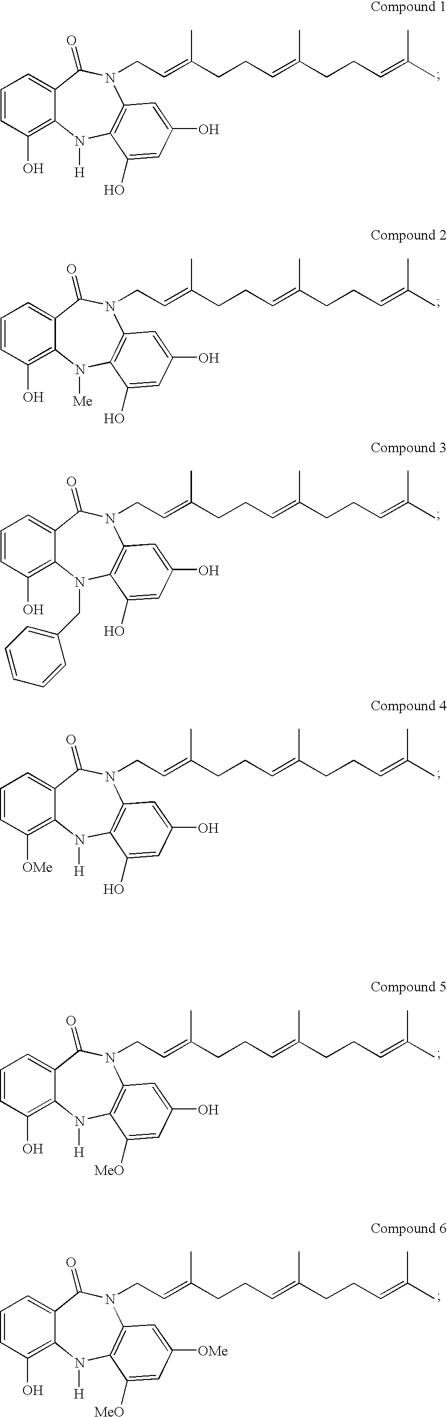

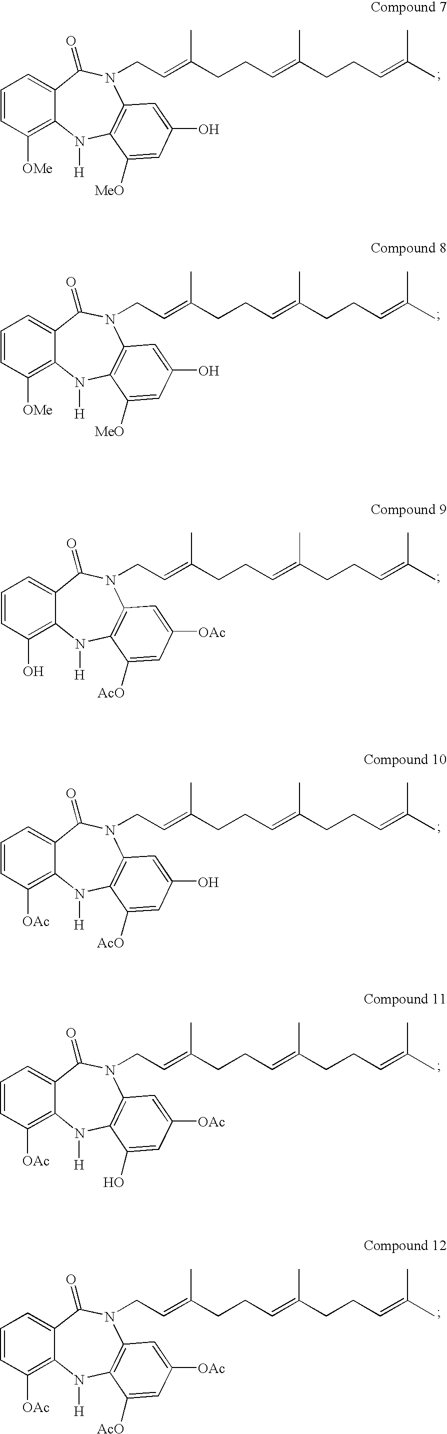

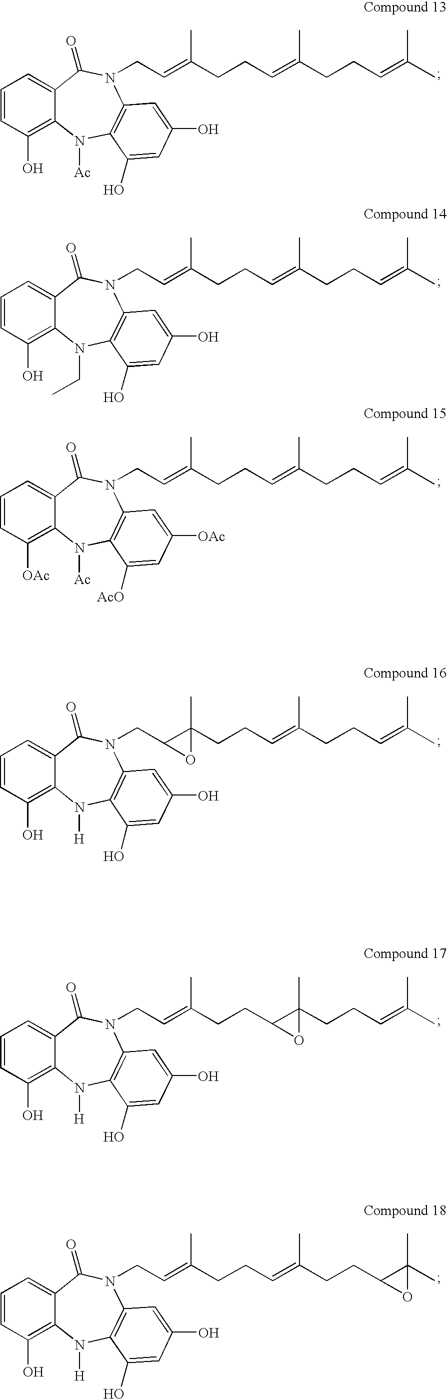

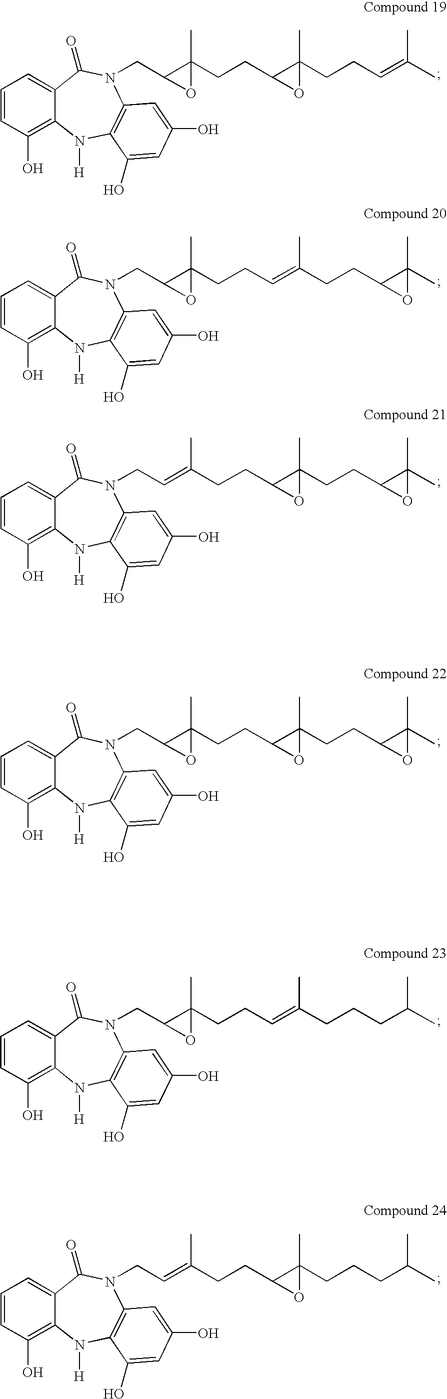

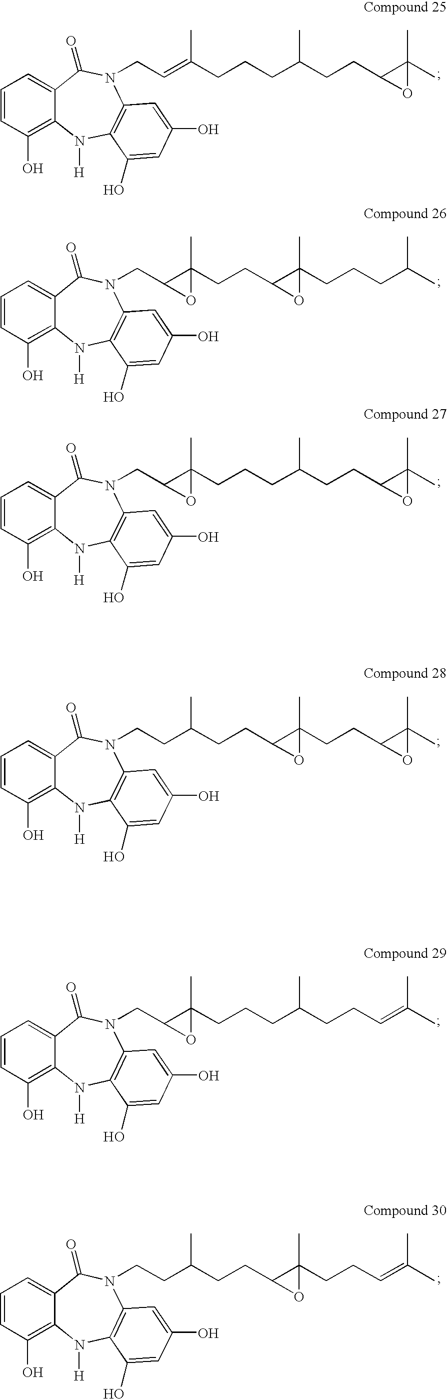

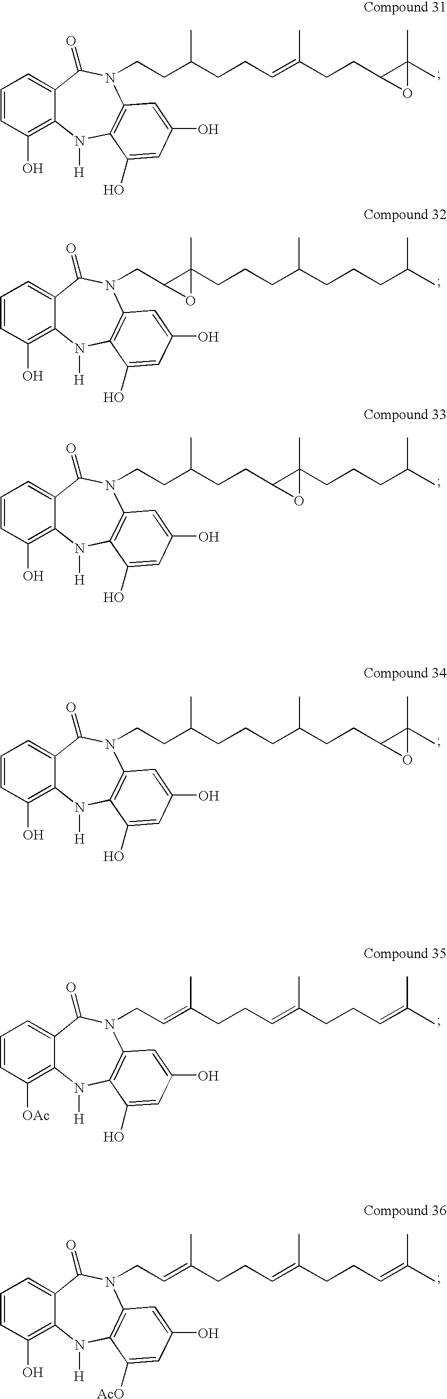

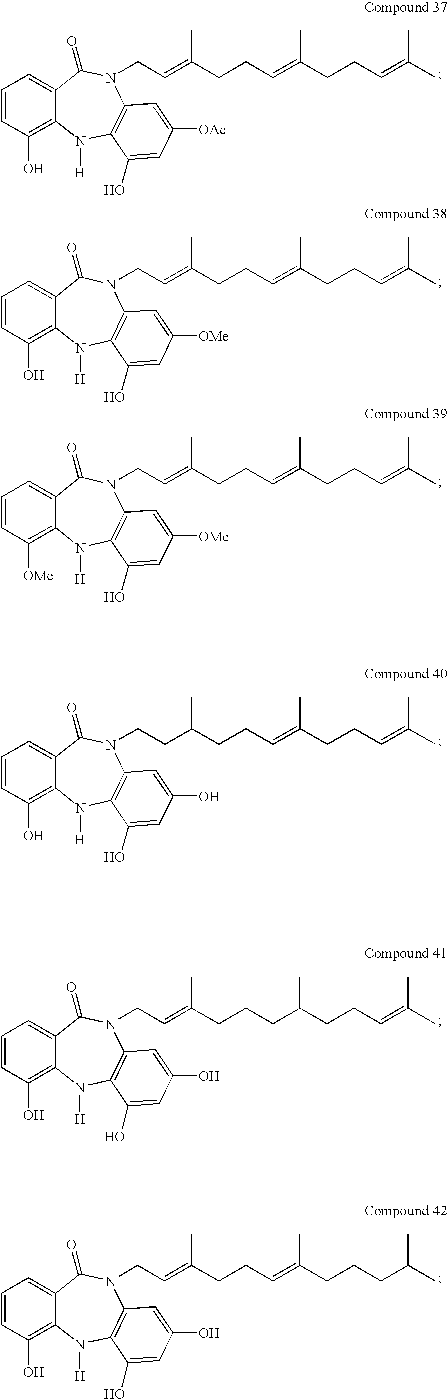

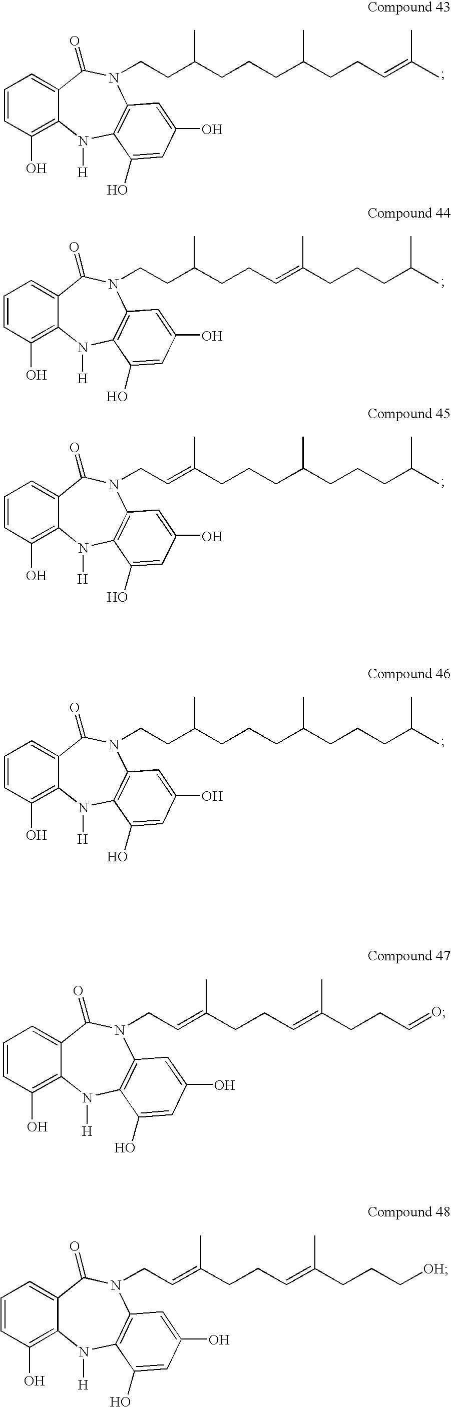

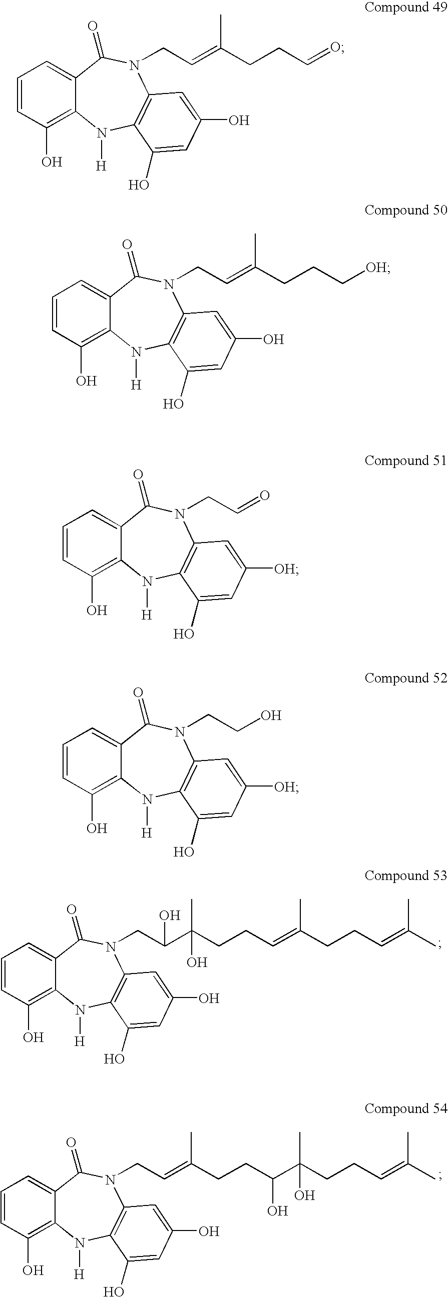

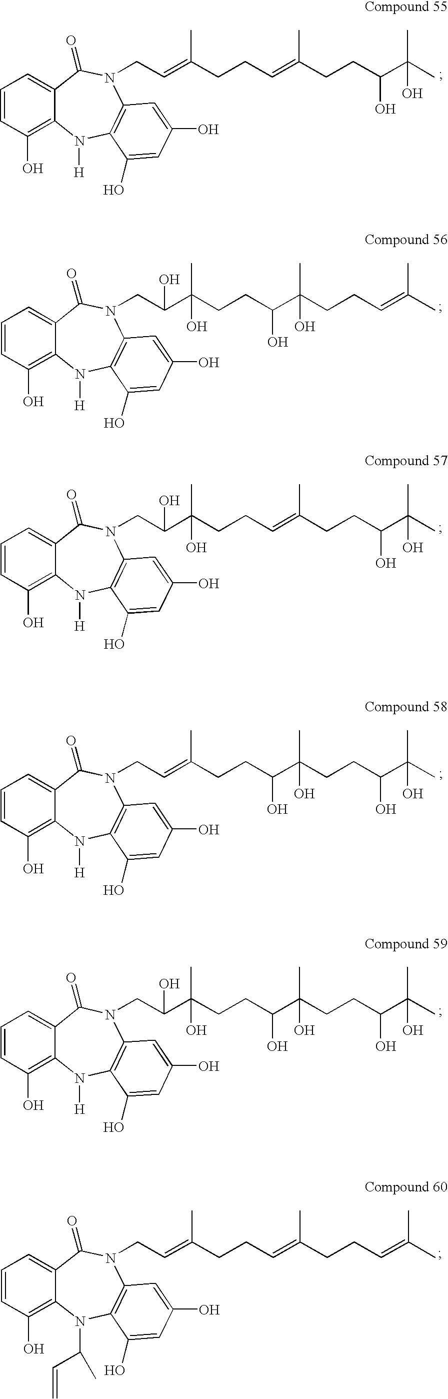

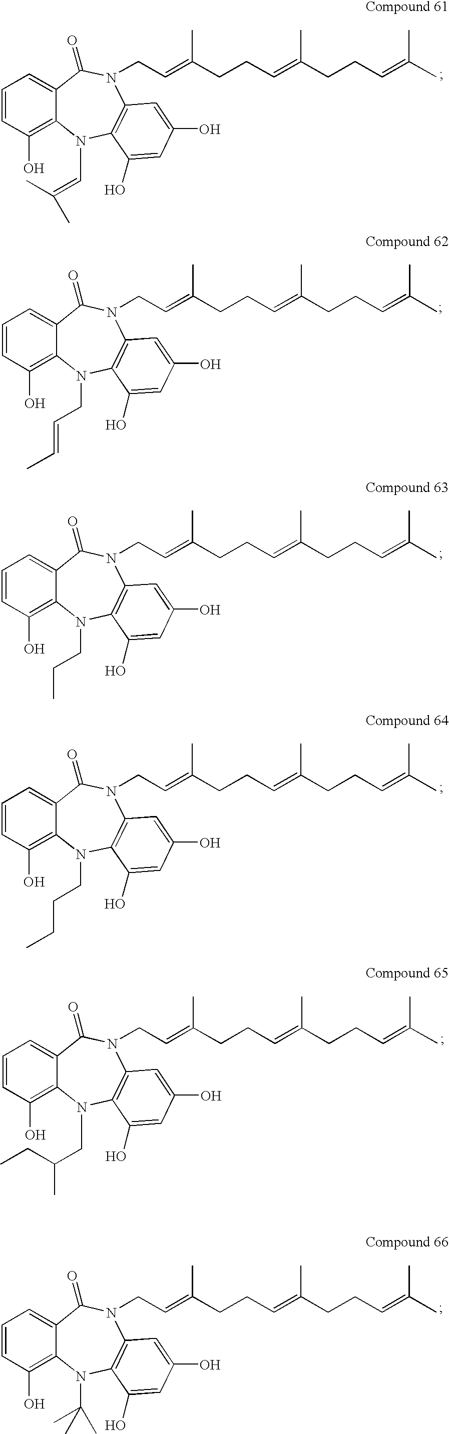

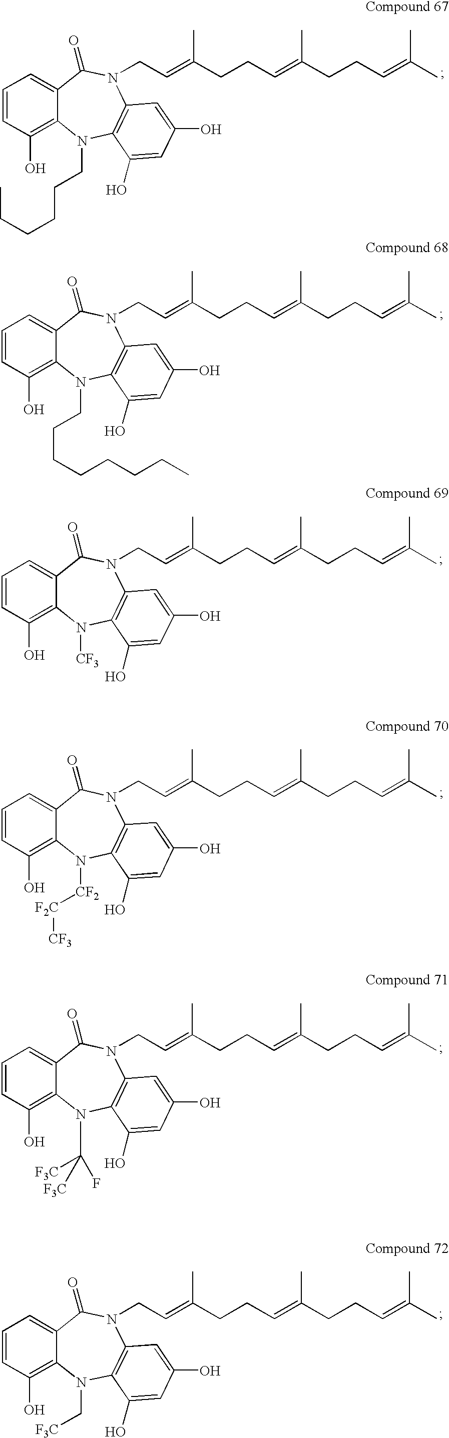

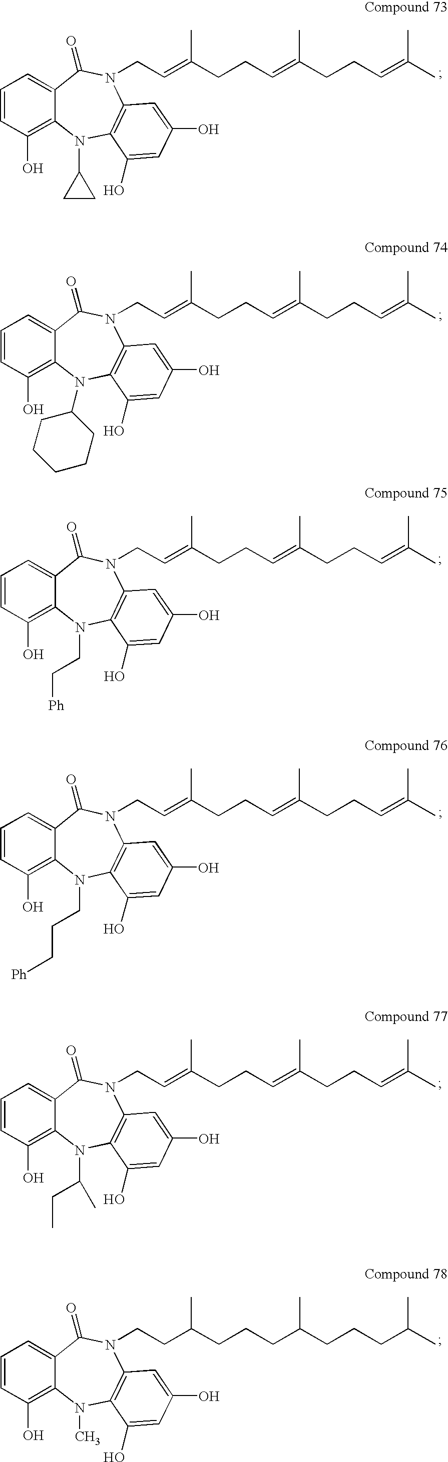

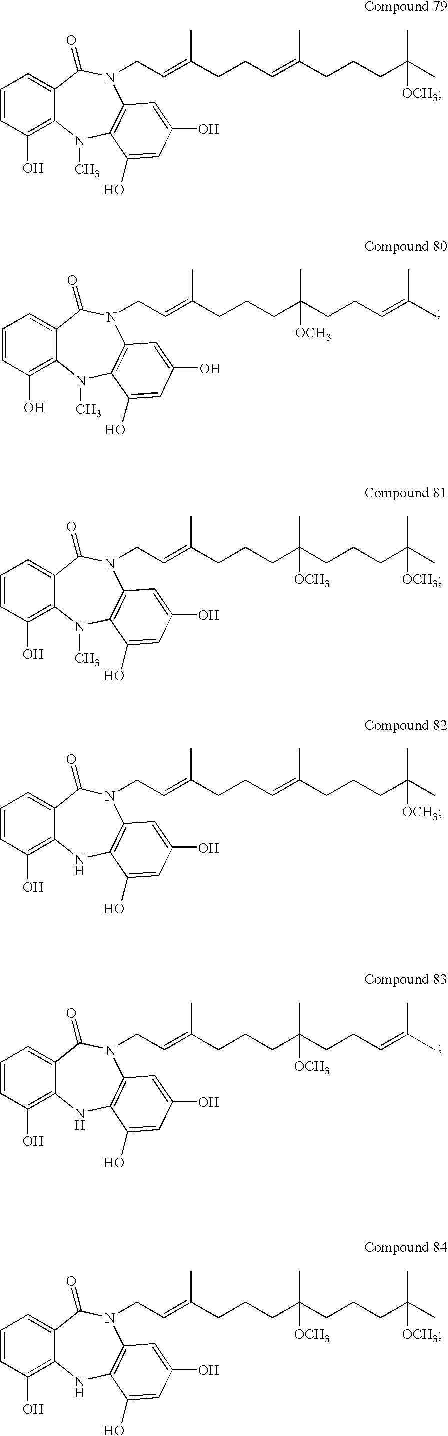

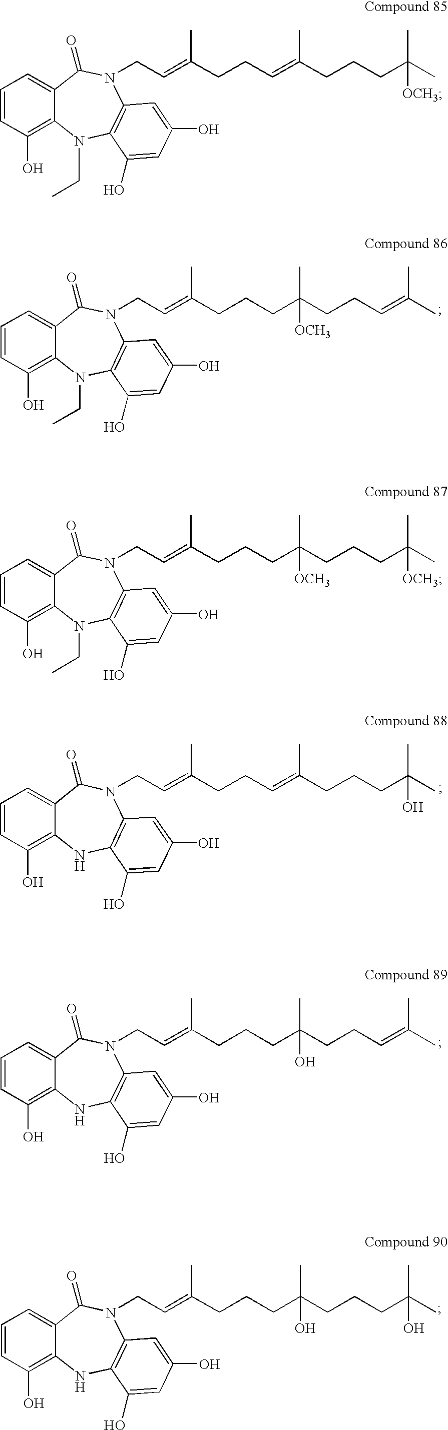

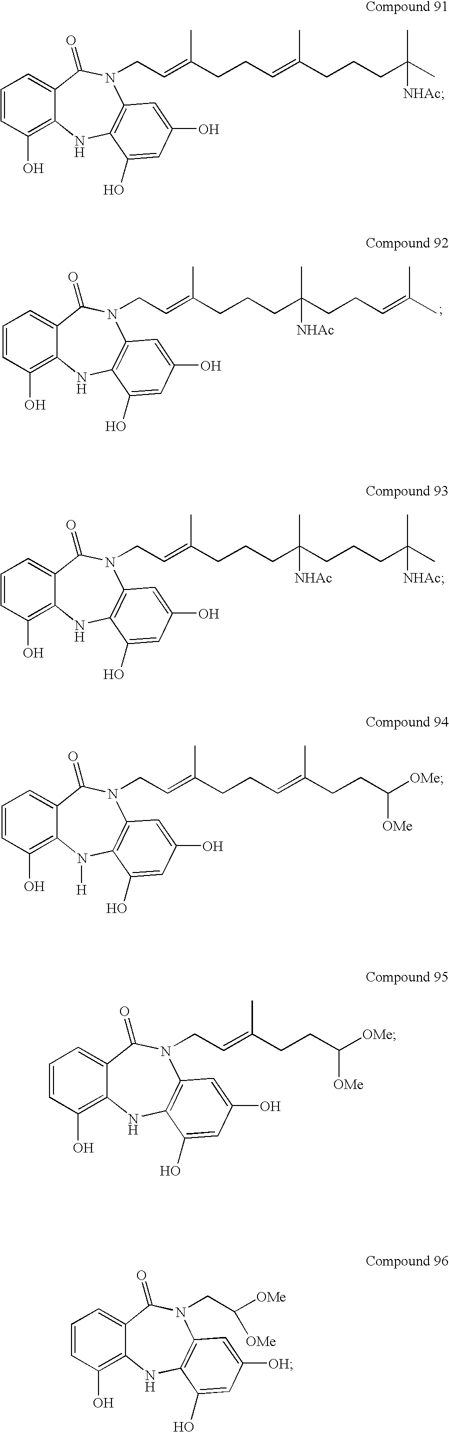

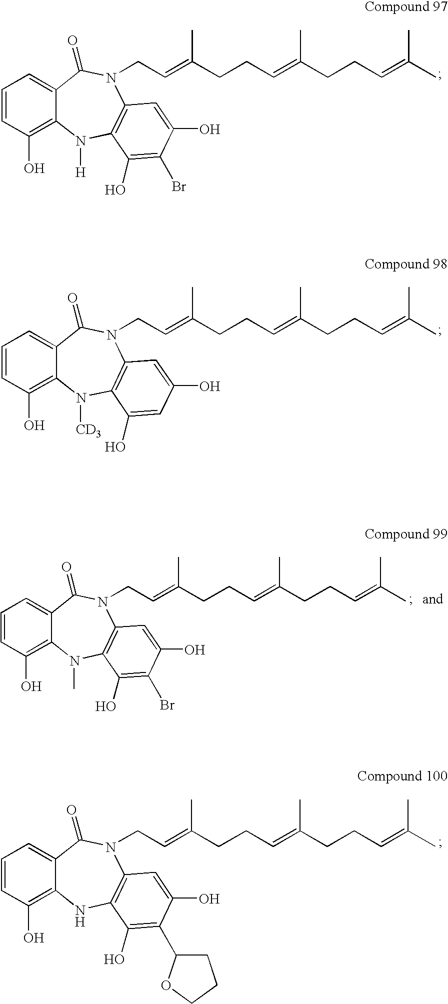

and pharmaceutically acceptable salts, solvates and prodrugs of any one of Compounds 1 to 100.

The invention further provides ethers, esters, N-acylated and N-alkylated derivatives of any of the foregoing Compounds 1-100, as well as pharmaceutically acceptable salts, solvates and prodrugs thereof.

Certain embodiments expressly exclude one or more of the compounds of Formula I. In one embodiment, Compound 1 is excluded. In another embodiment, Compound 2 is excluded. In a further embodiment, both Compound 1 and Compound 2 are excluded.

Prodrugs of the compounds of Formula I or II include compounds wherein one or more of the 4, 6 and 8-hydroxy groups, or any other hydroxyl group on the molecule is bounded to any group that, when administered to a mammalian subject, is cleaved to form the free hydroxyl group. Examples of prodrugs include, but are not limited to, acetate, formate, hemisuccinate, benzoate, dimethylaminoacetate and phosphoryloxycarbonyl derivatives of hydroxy functional groups; dimethylglycine esters, aminoalkylbenzyl esters, aminoalkyl esters or carboxyalkyl esters of hydroxy functional groups. Carbamate and carbonate derivatives of the hydroxy groups are also included. Derivatizations of hydroxyl groups also encompassed, are (acyloxy)methyl and (acyloxy)ethyl ethers, wherein the acyl group contains an alkyl group optionally substituted with groups including, but not limited to, ether, amino and carboxylic acid functionalities, or where the acyl group is an amino acid ester. Also included are phosphate and phosphonate esters, sulfate esters, sulfonate esters, which are in alkylated (such as bis-pivaloyloxymethyl (POM) phosphate triester) or in the salt form (such as sodium phosphate ester (—P(O)O⁻₂Na⁺₂)). For further examples of prodrugs used in anticancer therapy and their metabolism, see Rooseboom et al (2004) Phamacol. Rev vol 56, pp 53-102. When the prodrug contains an acidic or basic moiety, the prodrug may also be prepared as its pharmaceutically acceptable salt.

The compounds of this invention may be formulated into pharmaceutical compositions comprised of a compound of Formula I or II, in combination with a pharmaceutically acceptable carrier, as discussed in Section IV below.

### III. METHODS OF PRODUCING DIBENZODIAZEPINONE ANALOGUES

A. Fermentation

The terms “farnesyl dibenzodiazepinone-producing microorganism”, “producer of farnesyl dibenzodiazepinone,” “dibenzodiazepinone analogue-producing microorganism” and “producer of dibenzodiazepinone analogues,” are used interchangeably to refer to a microorganism that carries genetic information necessary to produce farnesyl dibenzodiazepinone and dibenzodiazepinone analogue compounds, whether or not the organism naturally produces the compounds. The terms apply equally to organisms in which the genetic information to produce the compounds is found in the organism as it exists in its natural environment, and to organisms in which the genetic information is introduced by recombinant techniques.

Specific organisms contemplated herein include, without limitation, organisms of the family Micromonosporaceae, of which preferred genera include Micromonospora, Actinoplanes and Dactylosporangium; the family Streptomycetaceae, of which preferred genera include Streptomyces and Kitasatospora; the family Pseudonocardiaceae, of which preferred genera are Amycolatopsis and Saccharopolyspora; and the family Actinosynnemataceae, of which preferred genera include Saccharothrix and Actinosynnema; however the terms are intended to encompass all organisms containing genetic information necessary to produce farnesyl dibenzodiazepinone and dibenzodiazepinone analogue compounds. A preferred producer of such compounds includes microbial strain 046-ECO11 or [S01]046, a deposit of which was made respectively on Mar. 7, 2003 and Dec. 23, 2003, with the International Depositary Authority of Canada (IDAC), Bureau of Microbiology, Health Canada, 1015 Arlington Street, Winnipeg, Manitoba, Canada R3E,3R2, respectively under Accession No. IDAC 070303-01 and 231203-01.

In one embodiment, ECO-4601 is obtained by cultivating strains of Micromonospora, namely Micromonospora sp. strains 046-ECO11 or [S01]046. Strains 046-ECO11 and [S01]046 were deposited on Mar. 7, 2003, with the International Depositary Authority of Canada (IDAC), Bureau of Microbiology, Health Canada, 1015 Arlington Street, Winnipeg, Manitoba, Canada R3E,3R2, respectively under Accession Nos. 070303-01 and 231203-01. The deposit of the strain was made under the terms of the Budapest Treaty on the International Recognition of the Deposit of Microorganisms for Purposes of Patent Procedure. The deposited strains will be irrevocably and without restriction or condition released to the public upon the issuance of a patent. The deposited strains are provided merely as convenience to those skilled in the art and are not an admission that a deposit is required for enablement.

It is to be understood that the present invention is not limited to use of the particular strains 046-ECO11 and [S01]046. Rather, the present invention contemplates the use of other ECO-4601 producing organisms, such as mutants or variants of 046-ECO11 or [S01]046 that can be derived from this organism by known means such as X-ray irradiation, ultraviolet irradiation, treatment with nitrogen mustard, phage exposure, antibiotic resistance selection and the like; or through the use of recombinant genetic engineering techniques. For examples, see Manual of Industrial Microbiology and biotechnology, Demain and Solomon (1987) American Society for Microbiology; Hesketh et al. (1997) J. Antibiotics 50, no 6, pp 532-535; and Hosoya et al. (1998) Antimicrobial Agents and Chemotherapy, 42, no 8, pp 2041-2047).

The compounds of the present invention may be biosynthesized by various microorganisms. Microorganisms that may synthesize the compounds of the present invention include but are not limited to bacteria of the order Actinomycetales, also referred to as actinomycetes. Non-limiting examples of members belonging to the genera of Actinomycetes include Nocardia, Geodermatophilus, Actinoplanes, Micromonospora, Nocardioides, Saccharothrix, Amycolatopsis, Kutzneria, Saccharomonospora, Saccharopolyspora, Kitasatospora, Streptomyces, Microbispora, Streptosporangium, and Actinomadura. The taxonomy of actinomycetes is complex and reference is made to Goodfellow, Suprageneric Classification of Actinomycetes (1989); Bergey's Manual of Systematic Bacteriology, Vol. 4 (Williams and Wilkins, Baltimore, pp. 2322-2339); and to Embley and Stackebrandt, “The molecular phylogeny and systematics of the actinomycetes,” Annu. Rev. Microbiol. (1994) 48:257-289, for genera that may synthesize the compounds of the invention.

Farnesyl dibenzodiazepinone-producing microorganisms are cultivated in culture medium containing known nutritional sources for actinomycetes. Such media having assimilable sources of carbon, nitrogen, plus optional inorganic salts and other known growth factors, at a pH of about 6 to about 9. Suitable media include, without limitation, the growth media provided in Table 1. Microorganisms are cultivated at incubation temperatures of about 18° C. to about 40° C. for about 3 to about 40 days.

TABLE 1
Examples of Fermentation Media for Compound 1 Production

| Component | QB | MA | KH | RM | JA | FA | XX | CL |
|---|---|---|---|---|---|---|---|---|
| pH*¹ | 7.2 | 7.5 | 7 | 6.85 | 7.3 | 7.0 | 7.0 | 7.0 |
| Glucose | 12 | | 10 | 10 | | 10 | | |
| Sucrose | | | | 100 | | | | |
| Cane molasses | | | | | | 15 | | |
| Corn starch | | | | | 30 | | | |
| Soluble starch | 10 | 25 | | | | | | |
| Potato dextrin | | | 20 | | | 40 | 20 | 20 |
| Corn steep solid | 5 | | | | | | | |
| Corn steep liquor | 5 | | | | 15 | | | |
| Dried yeast | | 2 | | | | | | |
| Yeast extract | | | 5 | | | | 8.34 | |
| Malt extract | | | | | 35 | | | |
| Pharmamedia ™ | 10 | | | | 15 | | | |
| Glycerol | | | | | | | 30 | 20 |
| NZ-Amine A | | | 5 | | | 10 | | |
| Soybean powder | | 15 | | | | | | |
| Fish meal | | | | | | | | 10 |
| Bacto-peptone | | | | | | | 2.5 | 5 |
| MgSO₄•7H₂O | | | | | | 1 | | |
| CaCO₃ | | 4 | 1 | | 2 | 2 | 3 | 2 |
| NaCl | | 5 | | | | | | |
| (NH₄)₂SO₄ | | 2 | | | | | | 2 |
| K₂SO₄ | | | | 0.25 | | | | |
| MgCl₂•6H₂O | | | | 10 | | | | |
| Na₂HPO₄ | | | | | | 3 | | |
| Casamino acid | | | | 0.1 | | | | |
| Proflo oil ™ (mL/L) | 4 | | | | | | 0.05 | |
| Silicon defoamer | | | | | | | 0.3 | |
| oil (mL/L) | | | | | | | | |
| MOPS | | | | 21 | | | | |
| Trace element | 2 | | | | | | | |
| solution*² ml/L | | | | | | | | |

Unless otherwise indicated all the ingredients are in g/L.
*¹The pH is to adjusted as marked prior to the addition of CaCO₃.
*²Trace elements solution contains: ZnCl₂ 40 mg; FeCl₃6H₂O (200 mg); CuCl₂2H₂O (10 mg); MnCl₂•4H₂O; Na₂B₄O₇•10H₂O (10 mg); (NH₄)₆MO₇O₂₄•4H₂O (10 mg) per litre.

The culture media inoculated with a farnesy dibenzodiazepinone-producing microorganism may be aerated by incubating the inoculated culture media with agitation, for example, shaking on a rotary shaker, a shaking water bath, or in a fermentor. Aeration may also be achieved by the injection of air, oxygen or an appropriate gaseous mixture to the inoculated culture media during incubation. Following cultivation, the resulting compounds can be extracted and isolated from the cultivated culture media by techniques known to a person skilled in the art and/or disclosed herein, including for example centrifugation, chromatography, adsorption, filtration. For example, the cultivated culture media can be optionally acidified and mixed with a suitable organic solvent such as methanol, ethanol, n-butanol, ethyl acetate, n-butyl acetate or 4-methyl-2-pentanone. The organic layer can be separated from the mycelial cake for example, by centrifugation and decantation or filtration. The mycelial cake is further optionally extracted with an organic solvent, and the organic extracts combined. The organic layer is further optionally treated, for example by: aqueous washings, precipitation, filtration and the like, followed the removal of the solvent, for example, by evaporation to dryness under vacuum. The resulting residue can optionally be reconstituted with for example water, ethyl ether, ethanol, ethyl acetate, methanol or a mixture thereof, and re-extracted in a two-phase system with a suitable organic solvent such as hexane, carbon tetrachloride, methylene chloride or a mixture thereof. After removal of the solvent, the compound can be further purified by the use of standard techniques such as normal and reverse-phase liquid chromatography, crystallization, sublimation, adsorption, mass exclusion chromatography, and the like.

B. Chemical Modifications

The farnesyl dibenzodiazepinone Compound 1 is biosynthesized by microorganisms and isolated as described herein, and in Canadian patent 2,466,340. Compound 1 is subjected to random and/or directed chemical modifications to form compounds that are derivatives or structural analogues. Such derivatives or structural analogues having similar functional activities are within the scope of the present invention. The farnesyl dibenzodiazepinone may be modified by one or more chemical modification steps, using methods known in the art and described herein. Examples of chemical modifications procedures are also provided in Examples 4 to 9 and Example 15.

Dibenzodiazepinone analogues of Compound 1, for example those identified herein as the compounds of Formula II and Compounds 2 to 100, are generated by standard organic chemistry approaches. General principles of organic chemistry required for making and manipulating the compounds described herein, including functional moieties, reactivity and common protocols are described, for example, in “Advanced Organic Chemistry,” 4^{th} Edition by Jerry March (1992), Wiley-Interscience, USA. In addition, it will be appreciated by one of ordinary skill in the art that the synthetic methods described herein may use a variety of protecting groups, whether or not they are explicitly described. A “protecting group” as used herein means a moiety used to block one or more functional moieties such as reactive groups including oxygen, sulfur or nitrogen, so that a reaction can be carried out selectively at another reactive site in a polyfunctional compound. General principles for the use of protective groups, their applicability to specific functional groups and their uses are described for example in T. H. Greene and P. G. M. Wuts, Protective Groups in Organic Synthesis, 3^{rd} Edition, John Wiley & Sons, New York (1999).

Alcohols and phenols are protected with, for example: silyl ethers (TMS: trimethylsilyl, TIPS: triisopropylsilyl), acetals (MOM: methyloxymethyl, BOM: benzyloxymethyl), esters (acetate, benzoyl) and ethers (Bn: benzyl). Alcohols are deprotected by conditions such as: TBAF (tetrabutylammonium fluoride) for silyl ethers, aqueous acid catalysis for acetals and esters, saponification for esters, and hydrogenolysis for Bn and BOM. Amine is protected using standard amino acid protecting groups, for example, carbamates (such as t-butyl (BOC) and benzyl (CBZ)), fluorene derivatives (such as FMOC: N-(9-fluorenylmethoxycarbonyl)-), etc. Amine is deprotected by conditions such as: acid hydrolysis for BOC, hydrogenolysis for CBZ, or base treatment for FMOC. All protection and deprotection conditions are demonstrated in the Greene et al reference above.

Those skilled in the art will readily appreciate that many synthetic chemical processes may be used to produce analogues of Compound 1. The following schemes are exemplary of the routine chemical modifications that may be used to produce compounds of Formula II. Any chemical synthetic process known to a person skilled in the art providing the structures described herein may be used and are therefore comprised in the present invention.

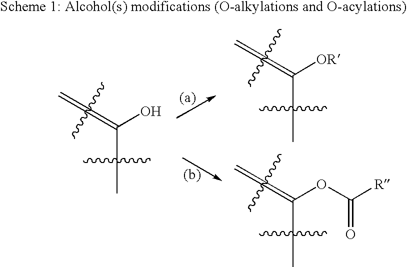

wherein, R′ and R″ are each selected from alkyl, alkenyl, alkynyl, cycloalkyl, heterocycloalkyl, aryl, or heteroaryl; or R″C(O)— is HC(O)— or a C-coupled amino acid.

In Scheme 1, Phenols in positions 4, 6 and 8 (for position numbers, see Example 3) are independently alkylated (to produce an ether) or acylated (to produced an ester). In Scheme 1 (a), allylation is accomplished with an alkylating agent such as R′X is a diazoalkane, or with a R′X reagent, wherein X is a suitable leaving group such as Br, I and trifluoromethane sulfonate in the presence of a base, preferably, a diazoalkane is used. When R′ is aryl or heteroaryl, the reaction may further need the use of a catalyst, such as copper salts (Ullman ether synthesis, Jerry March, supra). In Scheme 1 (b), a phenolic alcohol is converted to ester when reacted with an activated carboxylic acid (R″C(O)X) such as an acid halide, anhydride, N-hydroxysuccinimide ester, or a carboxylic acid activated by a coupling agent (e.g.: EDC (1-(3-dimethylaminopropyl)-3-disopropylethylcarbodiimide hydrochloride); or HATU (O-(7-azabenzotriazol-1-yl)-N,N, N′,N′tetramethyluronium hexafluorophosphate)) with a base (e.g., pyridine or N,N-diisopropylethylamine (DIPEA)) and optional acatalysts such HOBt (1-hydroxybenzotriazole hydrate) and/or DMAP (4-(dimethylamino)pyridine). The same reactions may be accomplished on alcohols formed by farnesyl modification reactions (Scheme 3).

Scheme 1 is used to obtain, for example, Compounds 4 to 12 and 35 to 39 from Compound 1, and Compound 15 from Compound 13; and to produce any of the Compounds of Formula I or II comprising an O-alkyl or O-acyl group.

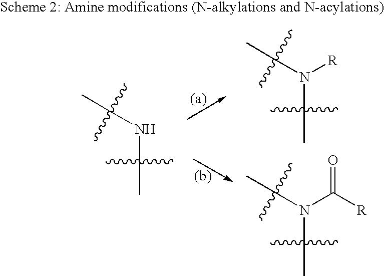

wherein, R is selected from alkyl, alkenyl, alkynyl, cycloalkyl, heterocycloalkyl, aryl, and heteroaryl; or RC(O)— is HC(O)— or a C-coupled amino acid.

In Scheme 2, amine group in position 5 (for position, see Example 3) is optionally alkylated or acylated. In Scheme 2(a), an amine is alkylated using an R^{x} alkylating agent such as dialkyl sulfates and alkyl halides, preferably in the presence of a base (e.g., sodium bicarbonate, pyridine and the like). When R is an aryl or a heteroaryl group, the alkylation reaction with an aryl iodide may further need a catalyst, such as copper (for an example, see Plater et al. 2000, J. Chem. Soc., Perkin Trans. 1, 2695-2701). In Scheme 2(b), an amine is acylated when reacted with an activated carboxylic acid such as an acid halide, anhydride, N-hydroxysuccinimide ester, or a carboxylic acid activated by a coupling agent (see Scheme 1) in the presence of a base like DIPEA, and optional use of a catalyst, such as DMAP or HOBt.

Scheme 2 is used to prepare, for example, Compounds 2, 3, 13, 14, 60 to 77 and 98 from Compound 1, and Compound 78 from Compound 46; and to produce any of the Compounds of Formula I or II comprising an N-alkyl or N-acyl group.

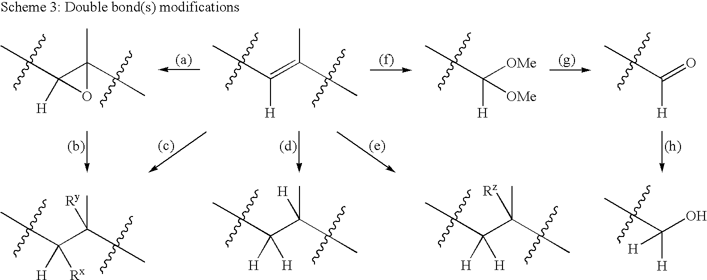

wherein R^{x} and R^{y} are each selected from H, OH and OC₁₋₆alkyl, provided that at least one of R^{x} or R^{y} is OH; R^{z} is selected from halogen, OH, OC₁₋₆alkyl, and NHC(O)C₁₋₆ alkyl.

In Scheme 3, double bond is modified by: (a) epoxidation; (b) epoxide ring opening (dihydroxylation, hydration, or hydroxyalkoxylation product) (c) direct dihydroxylation, hydration, or hydroxyalkoxylation; (d) hydrogenation; (e) electrophilic addition; (f) ozonolysis; (g) hydrolysis of the acetal produced in (f); and (h) reduction of the aldehyde produced in (g). In (a), epoxides are obtained from the reaction of double bonds with oxidizing agents such as peracids (e.g., mCPBA: 4-chloroperbenzoic acid). In (b), the epoxide obtained in (a) is opened by nucleophiles. In basic conditions epoxides will preferentially open to give the residual OH at the most hindered position (R^{y}). In acidic conditions, the compound having the residual OH at the R^{x} position will be formed as the major product. In (b), the diol (dihydroxylation product: R^{x} and R^{y} are each OH) is obtained from hydrolysis of the epoxide in acidic or basic aqueous conditions, preferably acidic. In (b), also alcoholysis of the epoxide (hydroxyalkoxylation product) is accomplished in basic (R^{x} is OC₁₋₆alkyl and R^{y} is OH, as major) or acidic (R^{x} is OH and R^{y} is OC₁₋₆alkyl, as major) conditions in a C₁₋₆alkyl alcohol as solvent, preferably acidic conditions. In (b), hydration product (R^{x} is H and R^{y} is OH, as the major component) is obtained from the opening of the epoxide by a hydride source (e.g. lithium aluminium hydride (LAH)). In (c), the diol (R^{x} and R^{y} are each OH) is obtained from the dihydroxylation of the double bond in oxidizing conditions (e.g.: osmium tetroxide, potassium permanganate, N-methylmorpholine-N-oxide, and the like). In (c), hydration product (R^{x} is OH and R^{y} is H, as major) is obtained from the oxidative cleavage (NaOH/hydrogen peroxide) of the intermediate formed by hydroboration of the double bond (e.g., using 9-BBN (9-borabicyclo[3,3,1]nonane), and the like). In (d), hydrogenation is carried out using a hydrogen source (e.g. hydrogen, formic acid) and a catalyst (such as rhodium, platinum, or palladium). In (e), electrophilic addition to the double bond is achieved by the formation of a carbocation from addition of a proton in acidic conditions (e.g., p-toluene sulfonic acid, alkyl sulfate/NaHCO₃/MeOH, and the like), and trapping of the carbocation with an alcohol (C₁₋₆alkyl alcohol, hydroalkoxylation), water (hydration) or another electron rich atom (e.g., an halogen or a nitrile, which is subsequently hydrolyzed to give an amide). In (f), an acetal is obtained by the reaction of the double bond with a controlled quantity of ozone and the use of a dialkyl sulfide (e.g., Me₂S) to decompose the ozonide at the end. When the ozonolysis is done in an alcohol, e.g. methanol, the dialkyl actetal is obtained. In (g), an aldehyde is obtained by the hydrolysis of the acetal obtained in (f). In (h), the aldehyde obtained from (g) is reduced to alcohol by a reducing agent [H] such as sodium borohydride (NaBH₄), sodium cyanoborohydride (NaBH₃CN) or LAH.

Scheme 3 is used to obtain, for example: in (a) Compounds 16 from Compound 1, and Compounds 23, 24 and 26 from Compound 42, Compounds 25, 27 and 29 from Compound 41, Compounds 28, 30 and 31 from Compound 40, and Compounds 32, 33 and 34 respectively from Compounds 45, 44 and 43; in (b)

Compounds 53 to 59 respectively from Compounds 16 to 22; in (c) Compounds 53 to 59 from Compound 1; in (d) Compounds 40 to 46 from Compound 1, and Compound 78 from Compound 2; in (e) Compounds 79 to 81 from Compound 2, Compounds 82 to 84 and 88 to 93 from Compound 1, Compounds 85 to 87 from Compound 14; in (f) Compounds 94 to 96 from Compound 1; in (g) Compounds 47, 49 and 51 from Compound 1; and in (h) Compounds 48, 50 and 52 respectively from Compounds 47, 49 and 51. Schemes 3 (a)-(g) are also used to produce any Compound of Formula I or II comprising a modified farnesyl group.

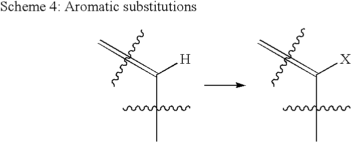

wherein, X is selected from F, Cl, Br and I.

In Scheme 4, the aryl group is modified (when one of X¹ to X⁵ is halo in Formula I or II) by aromatic substitutions, such as halogenation, including bromination, chlorination, fluorination, and iodination. Halogenating agents include bromine, N-haloamides (e.g, N-bromosuccinimide (NBS), tetraalkylammonium polyhalides), chlorine, chlorinated cyclohexadienes, N-chloroamines, chlorodimethylsulfonium chloride, sulfur monochloride/aluminum chloride/thionyl chloride, iodine chloride, iodine/oxidizing agent (e.g, nitric acid, iodic acid, sulfur trioxide, etc), silver(II) fluoride, cesium fluoroxysulfate, and the like.

Scheme 4 is used to prepare, for example, Compound 97 from Compound 1; and to produce any of the Compounds of Formula I or II comprising a halogen group on the aromatic ring.

Prodrugs are prepared by routine chemical modifications such as described in Jerry March, supra, including esterification and alkylation reactions, i.e., use of activated acids or mixed anhydrides (acyl halides, use of coupling reagents, etc), and by the use of alkylating agents (R-X, wherein X is a leaving group, such as diazo, and R is the desired group). Phosphate prodrugs are prepared by phosphorylation, for example, by a procedure such as described in U.S. Pat. No. 5,561,122 (Pettit et al) and in Hwang and Cole (2004), Org. Lett., vol 6, no 10, 1555-1556 ((POM)₂phosphate triester from (POM)₂phosphoryl chloride).

### IV. PHARMACEUTICAL COMPOSITIONS COMPRISING THE COMPOUNDS OF THE INVENTION

The invention provides a pharmaceutical composition comprising a compound of Formula I or a derivative of a compound of Formula I, or a pharmaceutically acceptable salt, solvate or prodrug thereof, in combination with a pharmaceutically acceptable carrier. The pharmaceutical composition comprising a dibenzodiazepinone analogue or derivative is useful for treating diseases and disorders associated with uncontrolled cellular growth and proliferation, such as a neoplastic condition. The pharmaceutical composition is also useful in treating other diseases and disorders, including inflammation, autoimmune diseases, infections, neurodegenerative diseases and stress. The pharmaceutical composition comprising a dibenzodiazepinone analogue or derivative may be packaged into a convenient commercial package providing the necessary materials, such as the pharmaceutical composition and written instructions for its use in treating a neoplastic condition, in a suitable container.

The analogues and derivatives of the present invention, including their pharmaceutically acceptable salts, solvates and prodrugs, can be formulated for oral, sublingual, intranasal, intraocular, rectal, transdermal, mucosal, topical or parenteral administration for the therapeutic or prophylactic treatment of neoplastic and proliferative diseases and disorders. Parenteral modes of administration include without limitation, intradermal, subcutaneous (s.c., s.q., sub-Q, Hypo), intramuscular (i.m.), intravenous (i.v.), intraperitoneal (i.p.), intra-arterial, intramedulary, intracardiac, intra-articular (joint), intrasynovial (joint fluid area), intracerebral or intracranial, intraspinal, intracisternal, and intrathecal (spinal fluids). Any known device useful for parenteral injection or infusion of drug formulations can be used to effect such administration. For oral and/or parental administration, compounds of the present invention can be mixed with conventional pharmaceutical carriers and excipients and used in the form of solutions, emulsions, tablets, capsules, soft gels, elixirs, suspensions, syrups, wafers and the like. The compositions comprising a compound of the present invention will contain from about 0.1% to about 99.9%, about 1% to about 98%, about 5% to about 95%, about 10% to about 80% or about 15% to about 60% by weight of the active compound.

The pharmaceutical preparations disclosed herein are prepared in accordance with standard procedures and are administered at dosages that are selected to reduce, prevent, or eliminate cancer. (See, e.g., Remington's Pharmaceutical Sciences, Mack Publishing Company, Easton, Pa.; and Goodman and Gilman, Pharmaceutical Basis of Therapeutics, Pergamon Press, New York, N.Y., for a general description of the methods for administering various agents for human therapy).

As used herein, the term “unit dosage” refers to physically discrete units suitable as unitary dosages for human subjects and other mammals, each unit containing a predetermined quantity of dibenzodiazepinone analogue calculated to produce the desired therapeutic effect, in association with a suitable pharmaceutically acceptable carrier. In one embodiment, the unit dosage contains from 10 to 3000 mg of active ingredient. In another embodiment, the unit dosage contains 20 to 1000 mg of active ingredient. The compositions of the present invention can be delivered using controlled (e.g., capsules) or sustained release delivery systems (e.g., bioerodable matrices). Exemplary delayed release delivery systems for drug delivery that are suitable for administration of the compositions of the invention are described in U.S. Pat. Nos. 4,452,775 (issued to Kent), 5,039,660 (issued to Leonard), and 3,854,480 (issued to Zaffaroni).

The pharmaceutically-acceptable compositions of the present invention comprise one or more compounds of the present invention in association with one or more non-toxic, pharmaceutically-acceptable carriers and/or diluents and/or adjuvants and/or excipients, collectively referred to herein as “carrier” materials, and if desired other active ingredients. Pharmaceutically acceptable carriers include, for example, solvents, vehicles or medium such as saline, buffered saline, dextrose, water, glycerol, ethanol, propylene glycol, polysorbate 80 (Tween-80™), poly(ethylene) glycol 300 and 400 (PEG 300 and 400), PEGylated castor oil (E.g. Cremophor EL), poloxamer 407 and 188, hydrophobic carriers, and combinations thereof. Hydrophobic carriers include, for example, fat emulsions, lipids, PEGylated phopholids, polymer matrices, biocompatible polymers, lipospheres, vesicles, particles, and liposomes. The term specifically excludes cell culture medium.

Excipients or additives included in a formulation have different purposes depending, for example on the nature of the drug, and the mode of administration. Examples of generally used excipients include, without limitation: stabilizing agents, solubilizing agents and surfactants, buffers, antioxidants and preservatives, tonicity agents, bulking agents, lubricating agents, emulsifiers, suspending or viscosity agents, inert diluents, fillers, disintegrating agents, binding agents, wetting agents, lubricating agents, antibacterials, chelating agents, sweetners, perfuming agents, flavouring agents, coloring agents, administration aids, and combinations thereof.

The compositions may contain common carriers and excipients, such as cornstarch or gelatin, lactose, sucrose, microcrystalline cellulose, kaolin, mannitol, dicalcium phosphate, sodium chloride and alginic acid. The compositions may contain crosarmellose sodium, microcrystalline cellulose, sodium starch glycolate and alginic acid.

Formulations for parenteral administration can be in the form of aqueous or non-aqueous isotonic sterile injection solutions, suspensions or fat emulsions, comprising a compound of this invention, or a pharmaceutically acceptable salt or prodrug thereof. The parenteral form used for injection must be fluid to the extent that easy syringability exists. These solutions or suspensions can be prepared from sterile concentrated liquids, powders or granules. The compounds can be dissolved in a carrier such as a solvent or vehicle, for example, polyethylene glycol, propylene glycol, ethanol, corn oil, benzyl alcohol, glycofurol, N,N-dimethylacetamide, N-methylpyrrolidone, glycerine, saline, dextrose, water, glycerol, hydrophobic carriers, and combinations thereof.

Excipients used in parenteral preparations also include, without limitation, stabilizing agents (e.g. carbohydrates, amino acids and polysorbates), solubilizing agents (e.g. cetrimide, sodium docusate, glyceryl monooleate, polyvinylpyrolidone (PVP) and polyethylene glycol (PEG)) and surfactants (e.g. polysorbates, tocopherol PEG succinate, poloxamer and Cremophor™), buffers (e.g. acetates, citrates, phosphates, tartrates, lactates, succinates, amino acids and the like), antioxidants and preservatives (e.g. BHA, BHT, gentisic acids, vitamin E, ascorbic acid and sulfur containing agents such as sulfites, bisulfites, metabisulfites, thioglycerols, thioglycolates and the like), tonicity agents (for adjusting physiological compatibility), suspending or viscosity agents, antibacterials (e.g. thimersol, benzethonium chloride, benzalkonium chloride, phenol, cresol and chlorobutanol), chelating agents, and administration aids (e.g. local anesthetics, anti-inflammatory agents, anti-clotting agents, vaso-constrictors for prolongation and agents that increase tissue permeability), and combinations thereof.

Parenteral formulations using hydrophobic carriers include, for example, fat emulsions and formulations containing lipids, lipospheres, vesicles, particles and liposomes. Fat emulsions include in addition to the above-mentioned excipients, a lipid and an aqueous phase, and additives such as emulsifiers (e.g. phospholipids, poloxamers, polysorbates, and polyoxyethylene castor oil), and osmotic agents (e.g. sodium chloride, glycerol, sorbitol, xylitol and glucose). Liposomes include natural or derived phospholipids and optionally stabilizing agents such as cholesterol.

In another embodiment, the parenteral unit dosage form of the compound can be a ready-to-use solution of the compound in a suitable carrier in sterile, hermetically sealed ampoules or in sterile pre-loaded syringes. The suitable carrier optionally comprises any of the above-mentioned excipients.

Alternatively, the unit dosage of the compound of the present invention can be in a concentrated liquid, powder or granular form for ex tempore reconstitution in the appropriate pharmaceutically acceptable carrier at the time of delivery. In addition the above-mentioned excipients, powder forms optionally include bulking agents (e.g. mannitol, glycine, lactose, sucrose, trehalose, dextran, hydroxyethyl starch, ficoll and gelatin), and cryo or lyoprotectants.

For example, in intravenous (IV) use, a sterile formulation of the compound of formula I and optionally one or more additives, including solubilizers or surfactants, can be dissolved or suspended in any of the commonly used intravenous fluids and administered by infusion. Intravenous fluids include, without limitation, physiological saline, phosphate buffered saline, 5% glucose or Ringer's™ solution.

In another example, in intramuscular preparations, a sterile formulation of the compound of the present invention or suitable soluble salts or prodrugs forming the compound, can be dissolved and administered in a pharmaceutical diluent such as Water-for-Injection (WFI), physiological saline or 5% glucose. A suitable insoluble form of the compound may be prepared and administered as a suspension in an aqueous base or a pharmaceutically acceptable oil base, e.g. an ester of a long chain fatty acid such as ethyl oleate.

For oral use, solid formulations such as tablets and capsules are particularly useful. Sustained released or enterically coated preparations may also be devised. For pediatric and geriatric applications, suspension, syrups and chewable tablets are especially suitable. For oral administration, the pharmaceutical compositions are in the form of, for example, tablets, capsules, suspensions or liquid syrups or elixirs, wafers and the like. For general oral administration, excipient or additives include, but are not limited to inert diluents, fillers, disintegrating agents, binding agents, wetting agents, lubricating agents, sweetening agents, flavoring agents, coloring agents and preservatives.

The oral pharmaceutical composition is preferably made in the form of a unit dosage containing a therapeutically-effective amount of the active ingredient. Examples of such dosage units are tablets and capsules. For therapeutic purposes, the tablets and capsules which can contain, in addition to the active ingredient, conventional carriers such as: inert diluents (e.g., sodium and calcium carbonate, sodium and calcium phosphate, and lactose), binding agents (e.g., acacia gum, starch, gelatin, sucrose, polyvinylpyrrolidone (Providone), sorbitol, or tragacanth methylcellulose, sodium carboxymethylcellulose, hydroxypropyl methylcellulose, and ethylcellulose), fillers (e.g., calcium phosphate, glycine, lactose, maize-starch, sorbitol, or sucrose), lubricants or lubricating agents (e.g., magnesium stearate or other metallic stearates, stearic acid, polyethylene glycol, waxes, oils, silica and colloical silica, silicon fluid or talc), disintegrants or disintegrating agents (e.g., potato starch, corn starch and alginic acid), flavouring, coloring agents, or acceptable wetting agents. Carriers may also include coating excipients such as glyceryl monostearate or glyceryl distearate, to delay absorption in the gastrointestinal tract.

Oral liquid preparations, generally in the form of aqueous or oily solutions, suspensions, emulsions, syrups or elixirs, may contain conventional additives such as suspending agents, emulsifying agents, non-aqueous agents, preservatives, coloring agents and flavoring agents. Examples of additives for liquid preparations include acacia, almond oil, ethyl alcohol, fractionated coconut oil, gelatin, glucose syrup, glycerin, hydrogenated edible fats, lecithin, methyl cellulose, methyl or propyl para-hydroxybenzoate, propylene glycol, sorbitol, or sorbic acid.

For both liquid and solid oral preparations, flavoring agents such as peppermint, oil of wintergreen, cherry, grape, fruit flavoring or the like can also be used. It may also be desirable to add a coloring agent to make the dosage form more aesthetic in appearance or to help identify the product. For topical use the compounds of present invention can also be prepared in suitable forms to be applied to the skin, or mucus membranes of the nose and throat, and can take the form of creams, ointments, liquid sprays or inhalants, lozenges, or throat paints. Such topical formulations further can include chemical compounds such as dimethylsulfoxide (DMSO) to facilitate surface penetration of the active ingredient. For application to the eyes or ears, the compounds of the present invention can be presented in liquid or semi-liquid form formulated in hydrophobic or hydrophilic bases as ointments, creams, lotions, paints or powders. For rectal administration the compounds of the present invention can be administered in the form of suppositories admixed with conventional carriers such as cocoa butter, wax or other glyceride.

### V. MEDICAL USE IN THE TREATMENT OF NEOPLASMS

In one aspect, the invention relates to a method for inhibiting growth and/or proliferation of cancer cells in a mammal. In another aspect, the invention provides a method for treating neoplasms in a mammal. Mammals include ungulates (e.g. sheeps, goats, cows, horses, pigs), and non-ungulates, including rodents, felines, canines and primates (i.e. human and non-human primates). In a preferred embodiment, the mammal is a human.

Although not wishing to be bound by any particular theory, dibenzodiazepinone analogues and derivatives of the present invention may exert their anticancer effects, at least in part, through interaction with the peripheral benzodiazepine receptor (PBR). PBR is an evolutionarily conserved 18-kDa protein, which is present in all tissues but highly expressed in steroid producing tissues and cancers, and has been associated with numerous biological functions, including regulation of apoptosis, regulation of cell proliferation, and stimulation of steroidogenesis. PBR is a critical component of the mitochondrial permeability transition pore (MPTP), a multiprotein complex located at the contact site between inner and outer mitochondrial membranes, which is intimately involved in the intiation and regulation of apoptosis. Moreover, PBR ligands have been shown to modulate MPTP and apoptotic response (see Carayon et al. (1995) Blood 87(8): p 3170; Hirsch et al (1998) Experimental Cell. Research 241 no 2:pp 426-434; and Bono et al (1999) Biochem. Biophys. Res. Comm. pp 265:457). Several recent reports have linked PBR and cancer based on alteration of PBR expression in tumor cells and PBR-dependent apoptotic modulations. Some of the highest densities of PBR are observed in neoplastic tissues and cell lines. Ovarian, hepatic and colon carcinomas, adenocarcinoma, glioma and breast cancer cells all show increased PBR densities relative to untranformed tissues (see Miettinen et al (1995) Cancer Res. (1995) 55, pp 2691-2695; Katz et al (1990) Clin. Sci. 78, pp 155; Katz et al (1990) Oncology 47, pp 139; and Venturini et al (1999) Life Sci. 65, pp 2223).

PBR ligands, both endogenous and synthetic, have been shown to have antiproliferative and pro-apoptotic properties. For example, the therapeutic potency of porphrins for the treatment of skin, bladder and lung cancers are reportedly linked to their affinities to PBR, and the sentitivity of tumor cell lines to photodynamic therapy reportedly parallel their PBR densities (Verma et al (1998) Mol. Med. 4(1), pp 40; Kupczyk-Subotkowska et al (1997) J. Med. Chem. 40(11), pp 1726; and Guo et al (2001) Cancer Chemother. Pharmacol. 48(2), pp 169). In addition to indirect PBR-based anticancer therapies, some PBR ligands have direct anticancer properties, including apoptotic and cell cycle inhibitor properties (Wang et al (1984) Proc. Natl. Acad. Sci. USA 81, pp 753; Landau et al (1998) J. Biochem. Pharmacol. 56, pp 1029; and Stoebner et al (2001) Cell Death Differ. 8(7), pp 747). Similarly, the dibenzodiazepinone analogues and derivatives of the present invention, in particular Compound 1, have been shown to bind to the PBR and inhibit cellular proliferation in a panel of different types of tumor cell lines, including low and high-grade gliomas. Compound 1 also increases expression of several genes involved in the regulation of apoptosis and signal transduction, as well as genes involved in steroid biosynthesis. Since human glioblastomas have an increased density of PBR compared with normal human brain, Compound 1's anticancer activity is believed to be via interaction with the PBR. Compound 1 has been shown to penetrate into brain tissues.

Alternatively, or in addition, the dibenzodiazepinone analogues and derivatives of the invention may have chemosensitizing or multidrug resistance modulating activity, as has been reported for other PBR ligands. For example, a non-cytotoxic dose of PK11195 increased the efficacy of a daunorubicin treatment on human multidrug-resistant leukemia cells in vitro and in vivo (Jakubikova et al (2002) Neoplasma 49(4), pp 231; and Decaudin et al (2002) Cancer Res. 62(5), pp 1388). Thus, the dibenzodiazepinone analogues, like other PBR ligands, may inhibit the expression or activity of multi-drug resistance (MDR)-associated protein (MDRP) or multi-drug resistance protein-1 (MDR1). “Multi-drug resistance” (MDR) broadly refers to a pattern of resistance to a variety of chemotherapeutic drugs with unrelated chemical structures and different mechanisms of action. Although the etiology of MDR is multifactorial, the overexpression of P-glycoprotein (Pgp), a membrane protein that mediates the transport of MDR drugs, remains the most common alteration underlying MDR in laboratory models (Childs, S 91994) Imp. Adv. Oncol. Pp 21-36). Moreover, expression of Pgp has been linked to the development of MDR in human cancer, particularly in the leukemias, lymphomas, multiple myeloma, neuroblastoma, and soft tissue sarcoma (Fan., D., et al (1993) Reversal of Multidrug Resistance in Cancer, ed. Kellen, J. A. (CRC, Boca Raton, Fla.), pp. 93-125). Recent studies showed that tumor cells expressing MDRP (Cole et al (1992) Science 258, pp 1650-1654) and lung resistance protein (LRP) (Scheffer et al (1995) Nat. Med. 1, pp 578-582) and mutation of DNA topoisomerase II (Beck (1989) J. Natl. Cancer Inst. 81, pp 1683-1685) also may render MDR.

While the above suggests that the dibenzodiazepinone analogues and derivatives of the invention may exert anticancer effects via interaction with the PBR, the mechanism of action may also be due, at least in part, to some as yet undefined mechanism or pathway. Alternatively or in addition to PBR, the dibenzodiazepinone analogues and derivatives of the present invention may bind to or interact with other cancer-associated proteins and polypeptides, including, without limitation, polypeptides encoded by oncogenes, polypeptides that induce angiogenesis, proteins involved in metastasizing and/or invasive processes, and proteases that regulate apoptosis and the cell cycle. Regardless of the mechanism of action, the dibenzodiazepinone analogues of the invention have been demonstrated to exhibit anti-cancer activity both in vitro and in vivo. Based on these discoveries, applicants have developed methods for treating neoplasms.

As used herein, the terms “neoplasm”, “neoplastic disorder”, “neoplasia” “cancer,” “tumor” and “proliferative disorder” refer to cells having the capacity for autonomous growth, i.e., an abnormal state of condition characterized by rapidly proliferating cell growth which generally forms a distinct mass that show partial or total lack of structural organization and functional coordination with normal tissue. The terms are meant to encompass hematopoietic neoplasms (e.g. lymphomas or leukemias) as well as solid neoplasms (e.g. sarcomas or carcinomas), including all types of pre-cancerous and cancerous growths, or oncogenic processes, metastatic tissues or malignantly transformed cells, tissues, or organs, irrespective of histopathologic type or stage of invasiveness. Hematopoietic neoplasms are malignant tumors affecting hematopoietic structures (structures pertaining to the formation of blood cells) and components of the immune system, including leukemias (related to leukocytes (white blood cells) and their precursors in the blood and bone marrow) arising from myeloid, lymphoid or erythroid lineages, and lymphomas (relates to lymphocytes). Solid neoplasms include sarcomas, which are malignant neoplasms that originate from connective tissues such as muscle, cartilage, blood vessels, fibrous tissue, fat or bone. Solid neoplasms also include carcinomas, which are malignant neoplasms arising from epithelial structures (including external epithelia (e.g., skin and linings of the gastrointestinal tract, lungs, and cervix), and internal epithelia that line various glands (e.g., breast, pancreas, thyroid). Examples of neoplasms that are particularly susceptible to treatment by the methods of the invention include leukemia, and hepatocellular cancers, sarcoma, vascular endothelial cancers, breast carcers, central nervous system cancers (e.g. astrocytoma, gliosarcoma, neuroblastoma, oligodendroglioma and glioblastoma), prostate cancers, lung and bronchus cancers, larynx cancers, esophagus cancers, colon cancers, colorectal cancers, gastrointestinal cancers, melanomas, ovarian and endometrial cancer, renal and bladder cancer, liver cancer, endocrine cancer (e.g. thyroid), and pancreatic cancer.

The dibenzodiazepinone analogue or derivative is brought into contact with or introduced into a cancerous cell or tissue. In general, the methods of the invention for delivering the compositions of the invention in vivo utilize art-recognized protocols for delivering therapeutic agents with the only substantial procedural modification being the substitution of the compound of the present invention for the therapeutic agent in the art-recognized protocols. The route by which the compound is administered, as well as the formulation, carrier or vehicle will depend on the location as well as the type of the neoplasm. A wide variety of administration routes can be employed. The compound may be administered by intravenous or intraperitoneal infusion or injection. For example, for a solid neoplasm that is accessible, the compound of the invention may be administered by injection directly into the neoplasm. For a hematopoietic neoplasm the compound may be administered intravenously or intravascularly. For neoplasms that are not easily accessible within the body, such as metastases or brain tumors, the compound may be administered in a manner such that it can be transported systemically through the body of the mammal and thereby reach the neoplasm and distant metastases for example intrathecally, intravenously or intramuscularly or orally. Alternatively, the compound can be administered directly to the tumor. The compound can also be administered subcutaneously, intraperitoneally, topically (for example for melanoma), rectally (for example colorectal neoplasm) vaginally (for example for cervical or vaginal neoplasm), nasally or by inhalation spray (for example for lung neoplasm).

The dibenzodiazepinone analogue or derivative is administered in an amount that is sufficient to inhibit the growth or proliferation of a neoplastic cell, or to treat a neoplastic disorder. The term “inhibition” refers to suppression, killing, stasis, or destruction of cancer cells. The inhibition of mammalian cancer cell growth according to this method can be monitored in several ways. Cancer cells grown in vitro can be treated with the compound and monitored for growth or death relative to the same cells cultured in the absence of the compound. A cessation of growth or a slowing of the growth rate (i.e., the doubling rate), e.g., by 50% or more at 100 micromolar, is indicative of cancer cell inhibition (see Anticancer Drug Development Guide: preclinical screening, clinical trials and approval; B. A. Teicher and P. A. Andrews, ed., 2004, Humana Press, Totowa, N.J.). Alternatively, cancer cell inhibition can be monitored by administering the compound to an animal model of the cancer of interest. Examples of experimental non-human animal cancer models are known in the art and described below and in the examples herein. A cessation of tumor growth (i.e., no further increase in size) or a reduction in tumor size (i.e., tumor volume by least a 58%) in animals treated with the compound relative to tumors in control animals not treated with the compound is indicative of significant tumor growth inhibition (see Anticancer Drug Development Guide: preclinical screening, clinical trials and approval; B. A. Teicher and P. A. Andrews, ed., 2004, Humana Press, Totowa, N.J.).

The term “treatment” refers to the application or administration of a dibenzodiazepinone analogue or derivative to a mammal, or application or administration of a dibenzodiazepinone analogue or derivative to an isolated tissue or cell line from a mammal, who has a neoplastic disorder, a symptom of a neoplastic disorder or a predisposition toward a neoplastic disorder, with the purpose to cure, heal, alleviate, relieve, alter, ameliorate, improve, or control the disorder, the symptoms of disorder, or the predisposition toward disorder. The term “treating” is defined as administering, to a mammal, an amount of a dibenzodiazepinone analogue or derivative sufficient to result in the prevention, reduction or elimination of neoplastic cells in a mammal (“therapeutically effective amount”). The therapeutically effective amount and timing of dosage will be determined on an individual basis and may be based, at least in part, on consideration of the age, body weight, sex, diet and general health of the recipient subject, on the nature and severity of the disease condition, and on previous treatments and other diseases present. Other factors also include the route and frequency of administration, the activity of the administered compound, the metabolic stability, length of action and excretion of the compound, drug combination, the tolerance of the recipient subject to the compound and the type of neoplasm or proliferative disorder. In one embodiment, a therapeutically effective amount of the compound is in the range of about 0.01 to about 750 mg/kg of body weight of the mammal. In another embodiment, the therapeutically effective amount is in the range of about 0.01 to about 300 mg/kg body weight per day. In yet another embodiment, the therapeutically effective amount is in the range of 10 to about 50 mg/kg body weight per day. The therapeutically effective doses of the above embodiments may also be expressed in milligrams per square meter (mg/m²) in the case of a human patient. Conversion factors for different mammalian species may be found in:Freireich et al, Quantitative comparison of toxicity of anticancer agents in mouse, rat, dog, monkey and man, Cancer Chemoth. Report, 1966, 50(4): 219-244. When special requirements may be needed (e.g. for children patients), the therapeutically effective doses described above may be outside the ranges stated herein. Such higher or lower doses are within the scope of the present invention.

To monitor the efficacy of tumor treatment in a human, tumor size and/or tumor morphology is measured before and after initiation of the treatment, and treatment is considered effective if either the tumor size ceases further growth, or if the tumor is reduced in size, e.g., by at least 10% or more (e.g., 20%, 30%, 40%, 50%, 60%, 70%, 80%, 90% or even 100%, that is, the absence of the tumor). Prolongation of survival, time-to-disease progression, partial response and objective response rate are surrogate measures of clinical activity of the investigational agent. Tumor shrinkage is considered to be one treatment-specific response. This system is limited by the requirement that patients have visceral masses that are amenable to accurate measurement. Methods of determining the size of a tumor in vivo vary with the type of tumor, and include, for example, various imaging techniques well known to those in the medical imaging or oncology fields (MRI, CAT, PET, etc.), as well as histological techniques and flow cytometry. For certain types of cancer, evaluation of serum tumor markers are also used to evaluate response (eg prostate-specific antigen (PSA) for prostate cancer, and carcino-embryonic antigen (CEA), for colon cancer). Other methods of monitoring cancer growth include cell counts (e.g. in leukemias) in blood or relief in bone pain (e.g. prostate cancer).

The dibenzodiazepinone compound may be administered in conjunction with or in addition to known other anticancer treatments such as radiotherapy, or other known anticancer compounds or chemotherapeutic agents. Such agents include, but are not limited to, 5-fluorouracil, mitomycin C, methotrexate, hydroxyurea, cyclophosphamide, dacarbazine, mitoxantrone, anthracyclines (Epirubicin and Doxurubicin), etopside, pregnasome, platinum compounds such as carboplatin and cisplatin, taxanes such as Paclitaxel™ and Docetaxel™; hormone therapies such as tamoxifen and anti-estrogens; antibodies to receptors, such as herceptin and Iressa; aromatase inhibitors, progestational agents and LHRH analogues; biological response modifiers such as IL2 and interferons; multidrug reversing agents such as the cyclosporin analogue PSC 833.

Toxicity and therapeutic efficacy of dibenzodiazepinone compounds can be determined by standard pharmaceutical procedures in cell cultures or experimental animals. Therapeutic efficacy is determined in animal models as described above and in the examples herein. Toxicity studies are done to determine the lethal dose for 10% of tested animals (LD10). Animals are treated at the maximum tolerated dose (MTD): the highest dose not producing mortality or greater than 20% body weight loss. The effective dose (ED) is related to the MTD in a given tumor model to determine the therapeutic index of the compound. A therapeutic index (MTD/ED) close to 1.0 has been found to be acceptable for some chemotherapeutic drugs, a preferred therapeutic index for classical chemotherapeutic drugs is 1.25 or higher.

The data obtained from cell culture assays and animal studies can be used in formulating a range of dosage for use in humans. The dosage of compositions of the invention will generally be within a range of circulating concentrations that include the MTD. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range of the compound. Such information can be used to more accurately determine useful doses in humans. Levels in plasma may be measured, for example, by HPLC.

Animal models to determine antitumor efficacy of a compound are generally carried out in mice. Either murine tumor cells are inoculated subcutaneously into the hind flank of mice from the same species (syngeneic models) or human tumor cells are inoculated subcutaneously into the hind flank of severe combined immune deficient (SCID) mice or other immune deficient mice (nude mice) (xenograft models).

Advances in mouse genetics have generated a number of mouse models for the study of various human diseases including cancer. The MMHCC (Mouse models of Human Cancer Consortium), sponsored by the National Cancer Institute, provides disease-site-specific compendium of known cancer models, and provides access to the searchable Cancer Models Database, as well as the NCI-MMHCC mouse repository. Mouse repositories can also be found at: The Jackson Laboratory, Charles River Laboratories, Taconic, Harlan, Mutant Mouse Regional Resource Centers (MMRRC) National Network and at the European Mouse Mutant Archive. Such models may be used for in vivo testing of dibenzodiazepinone compounds, as well as for determining a therapeutically effective dose.

In addition to the compounds of the invention, pharmaceutically acceptable salts, solvates or prodrugs of said compounds may also be employed in compositions to treat or prevent the above-identified disorders.

### VI. METHOD OF INHIBITING LIPOXYGENASE

In another embodiment, the present invention also provides for a method of treating diseased states, in particular inflammation, caused by the 5-lipoxygenase system and/or by the synthesis of the Leukotrienes C₄, D₄, E₄ and F₄ as well as Leukotriene B₄ in mammals, especially in human subjects. This method comprises administering to a subject an effective amount of a compound of Formula I. The compound of Formula I may be used alone or in combination with other anti-inflammatory compounds to treat or prevent disease states related to inflammation including pulmonary conditions, inflammation, cardiovascular conditions, central nervous system conditions or skin conditions. More specific diseases include gastritis; erosive esophagitis; inflammatory bowel disease; ethanol-induced hemorrhagic erosions; hepatic ischemia; ischemic neuronal injury; noxious agent induced damage or necrosis of hepatic, pancreatic, renal, neuronal or myocardial tissue; liver parenchymal damage caused by hepatoxic agents such as CCl₄ and D-galactosamine; ischemic renal failure; disease-induced hepatic damage; trauma- or stress-induced cell damage; asthma, multiple sclerosis, ischemic reperfusion, edema, rheumatoid arthritis, viral encephalitis, bacterial pneumonia, neurodegeneration, Alzheimer's disease and glycerol-induced renal failure.

For the method of the invention related to the 5-lipoxygenase system and/or the biosynthesis of leukotrienes, a typical effective unit dose of a compound of Formula I, or an analogue given orally or parenterally would be from about 5 to about 100 mg/kg of body weight of the subject with a daily dose ranging from about 15 to about 300 mg/kg of body weight of the subject.

The inhibition of lipoxygenase enzymes is monitored using methods well known in the art and as described in the examples herein. A decrease in enzyme activity by at least 10%, relative to the activity in the absence of a compound as described herein is indicative of effective inhibition of lipoxygenase activity.

The compounds useful according to the invention can be used to reduce or prevent inflammation. Among the hallmarks of local acute inflammation are heat, redness, swelling, pain and loss of function. These changes are induced largely by changes in vascular flow and caliber, changes in vascular permeability and leukocyte exudation (Robbins et al., “Pathologic Basis of Disease”, 6^{th} Ed., W.B. Saunders Co., Philadelphia, Pa.). Anti-inflammatory therapy performed using compounds useful according to the invention can be monitored for success by tracking any of these changes. For example, a decrease in swelling (e.g., at least 10% decrease following treatment) or reported pain (e.g., a sustained decrease of 1 point or more on a 1-10 scale reported by the patient, with 10 being the worst pain experienced in association with this disorder prior to treatment, and 0 being no pain) can be used to indicate successful treatment.

Other measurable hallmarks of inflammation include leukocyte infiltration and inflammatory cytokine levels. These hallmarks can be monitored by biopsy of the affected tissue. A decrease of 10% or more in leukocyte infiltration in fixed, stained tissue relative to infiltration in similar tissue prior to treatment can be used to indicate successful treatment, as can a decrease of 10% or more in the level of any given inflammatory cytokine, relative to the level before treatment. Those skilled in the art can readily assay for inflammatory cytokine levels in tissue, blood, or other fluid samples. Alternatively, the level of systemic indicators of inflammation such as C-reactive protein levels and erythrocyte sedimentation rate can be monitored. Each of these has established normal ranges in medicine, and treatment is considered successful if one or more of such indicators goes from outside the normal range to inside the normal range after the initiation of treatment.

In addition to the compounds of this invention, pharmaceutically acceptable salts, solvates or prodrugs of the compounds of this invention may also be employed in compositions to treat or prevent the above-identified disorders.

### VII. FURTHER ASPECTS OF THE INVENTION

The present invention includes a method for evaluating the chemotherapeutic potential of a candidate molecule, comprising: (a) determining the ability of a candidate molecule to inhibit the in vitro growth of a cancer cell; (b) determining the ability of the candidate molecule to bind a cellular receptor produced by a cancer cell, wherein said receptor is produced in a greater amount by the cancer cell than by a normal cell; and (c) determining the ability of the candidate molecule to inhibit the activity of at least one protein member of the MAPK pathway, thereby evaluating chemotherapeutic potential of a candidate molecule.

In one embodiment of this method, the candidate molecule is a non-conjugated compound, such as a small molecule, and includes a dibenzodiazepinone analogue or a dibenzodiazepinone analogue derivative of the present invention.

In a further embodiment of this method, the determining of (b) is determined by a competitive binding assay between the candidate molecule and a cognate ligand of said cellular receptor. The skilled artidan will understand that if the candidate molecule can successfully compete for binding to the receptor over the cognate ligand, such evidence will suggest that the candidate compound will specifically recognize and bind the receptor.

In an additional embodiment of this method, the receptor is a peripheral benzodiazepinone receptor (PBR).

In another embodiment of this method, the at least one protein member of the MAPK pathway is encoded for by a proto-oncogene, preferably a member of the Ras-MAPK signaling pathway family such as Ras, preferably K-Ras or Raf.

The present invention is also directed to a compound identified as having each of activities (a), (b) and (c), where activities (a), (b) and (c) are: (a) the ability to inhibit the in vitro growth of a cancer cell; (b) the ability to bind a cellular receptor produced by a cancer cell, wherein said receptor is produced in a greater amount by the cancer cell than by a normal cell; and (c) the ability to inhibit the activity of at least one protein member of the MAPK pathway.

The present invention is further directed to a method for lessening a non-specific cytotoxicity side-effect of an anti-cancer treatment administered to a mammal, comprising administrating to a mammal having cancer a molecule having each of activities (a), (b) and (c), wherein activities (a), (b) and (c) are: (a) the ability to inhibit the in vitro growth of a cancer cell; (b) the ability to bind a cellular receptor produced by a cancer cell, wherein said receptor is produced in a greater amount by the cancer cell than by a normal cell; and (c) the ability to inhibit the activity of at least one protein member of the MAPK pathway.

Similarly, the present invention is directed to a method for lessening a non-specific cytotoxicity side-effect of an anti-cancer treatment administered to a mammal, comprising administrating to a mammal having cancer a molecule having each of activities (a), (b) and (c), wherein said molecule is not Compound 1, and wherein activities (a), (b) and (c) are: (a) the ability to inhibit the in vitro growth of a cancer cell; (b) the ability to bind a cellular receptor produced by a cancer cell, wherein said receptor is produced in a greater amount by the cancer cell than by a normal cell; and (c) the ability to inhibit the activity of at least one protein member of the MAPK pathway.

The present invention also includes a method of evaluating a candidate molecule, comprising: (a) determining the ability of a candidate molecule to bind to a peripheral benzodiazepinone receptor (PBR) with an IC₅₀ (μM) of less than 5; and (b) determining the ability of the candidate molecule to reduce a phosphorylation activity in the MAPK pathway in a cell upon application of said candidate molecule to said cell in a concentration range of 3 μM to 30 μM, thereby evaluating a candidate molecule.

In one embodiment of this method, the candidate molecule is not Compound 1.

In another embodiment of this method, the evaluation is an evaluation of anti-cancer activity.

In a further embodiment of this method, the candidate molecule is a non-conjugated molecule, such as a small molecule.

In an additional embodiment of this method, the phosphorylation activity is reduced by inhibiting the activity of a Ras MAPK family member. In further embodiments, the Ras MAPK is Ras, K-Ras or Raf.

In yet another embodiment of this method, the candidate molecule is a compound of Formula 1. In a related embodiment, the candidate molecule is a compound of Formula 1 but not Compound 1. Preferred candidate molecules include Compound 97, Compound 99 and Compound 100.

The present invention is also directed to a compound having activities (a) and (b), where activities (a) and (b) are: (a) the ability to bind to a peripheral benzodiazepinone receptor (PBR) with an IC₅₀ (μM) of less than 5; and (b) the ability to reduce a phosphorylation activity in a MAPK pathway in a cell upon application of said candidate molecule to said cell in a concentration range of 3 μM to 30 μM.

The present invention is also directed to a method for lessening a non-specific cytotoxicity side-effect of an anti-cancer treatment administered to a mammal, comprising administrating to a mammal having cancer a molecule having each of activities (a) and (b), wherein activities (a) and (b) are: (a) the ability to bind to a peripheral benzodiazepinone receptor (PBR) with an IC₅₀ (μM) of less than 5; and (b) the ability to reduce a phosphorylation activity in a MAPK pathway in a cell upon application of said candidate molecule to said cell in a concentration range of 3 μM to 30 μM.

The present invention is further directed to a method for lessening a non-specific cytotoxicity side-effect of an anti-cancer treatment administered to a mammal, comprising administrating to a mammal having cancer a molecule having each of activities (a) and (b), wherein said molecule is not Compound 1, and wherein activities (a) and (b) are: (a) the ability to bind to a peripheral benzodiazepinone receptor (PBR) with an IC₅₀ (μM) of less than 5; and (b) the ability to reduce a phosphorylation activity in a MAPK pathway in a cell upon application of said candidate molecule to said cell in a concentration range of 3 μM to 30 μM.

The skilled artisan will understand that in the evaluative methods described above, the methods can be used to quickly screen a large number of molecules, and thereby identifying a small group of compounds with shared activities and with a high potential to be useful as a cancer treatment. The method can be used to screen a large number of compounds, including those having the same or differing structures and functions, as well as small groups of compounds, again having the same or differing structures and functions. In preferred embodiments, the method is used to screen a small group of compounds, sharing the same activity and a core structure. In such an embodiment, the method may be used to screen a group of at least 10, 20, 30, 40, 50 or more compounds.

The skilled artisan will also understand that additional methods can be used to confirm the chemotherapeutic activity of the molecules identified by the evaluative methods discussed above.

The present invention is also directed to a compound of the formula:

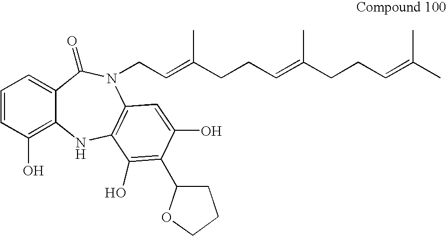

or a pharmaceutically acceptable salt thereof.

The present invention is also directed to a pharmaceutical composition comprising a compound of the formula:

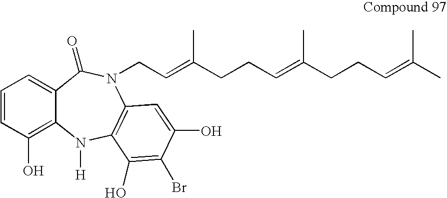

or a pharmaceutically acceptable salt thereof, together with a pharmaceutically acceptable carrier.

The present invention is also directed to a pharmaceutical composition comprising a compound of the formula:

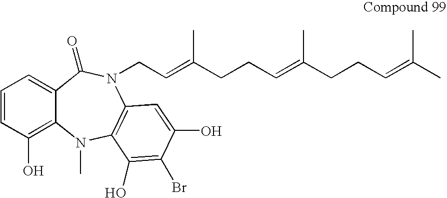

or a pharmaceutically acceptable salt thereof, together with a pharmaceutically acceptable carrier.

The present invention is additionally directed to a pharmaceutical composition comprising a compound of the formula:

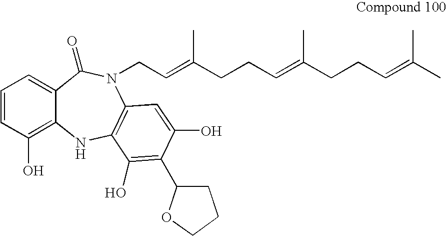

or a pharmaceutically acceptable salt thereof, together with a pharmaceutically acceptable carrier.

### EXAMPLES

Unless otherwise noted, all reagents were purchased from Sigma Chemical Co. (St. Louis, Mo.), Aldrich.

All NMR spectra were collected in deuterated solvent on a Varian 500™ Spectrometer (¹H NMR at 500 MHz, ¹³C NMR at 125 MHz). UV and mass spectra were collected by Waters 2690™ HPLC using a photodiode array detector (PDA, 210-400 nm) coupled to a Waters Micromass™ ZQ™ mass detector.

Unless otherwise indicated, all numbers expressing quantities of ingredients, properties such as molecular weight, reaction conditions, molar equivalents (eq), percentage of binding and/or inhibition, GI₅₀, IC₅₀ and so forth used in the specification and claims are to be understood as being modified in all instances by the term “about”. Accordingly, unless indicated to the contrary, the numerical parameters set forth in the present specification and attached claims are approximations. At the very least, and not as an attempt to limit the application of the doctrine of equivalents to the scope of the claims, each numerical parameter should at least be construed in light of the number of significant figures and by applying ordinary rounding techniques. Notwithstanding that the numerical ranges and parameters setting forth the broad scope of the invention are approximations, the numerical values set in the examples, Tables and Figures are reported as precisely as possible. Any numerical values may inherently contain certain errors resulting from variations in experiments, testing measurements, statistical analyses and such.

In the following section, examples describe in detail the chemical synthesis of representative compounds of the present invention. The procedures are illustrations, and the invention should not be construed as being limited by chemical reactions and conditions they express. No attempt has been made to optimize the yields obtained in these reactions, and it would be obvious to one skilled in the art that variations in reaction times, temperature, solvent and/or reagents could increase the yields.

In addition, the materials, methods, and examples, including in vitro and in vivo efficacy, bioavailability, toxicity and pharmacological properties are illustrative only and not intended to be limiting. Although methods and materials similar or equivalent to those described herein can be used in the practice or testing of the present invention, suitable methods and materials are described below. In case of conflict, the present specification, including definitions, will control.

### Example 1

### Production of Compound 1 by Fermentation

### a) Fermentation Procedure:

Micromonospora sp. (deposit accession number IDAC 070303-01) was maintained on agar plates of ISP2 agar (Difco Laboratories, Detroit, Mich.). An inoculum for the production phase was prepared by transferring the surface growth of the Micromonospora sp. from the agar plates to 125-mL flasks containing 25 mL of sterile medium comprised of glucose 10 g, potato dextrin type IV (Sigma) 20 g, yeast extract 5 g, N Z Amine-A 5 g, 1 g CaCO₃ made up to one liter with tap water (pH 7.0). The culture was incubated at about 28° C. for approximately 70-72 hours on a rotary shaker set at 250 rpm. Following incubation, 10 mL of culture was transferred to a 2 L baffled flask containing 600 mL of sterile production medium containing 20 g/L potato dextrin type IV (sigma), 30 g/L glycerol, 2.5 g/L Bacto-peptone, 8.34 g/L yeast extract, 3 g/L CaCO₃, pH 7.0. Fermentation broth was prepared by incubating the production culture at 28° C. in a rotary shaker set at 250 rpm for 5 days.

### b) Alternate Procedure:

The fermentation was accomplished as a 1×10 L batch in a 14.5 L fermentor (BioFlo 110™ Fermentor, New Brunswick Scientific, Edison, N.J., USA) using an improved procedure described in CA patent application 2,466,340, filed Jan. 21, 2004.

Micromonospora sp. (deposit accession number IDAC 070303-01) was maintained on agar plates of ISP2 agar (Difco Laboratories, Detroit, Mich.). An inoculum for the production phase was prepared by transferring the surface growth of the Micromonospora sp. from the agar plates to 2-L flasks containing 500 mL of sterile medium comprised of 10 g glucose, 20 g potato dextrin, 5 g yeast extract, 5 g NZ-Amine A, and 1 g CaCO₃ made up to one liter with tap water (pH 7.0). The culture was incubated at about 28° C. for approximately 70 hours on a rotary shaker set at 250 rpm. Following incubation, 300 mL of culture was transferred to a 14.5 L fermentor containing 10 L of sterile production medium. Each liter of production medium was composed of 20 g potato dextrin, 30 g glycerol, 2.5 g Bacto-peptone, 8.34 g yeast extract, 0.3 mL Silicone defoamer oil (Chem Service), 0.05 ml Proflo Oil™ (Traders protein) and 3 g CaCO₃ made to one liter with distilled water and adjusted to pH 7.0. The culture was incubated at 28° C., with dissolved oxygen (dO₂) controlled at 25% in a cascade loop with agitation varied between 320-600 RPM and aeration set at a fixed rate of 0.5 v/vim.

In addition to the above medium, other preferred media for the production of Compound 1 by fermentation are provided in Table 1 (QB, MA, KH, RM, JA, FA, CL).

### Example 2

### Isolation of Compound 1

Several isolation procedures were used to purify Compound 1, three different conditions are exemplified herein.

### a) Isolation Procedure 1:

500 mL ethyl acetate was added to 500 mL of fermentation broth prepared as described in Example 1 above. The mixture was agitated for 30 minutes on an orbital shaker at 200 rpm to create an emulsion. The phases were separated by centrifugation and decantation. Between 4 and 5 g of anhydrous MgSO₄ was added to the organic phase, which was then filtered and the solvents removed in vacuo.

An ethyl acetate extract from 2 L fermentation was mixed with HP-20 resin (100 mL; Mitsubishi Casei Corp., Tokyo, Japan) in water (300 mL). Ethyl acetate was removed in vacuo, the resin was filtered on a Buchner funnel and the filtrate was discarded. The adsorbed HP-20 resin was then washed successively with 2×125 mL of 50% acetonitrile in water, 2×125 mL of 75% acetonitrile in water and 2×125 mL of acetonitrile.

Fractions containing Compound 1 were evaporated to dryness and 100 mg was digested in the 5 mL of the upper phase of a mixture prepared from chloroform, cyclohexane, methanol, and water in the ratios, by volume, of 5:2:10:5. The sample was subjected to centrifugal partition chromatography using a High Speed Countercurrent Chromatography (HSCC) system (Kromaton Technologies, Angers, France) fitted with a 200 mL cartridge and prepacked with the upper phase of this two-phase system. The HSCC was run with the lower phase mobile and Compound 1 was eluted at approximately one-half column volume. Fractions were collected and Compound 1 was detected by TLC of aliquots of the fractions on commercial Kieselgel 60F₂₅₄ plates. Compound could be visualized by inspection of dried plates under UV light or by spraying the plates with a spray containing vanillin (0.75%) and concentrated sulfuric acid (1.5%, v/v) in ethanol and subsequently heating the plate. Fractions contained substantially pure Compound 1, although highly colored. A buff-colored sample could be obtained by chromatography on HPLC as follows.

6 mg of sample was dissolved in acetonitrile and injected onto a preparative HPLC column (Xterra™ ODS (10 μm), 19×150 mm, Waters Co., Milford, Mass.), with a 9 mL/min flow rate and UV peak detection at 300 nm. The column was eluted with acetonitrile/buffer (5 mM of NH₄HCO₃) according to the following gradient shown in Table 2.

TABLE 2
Preparative HPLC gradient

| Time (min) | Water (%) | Acetonitrile (%) |
|---|---|---|
| 0 | 50 | 50 |
| 10 | 0 | 100 |
| 20 | 0 | 100 |
| 25 | 50 | 50 |
| 30 | 50 | 50 |

Fractions containing Compound 1 were combined, concentrated and lyophilized to give a yield of 3.8 mg compound.

### b) Isolation Procedure 2:

Compound 1 was also isolated using the following alternative protocol. At the end of the incubation period, the fermentation broth from the baffled flasks of Example 1 was centrifuged and the supernatant decanted from the pellet containing the bacterial mycelia. 100 mL of 100% MeOH was added to the mycelial pellet and the sample was stirred for 10 minutes and centrifuged for 15 minutes. The methanolic supernatant was decanted and saved. 100 mL of acetone was then added to the mycelial pellet and stirred for 10 minutes then centrifuged for 15 minutes. The acetonic supernatant was decanted and combined with the methanolic supernatant. Finally, 100 mL of 20% MeOH/H₂O was added to the mycelial pellet, stirred for 10 minutes and centrifuged for 15 minutes. The supernatant was combined with the acetonic and methanolic supernatants.

The combined supernatant was added to 400 ml of HP-20 resin in 1000 mL of water and the organics were removed in vacuo. The resulting slurry was filtered on a Buchner funnel and the filtrate was discarded. HP-20 resin was washed successively with 2×500 mL of 50% MeOH/H₂O, 2×500 mL of 75% MeOH/H₂O and 2×500 mL of MeOH.

The individual washes were collected separately and analyzed by TLC as described above. Those fractions containing Compound 1 were evaporated to near dryness and lyophilized. The lyophilizate was dissolved in methanol and injected onto a preparative HPLC column (Xterra™ ODS (10 μm), 19×150 mm, Waters Co., Milford, Mass.) with a flow rate of 9 mL/min and peak detection at 300 nm.

The column was eluted with acetonitrile/buffer (5 mM of NH₄HCO₃) according to gradient shown in Table 3.

TABLE 3
Preparative HPLC gradient

| Time (min) | Buffer (%) | Acetonitrile (%) |
|---|---|---|
| 0 | 95 | 5 |
| 15 | 45 | 55 |
| 20 | 5 | 95 |
| 30 | 5 | 95 |
| 35 | 95 | 5 |

Fractions containing Compound 1 were combined, concentrated and lyophilized to yield about 33.7 mg of compound.

### c) Isolation Procedure 3:

10 liters of the whole broth from Example 1 was extracted twice with equal volumes of ethyl acetate and the two extracts were combined and concentrated to dryness. The dried extract was weighed, and for every gram of dry extract, 100 mL of MeOH—H₂O (2:1 v/v) and 100 mL of hexane was added. The mixture was swirled gently but well to achieve dissolution. The two layers were separated and the aqueous layer is washed with 100 mL of hexane. The two hexane layers were combined and the combined hexane solution was washed with 100 mL methanol:water (2:1, v/v). The two methanol:water layers were combined and treated with 200 mL of EtOAc and 400 mL of water. The layers were separated and the aqueous layer extracted twice further with 200 mL portions of EtOAc. The EtOAc layers are combined and concentrated. The residue obtained (220 mg) was suitable for final purification, either by HSCC or by HPLC as described above. This extraction process achieved a ten-fold purification when compared with the extraction protocol used in (a) or (b).

### Example 3

### Elucidation of the Structure of Compound 1

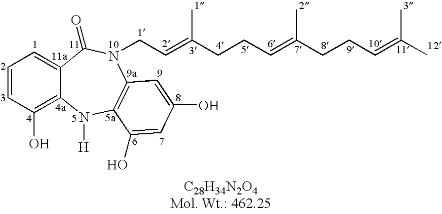

The calculated molecular weight of the major isotope (462.25) and formula (C₂₈H₃₄N₂O₄) of Compound 1 was confirmed by mass spectral analysis: negative ionization gave an (M−H)⁻ molecular ion of 461.2 and positive ionization gave an (M+H)⁺ molecular ion of 463.3. UVmax was determined to be 230 nm with a shoulder at 290 nm.

Proton and carbon NMR spectral analysis is shown in Table 4. NMR data were collected dissolved in MeOH-d₄ including proton, carbon and multidimensional pulse sequences gDQCOSY, gHSQC, gHMBC, and NOESY. A number of cross peaks in the 2D spectra of Compound 1 are key in the structural determination. For example, the farnesyl chain is placed on the amide nitrogen by a strong cross peak between the proton signal of the terminal methylene of that chain at 4.52 ppm and the amide carbonyl carbon at 170 ppm in the gHMBC experiment. This conclusion is confirmed by a cross peak in the NOESY spectrum between the same methylene signals at 4.52 ppm and the aromatic proton signal at 6.25 ppm from one of the two protons of the tetra substituted benzenoid ring. Assignment of proton and carbon signals are shown in Table 4.

TABLE 4
¹H and ¹³C NMR (δ_{H}, ppm) Data of Compound 1 in MeOH-D₄

| Assignment | ¹H | ¹³C | Group |
|---|---|---|---|
| 1 | 7.15 | 122.3 | CH |
| 2 | 6.74 | 121.0 | CH |
| 3 | 6.83 | 116.9 | CH |
| 4 | — | 146.0 | C—OH |
| 4a | — | 142.0 | C |
| 5a | — | 126.0 | C |
| 6 | — | 148.2 | C—OH |
| 7 | 6.20 | 100.0 | CH |
| 8 | — | 153.0 | C—OH |
| 9 | 6.25 | 101.0 | CH |
| 9a | — | 135.0 | C |
| 11 | — | 170.0 | C(O) |
| 11a | — | 125.0 | C |
| 1′ | 4.52 | 48.7 | CH₂ |
| 2′ | 5.35 | 121.1 | CH |
| 3′ | — | 138.5 | C |
| 4′ | 2.03 | 39.5 | CH₂ |
| 5′ | 2.08 | 26.7 | CH₂ |
| 6′ | 5.09 | 124.1 | CH |
| 7′ | — | 135.0 | C |
| 8′ | 1.95 | 39.6 | CH₂ |
| 9′ | 2.02 | 26.3 | CH₂ |
| 10′ | 5.06 | 124.4 | CH |
| 11′ | — | 130.9 | C |
| 12′ | 1.64 | 24.8 | CH₃ |
| 1″ | 1.72 | 15.5 | CH₃ |
| 2″ | 1.59 | 14.9 | CH₃ |
| 3″ | 1.55 | 16.5 | CH₃ |

Based on the mass, UV and NMR spectroscopy data, the structure of the compound was determined to be the structure of Compound 1 shown above.

### Example 4

### Dialkylsulfate Reactions

### a) Synthesis and Structural Elucidation of Compounds 2 and 80

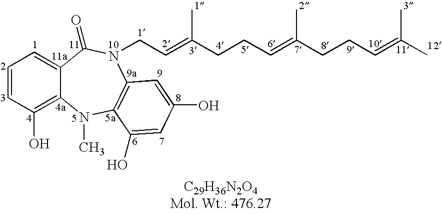

Compound 2, namely 10-farnesyl-4,6,8-trihydroxy-5-methyl-5,10-dihydro-dibenzo[b,e][1,4]diazepin-11-one, and

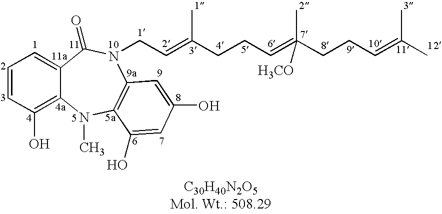

Compound 80, namely 10-(7-methoxy-3,7,11-trimethyldodeca-2,10-dienyl)-4,6,8-trihydroxy-5-methyl-5,10-dihydro-dibenzo[b,e][1,4]diazepin-11-one, were prepared and identified as follows:

i) Preparation:

Compound 1 (500.0 mg) was dissolved in methanol (MeOH, 20 mL) and stirred with dimethyl sulfate (0.5 mL) and NaHCO₃ (250 mg) at room temperature for 48 hrs. The reaction mixture was diluted to 200 mL by adding water and extracted with ethyl acetate (EtOAc, 300 mL×3). The organic layer was separated and dried under vacuum, re-dissolved in MeOH and filtered through a 0.45 pm 13 mm Acrodisc™ GHP syringe filter. The filtrate was subjected for isolation on a Waters HPLC coupled to a photodiode array detector. Compound 80 (12.1 mg) and Compound 2 (308.5 mg) were isolated by the multiple injections on Nova-Pack™ HR 6 μm C18 25×200 mm column (20 mL/min, H₂O/CH₃CN gradient 80:20-30:70, 0-8 min; 30:70-0:100, 8-18 min), eluting at 14.5 and 16.8 min, respectively.

ii) Structural Elucidation of Compounds 2 and 80:

The calculated molecular weight for the major isotope (476.27) and formula (C₂₉H₃₆N₂O₄) of Compound 2 was confirmed by mass spectral analysis: negative ionization gave an (M−H)⁻ molecular ion of 475.6 and positive ionization gave an (M+Na)⁺ molecular ion of 499.4. Proton and carbon NMR spectral analysis is shown in Table 5. Signals were easily assigned based on Compound 1 structure knowledge. The calculated molecular weight for the major isotope (508.29) and formula (C₃₀H₄₀N₂O₅) of Compound 80 was confirmed by mass spectral analysis: negative ionization gave an (M−H)⁻ molecular ion of 507.3 and positive ionization gave an (M+H)⁺ molecular ion of 509.3. The characteristic N-methyl (signal 5), methoxy (signal 7′-OMe) and the methylene group (6′), from the addition of methanol on the farnesyl chain were easily assigned as shown in Table 5.

TABLE 5
NMR (δ, ppm) Data of Compounds 2 and 80 in MeOH-D₄

| | Compound 2 | | Compound 80 | |
|---|---|---|---|---|
| Assignment | ¹H | ¹³C | ¹H | Group |
| 1 | 7.21 | 122.1 | 7.21 | CH |
| 2 | 7.14 | 127.3 | 7.14 | CH |
| 3 | 7.02 | 118.4 | 7.02 | CH |
| 4 | — | 152.6 | — | C—OH |
| 4a | — | 139.3 | — | C |
| 5-N-Me | 2.92 | 41.1 | 2.93 | N—CH₃ |
| 5a | — | 125.4 | — | C |
| 6 | — | 154.8 | — | C—OH |
| 7 | 6.22 | 99.6 | 6.20 | CH |
| 8 | — | 156.8 | — | C—OH |
| 9 | 6.34 | 101.4 | 6.34 | CH |
| 9a | — | 142.0 | — | C |
| 11 | — | 168.2 | — | C(O) |
| 11a | — | 133.5 | — | C |
| 1′ | 4.83, 4.58 | 47.7 | 4.89, 4.57 | CH₂ |
| 2′ | 5.44 | 119.8 | 5.42 | CH |
| 3′ | — | 139.3 | — | C |
| 4′ | 2.07^{b} | 39.5 | 2.06 | CH₂ |
| 5′ | 2.12^{b} | 26.2 | 1.42 | CH₂ |
| 6′ | 5.10 | 123.8 | 1.42 | CH(CH₂)^{a} |
| 7′ | — | 135.1 | — | C |
| 7′ OMe | N/A | N/A | 3.13 | OCH₃ |
| 8′ | 1.95^{b} | 39.8 | 1.42 | CH₂ |
| 9′ | 2.04^{b} | 26.8 | 1.93 | CH₂ |
| 10′ | 5.07 | 124.3 | 5.12 | CH |
| 11′ | — | 130.8 | — | C |
| 12′ | 1.65 | 24.9 | 1.68 | CH₃ |
| 1″ | 1.78 | 15.8 | 1.77 | CH₃ |
| 2″ | 1.60 | 15.1 | 1.10 | CH₃ |
| 3″ | 1.55 | 16.6 | 1.60 | CH₃ |

N/A: not applicable, group not present in the molecule
^{a}CH in Compound 2, CH₂ in Compound 80
^{b}Signals for 4′, 5′, 8′ and 9′ are very close; assignment was based on Compound 1

### b) Synthesis and Structural Elucidation of Compounds 14, 82, 83, 84, 85, and 87

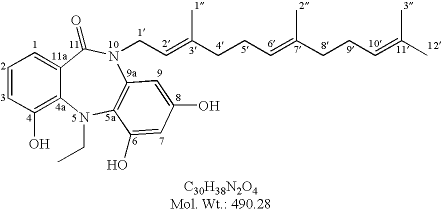

Compound 14: 10-farnesyl-4,6,8-trihydroxy-5-ethyl-5,10-dihydro-dibenzo[b,e][1,4]diazepin-11-one;

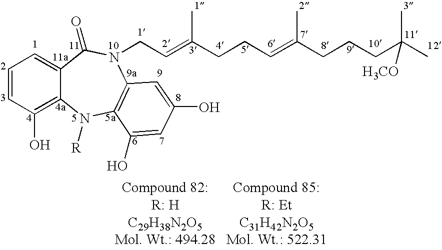

Compound 82: 10-(11-methoxy-3,7,11-trimethyl-2,6-dodecadienyl)-4,6,8-trihydroxy-5,10-dihydro-dibenzo[b,e][1,4]diazepin-11-one;

Compound 85: 10-(11-methoxy-3,7,11-trimethyl-2,6-dodecadienyl)-4,6,8-trihydroxy-5-ethyl-5,10-dihydro-dibenzo[b,e][1,4]diazepin-11-one;

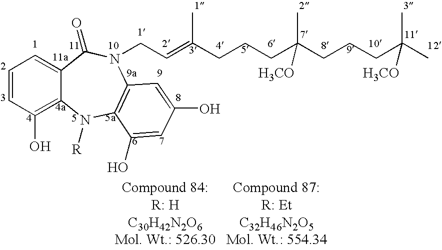

Compound 84: 10-(7,11-dimethoxy-3,7,11-trimethyl-2-dodecenyl)-4,6,8-trihydroxy-5,10-dihydro-dibenzo[b,e][1,4]diazepin-11-one;

Compound 87: 10-(7,11-dimethoxy-3,7,11-trimethyl-2-dodecenyl)-4,6,8-trihydroxy-5-ethyl-5,10-dihydro-dibenzo[b,e][1,4]diazepin-11-one;

i) Preparation:

Compound 1 (85.3 mg) was stirred for 72 hrs at room temperature in a mixture of diethyl sulfate (2.0 mL) and NaHCO₃ (99.9 mg) in MeOH (2 mL). The resulting mixture was filtered through a 0.45 μm 13 mm Acrodisc™ GHP syringe filter. The solution was purified by preparative HPLC (multiple injections on a NovaPack™ C-18 25×200 mm column (20 mL/min, H₂O/CH₃CN gradient 80:20-30:70, 0-8 min; 30:70-0:100, 8-18 min) to give four major peaks: Compound 14 (20.0 mg with some impurities, RT: 16.6 min), Compound 82 (5.65 mg, RT: 11.6 min), Compound 84 (2.20 mg, RT: 10.3 min), Compound 85 (17.54 mg, RT: 14.3 min) and Compound 86 (7.82 mg, RT: 12.6 min) were obtained. The fraction containing Compound 14 was further purified by HPLC using the same column (20 mL/min, H₂O/CH₃CN gradient 80:20-30:70, 0-8 min; 30:70-0:100, 8-18 min, curve 7), to give substantially pure Compound 14 (13.85 mg, RT: 17.9 min).

ii) Structural Elucidation of Compounds 14, 82, 84, 85 and 87:

The calculated molecular weight of the major isotope (490.28) and formula (C₃₀H₃₈N₂O₄) of Compound 14 was confirmed by mass spectral analysis: negative ionization gave an (M−H)⁻ molecular ion of 489.3 and positive ionization gave an (M+H)⁺ molecular ion of 491.3. Proton NMR signals were easily assigned based on Compounds 1 and 2 structures knowledge. The characteristic N-ethyl group (5-N-Et) was easily assigned as shown in Table 6.

The calculated molecular weight of the major isotope (494.28) and formula (C₂₉H₃₈N₂O₅) of Compound 82 was confirmed by mass spectral analysis: negative ionization gave an (M−H)⁻ molecular ion of 493.3 and positive ionization gave an (M+H)⁺ molecular ion of 495.4, and a fragment having an (M+H−HOCH₃)⁺ molecular ion of 463.3. Proton NMR signals were easily assigned based on Compound 1 structure knowledge. The characteristic methoxy group (11′-OMe) and the methylene group (10′), from the addition of methanol on the farnesyl chain were easily assigned as shown in Table 6.

The calculated molecular weight (526.30) and formula (C₃₀H₄₂N₂O₆) of Compound 84 was confirmed by mass spectral analysis: negative ionization gave an (M−H)⁻ molecular ion of 525.3 and positive ionization gave an (M+H)+molecular ion of 527.4, and fragments having respectively (M+H−HOCH₃)⁺ and (M+H−HOCH₃—HOCH₃)⁺ molecular ion of 495.4 and 463.4. Proton NMR signals were easily assigned based on Compound 1 structure knowledge. The characteristic methoxy groups (signals 7′-OMe and 11′-OMe) from the addition of two molecules of methanol on the farnesyl chain were easily assigned as shown in Table 6. The methylene groups (5′, 6′, 8′, 9′ and 10′) were found to have similar chemical shifts, which is consistent with a saturated chain.

The calculated molecular weight of the major isotope (522.31) and formula (C₃₁H₄₂N₂O₅) of Compound 85 was confirmed by mass spectral analysis: negative ionization gave an (M−H)⁻ molecular ion of 521.3 and positive ionization gave an (M+H)⁺ molecular ion of 523.5, and a fragment having an (M+H−HOCH₃)⁺ molecular ion of 491.3. The characteristic N-ethyl group (5-N-Et), and the methoxy (11′-OMe) and methylene (10′) groups from the addition of methanol on the farnesyl chain were easily assigned as shown in Table 6.

The calculated molecular weight of the major isotope (554.34) and formula (C₃₂H₄₆N₂O₆) of Compound 87 was confirmed by mass spectral analysis: negative ionization gave an (M−H)⁻ molecular ion of 553.4 and positive ionization gave an (M+H)⁺ molecular ion of 555.4, and fragments having respectively (M+H−HOCH₃)⁺ and (M+H−HOCH₃—HOCH₃)⁺ molecular ion of 523.5 and 491.3. The characteristic N-ethyl group (5-N-Et), and methoxy (7′-OMe and 11′-OMe) groups from the addition of two molecules of methanol on the farnesyl chain were easily assigned as shown in Table 6. The methylene groups (5′, 6′, 8′, 9′ and 10′) were all found to have similar chemical shifts, which is consistent with the saturated alkyl group.

TABLE 6
¹H NMR (δ_{H}, ppm) Data of Compounds 14, 82, 84, 85 and 87
in MeOH-D₄

| Assignmen | 14 | 82 | 84 | 85 | 87 | Group |
|---|---|---|---|---|---|---|
| 1 | 7.20 | 7.17 | 7.17 | 7.20 | 7.20 | CH |
| 2 | 7.13 | 6.77 | 6.76 | 7.14 | 7.14 | CH |
| 3 | 7.02 | 6.85 | 6.83 | 7.03 | 7.03 | CH |
| 5-N-Et (C1) | 3.23, 3.15 | N/A | N/A | 3.23, 3.15 | 3.24, | CH₂ |
| | | | | | 3.16 | |
| 5-N-Et (C2) | 1.07 | N/A | N/A | 1.09 | 1.09 | CH₃ |
| 7 | 6.22 | 6.23 | 6.22 | 6.22 | 6.22 | CH |
| 9 | 6.34 | 6.28 | 6.27 | 6.34 | 6.34 | CH |
| 1′ | 4.58, 4.56 | 4.55 | 4.57 | 4.82, 4.56 | 4.91, | CH₂ |
| | | | | | 4.54 | |
| 2′ | 5.41 | 5.37 | 5.37 | 5.41 | 5.38 | CH |
| 4′ | 2.06 | 2.07 | 2.04 | 2.07 | 2.05 | CH₂ |
| 5′ | 2.11 | 2.13 | * | 2.13 | ** | CH₂ |
| 6′ | 5.10 | 5.12 | * | 5.11 | ** | CH(CH₂)^{a} |
| 7′-OMe | N/A | N/A | 3.18 | N/A | 3.17 | OCH₃ |
| 8′ | 1.95 | 1.97 | * | 1.95 | ** | CH₂ |
| 9′ | 2.04 | 1.40 | * | 1.38 | ** | CH₂ |
| 10′ | 5.07 | 1.40 | * | 1.38 | ** | CH(CH₂)^{b} |
| 11′-OMe | N/A | 3.14 | 3.11 | 3.13 | 3.12 | OCH₃ |
| 12′ | 1.65 | 1.10 | 1.16 | 1.08 | 1.14 | CH₃ |
| 1″ | 1.77 | 1.74 | 1.72 | 1.78 | 1.78 | CH₃ |
| 2″ | 1.60 | 1.61 | 1.10 | 1.10 | 1.10 | CH₃ |
| 3″ | 1.55 | 1.10 | 1.16 | 1.08 | 1.14 | CH₃ |

N/A: not applicable, group not present in the molecule
* signal 1.22-1.51 ppm, 10 protons
** signal 1.22-1.49 ppm, 10 protons
^{a}CH in Compounds 14, 82 and 85, CH₂ in Compounds 84 and 87
^{b}CH in Compound 14, CH₂ in Compounds 82, 84, 85 and 87

iii) Alternate Procedure, Preparation of Compounds 82 and 83:

Compound 1 (107.2 mg) and p-toluenesulfonic acid (PTSA, 13.8 mg) were stirred reflux in methanol for 30 minutes. The reaction was filtered and subjected to Waters HPLC purification (multiple injections on a NovaPack™ C-18 25×200 mm column: 20 mL/min, H₂O/CH₃CN gradient 70:30-20:80, 0-4 min; 20:80-0:100, 4-9 min) to give Compound 82 (8.5 mg, RT 7.2 min) and Compound 83 (4.3 mg, RT 7.7 min).

Structural elucidation of Compound 83 was done as for Compounds 82 and 84. Mass spectral analysis (ES⁺: 495.5; ES⁻: 493.3) confirmed a calculated molecular weight of the major isotope (494.28) and formula (C₂₉H₃₈N₂O₅) as for Compound 82. Proton NMR analysis showed signals 6′ (1.42 ppm, CH₂) and 7′-OMe (3.13 ppm, OCH₃) corresponding to the addition of a methanol molecule on the second double bond of the farnesyl group (as for Compound 80).

### c) Synthesis and Structural Elucidation of Compound 63

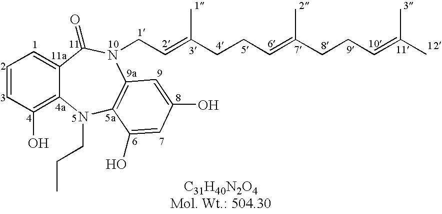

Compound 63, namely 10-farnesyl-4,6,8-trihydroxy-5-n-propyl-5,10-dihydro-dibenzo[b,e][1,4]diazepin-11-one, was prepared and identified as follows:

i) Preparation:

Compound 1 (46.7 mg) was stirred for 72 hrs at room temperature in a mixture of dipropyl sulfate (0.5 mL) and NaHCO₃ (46.3 mg) in MeOH (3 mL). The resulting mixture was filtered through a 0.45 μm 13 mm Acrodisc™ GHP syringe filter. The solution was purified by preparative HPLC (multiple injections on a NovaPack™ C-18 25×200 mm column: 20 mL/min, H₂O/CH₃CN gradient 80:20-30:70, 0-8 min; 30:70-0:100, 8-18 min) to give substantially pure Compound 63 (18.0 mg, RT: 17.3 min).

ii) Structural Elucidation of Compound 63:

The calculated molecular weight of the major isotope (504.30) and formula (C₃₁H₄₀N₂O₄) of Compound 63 was confirmed by mass spectral analysis: negative ionization gave an (M−H)⁻ molecular ion of 503.4 and positive ionization gave an (M+H)+molecular ion of 505.5. Proton NMR signals were easily assigned based on Compounds 1 and 2 structures knowledge. The characteristic N-Propyl group (5-N-Pr (C1 to C3)) was easily assigned as shown in Table 7 below.

### d) Synthesis and Elucidation of Compound 98

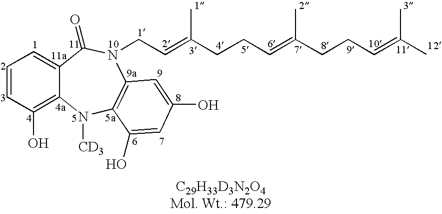

Compound 98: 10-Farnesyl-4,6,8-trihydroxy-5-(trideuteriomethyl)-5,10-dihydro-dibenzo[b,e][1,4]diazepin-11-one, was prepared and identified according to the following procedure:

i) Preparation:

Compound 1 (121.3 mg) was dissolved in MeOH (3.0 mL), dimethyl sulfate-d₆ (150 μL, CDN isotopes Inc.) and NaHCO₃ (58.1 mg) were added, and the reaction was stirred at room temperature overnight. The reaction mixture was filtered and the filtrate was subjected to Waters HPLC purification (multiple injections on Nova Pack™ C-18 25×200 mm column: 20 mL/min, H₂O/CH₃CN gradient 70:30-20:80, 0-4 min; 20:80-0:100, 4-9 min) to give Compound 98 (82.7 mg, RT 9.4 min).

ii) Structural Elucidation:

The calculated molecular weight of the major isotope (479.29) and formula (C₂₉H₃₃D₃N₂O₄) of Compound 98 was confirmed by mass spectral analysis: negative ionization gave an (M−H)⁻ molecular ion of 478.5, and positive ionization gave an (M+H)⁺ molecular ion of 480.6. The structure was further confirmed by proton NMR spectrum as shown in Table 7 below.

TABLE 7
¹H NMR (δ_{H}, ppm) Data of Compounds 63 and 98
in MeOH-D₄

| Assign | Compound 63 | Compound 98 | Group |
|---|---|---|---|
| 1 | 7.19 | 7.21 | CH |
| 2 | 7.12 | 7.14 | CH |
| 3 | 7.01 | 7.02 | CH |
| 5-N—Pr (C1) | 3.04, 3.15 | — | CH₂^{a} |
| 5-N—Pr (C2) | 1.50 | N/A | CH₂ |
| 5-N—Pr (C3) | 0.91 | N/A | CH₃ |
| 7 | 6.22 | 6.22 | CH |
| 9 | 6.34 | 6.34 | CH |
| 1′ | 4.54, 4.88 | 4.83, 4.59 | CH₂ |
| 2′ | 5.40 | 5.44 | CH |
| 4′ | 2.07 | 2.07 | CH₂ |
| 5′ | 2.12 | 2.12 | CH₂ |
| 6′ | 5.09 | 5.10 | CH |
| 8′ | 1.96 | 1.95 | CH₂ |
| 9′ | 2.03 | 2.03 | CH₂ |
| 10′ | 5.07 | 5.07 | CH |
| 12′ | 1.65 | 1.65 | CH₃ |
| 1″ | 1.78 | 1.77 | CH₃ |
| 2″ | 1.60 | 1.60 | CH₃ |
| 3″ | 1.55 | 1.55 | CH₃ |

N/A: not applicable, group not present in the molecule
^{a}CH₂ in Compound 63, CD₃ in Compound 98.

### Example 5

### Alkyl Halide Reactions

### a) Synthesis and Structural Elucidation of Compound 3

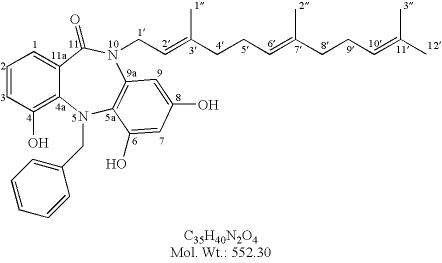

Compound 3, namely 5-benzyl-10-farnesyl-4,6,8-trihydroxy-5,10-dihydro-dibenzo[b,e][1,4]diazepin-11-one was prepared and identified as follows:

i) Preparation:

Compound 1 (60.5 mg) was stirred 84 hrs with benzyl chloride (1.8 mL, 120 eq, Sigma) in presence of two drops of pyridine (Aldrich). The resulting mixture was directly subjected to HPLC separation. Purification by multiple injection on a Waters™ RCM Nova-Pak™ HR C18 6 μm 60A 25×200 mm column (20 mL/min H₂O/CH₃CN 80:20-30:70, 0-8 min; 30:70-0:100, 8-18 min) gave Compound 3 (46.0 mg) with retention time of 17.5 min.

ii) Structural Elucidation:

The calculated molecular weight of the major isotope (552.30) and formula (C₃₅H₄₀N₂O₄) of Compound 3 was confirmed by mass spectral analysis: negative ionization gave an (M−H)⁻ molecular ion of 551.7 and positive ionization gave an (M+Na)⁺ molecular ion of 575.5. Proton NMR signals were easily assigned based on Compound 1 structure knowledge. The characteristic N-benzyl group (5-N-alkyl (C1-C5)) were assigned as shown in Table 8 below.

### b) Synthesis and Structural Elucidation of Compound 64

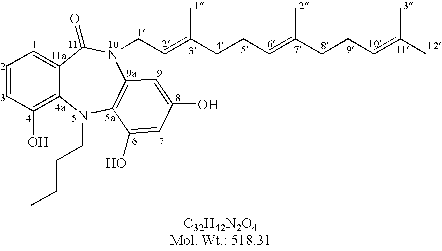

Compound 64, namely 10-farnesyl-4,6,8-trihydroxy-5-n-butyl-5,10-dihydro-dibenzo[b,e][1,4]diazepin-11-one, was prepared and identified as follows:

i) Preparation:

Compound 1 (43.5 mg) was stirred in 1-bromobutane (2.0 mL) with pyridine (50 μL) at 80° C. overnight. The reaction mixture was diluted with MeOH (1.0 mL), filtered and subjected for Waters HPLC as described above (in a) to give a semi-purified Compound 64 (RT: 18.1 min). The semi-purified compound was further purified using the same conditions (except with curve 7) to give substantially pure Compound 64 (10.5 mg, RT: 17.9 min).

ii) Structure Elucidation:

The calculated molecular weight of the major isotope (518.31) and formula (C₃₂H₄₂N₂O₄) of Compound 64 was confirmed by mass spectral analysis: negative ionization gave an (M−H)⁻ molecular ion of 517.4 and positive ionization gave an (M+H)⁺ molecular ion of 519.5. The characteristic N-n-butyl group (5-N-alkyl (C1-C4)) was easily assigned as shown in Table 8 below.

### c) Synthesis and Structural Elucidation of Compound 67

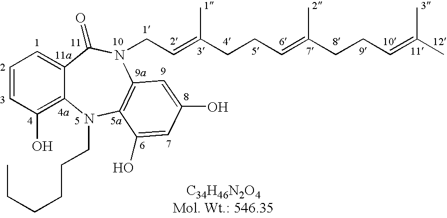

Compound 67, namely 10-farnesyl-4,6,8-trihydroxy-5-n-hexyl-5,10-dihydro-dibenzo[b,e][1,4]diazepin-11-one, was prepared and identified as follows:

i) Preparation:

Compound 1 (39.2 mg) was stirred in 1-bromohexane (2.0 mL) with pyridine (50 μL) at 80° C. overnight. The reaction mixture was diluted with MeOH (1.0 mL), filtered and subjected for Waters HPLC (multiple injections on a NovaPack™ C-18 25×200 mm column: 20 mL/min, H₂O/CH₃CN gradient 80:20-30:70, 0-8 min; 30:70-0:100, 8-18 min; isocratic CH₃CN 18-24 minutes) to give substantially pure Compound 67 (14.0 mg, RT: 20.1 min).

ii) Structural Elucidation:

The calculated molecular weight (546.35) and formula (C₃₄H₄₆N₂O₄) of Compound 67 was confirmed by mass spectral analysis: negative ionization gave an (M−H)⁻ molecular ion of 545.6 and positive ionization gave an (M+H)+molecular ion of 547.6. Proton NMR signals were easily assigned based on Compounds 1 and 2 structures knowledge. The characteristic N-n-hexyl group (5-N-alkyl (C₁-C₆)) was easily assigned as shown in Table 8 below.

### d) Synthesis and Structural Elucidation of Compound 77

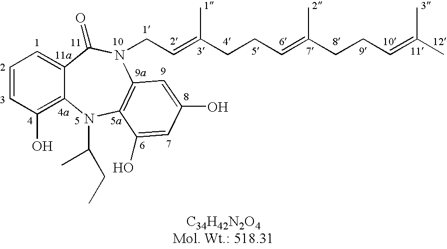

Compound 77, namely 10-Farnesyl-4,6,8-trihydroxy-5-sec-butyl-5,10-dihydro-dibenzo[b,e][1,4]diazepin-11-one, was prepared and identified as follows:

i) Preparation Procedure 1:

Compound 1 (26.0 mg) was stirred in 2-bromobutane (4.0 mL) with pyridine (100 μL) under reflux for one hour. The reaction mixture was concentrated in vacuo, diluted with MeOH (2.0 mL), filtered and subjected for Waters HPLC as described above (in a) to give Compound 77 (1.65 mg, RT: 18.0 min).

ii) Preparation Procedure 2:

Compound 1 (104.5 mg) was stirred in 2-bromobutane (5.0 mL) with pyridine (400 μL) under reflux for two hours. The reaction mixture was concentrated in vacuo, diluted with MeOH (4.0 mL), filtered and subjected for Waters HPLC (multiple injections on a NovaPack™ C-18 25×200 mm column: 20 mL/min, H₂O/CH₃CN gradient 70:30-20:80, 0-4 min; 20:80-0:100, 4-9 min) to give Compound 77 (7.38 mg, RT: 11.2 min).

iii) Structure Elucidation:

The calculated molecular weight of the major isotope (518.31) and formula (C₃₂H₄₂N₂O₄) of Compound 64 was confirmed by mass spectral analysis: negative ionization gave an (M−H)⁻ molecular ion of 517.4 and positive ionization gave an (M+H)+molecular ion of 519.6. The characteristic N-sec-butyl group (5-N-Alkyl (C1-C4)) was easily assigned as shown in Table 8 below.

TABLE 8
¹H NMR (δ_{H}, ppm) Data of Compounds 3, 64, 67 and 77 in
MeOH-D₄

| | | Compound | Compound | Compound | Compound | |
|---|---|---|---|---|---|---|
| Assign | | 3 | 64 | 67 | 77 | Group |
| 1 | | 7.19 | 7.20 | 7.20 | 7.19 | CH |
| 2 | | 7.09 | 7.13 | 7.12 | 7.14 | CH |
| 3 | | 6.96 | 7.02 | 7.02 | 7.01 | CH |
| 5-N-alkyl | (C1) | 4.34, 4.25 | 3.18, 3.09 | 3.18, 3.08 | 3.29 | ^{a} |
| | (C2) | — | 1.46 | 1.48 | 1.34 | ^{b} |
| | (C3) | 7.20 | 1.35 | 1.30 | 086, 0.81^{h} | ^{c} |
| | (C4) | 7.23 | 0.89 | 1.30 | 1.00, 0.96^{h} | ^{d} |
| | (C5)^{e} | 7.23 | N/A | 1.30 | N/A | ^{e} |
| | (C6) | N/A | N/A | 0.89 | N/A | ^{f} |

| | | | | | |
|---|---|---|---|---|---|
| 7 | 6.16 | 6.22 | 6.23 | 6.22 | CH |
| 9 | 6.31 | 6.34 | 6.33 | 6.34, 6.32^{h} | CH |
| 1′ | 4.55, 4.48 | 4.89, 4.54 | 4.92, 4.52 | 4.90, 4.53 | CH₂ |
| 2′ | 5.44 | 5.40 | 5.39 | 5.41, 5.36^{h} | CH |
| 4′^{g} | 2.08 | 2.06 | 2.06 | 2.06 | CH₂ |
| 5′^{g} | 2.13 | 2.12 | 2.11 | 2.13 | CH₂ |
| 6′ | 5.10 | 5.09 | 5.09 | 5.10 | CH |
| 8′^{g} | 1.94 | 1.96 | 1.96 | 1.97 | CH₂ |
| 9′^{g} | 2.01 | 2.04 | 2.04 | 2.05 | CH₂ |
| 10′ | 5.04 | 5.07 | 5.07 | 5.08 | CH |
| 12′ | 1.64 | 1.65 | 1.64 | 1.66 | CH₃ |
| 1″ | 1.79 | 1.78 | 1.78 | 1.80, 1.79^{h} | CH₃ |
| 2″ | 1.56 | 1.60 | 1.60 | 1.61 | CH₃ |
| 3″ | 1.52 | 1.55 | 1.56 | 1.56 | CH₃ |

N/A: not applicable, group not present in the molecule
^{a}CH₂ in Compounds 3, 64 and 67, and CH in Compound 77.
^{b}C in Compound 3, CH₂ in Compounds 64, 67 and 77.
^{c}CH(2H) in Compound 3, CH₂ in Compounds 64 and 67, and CH₃ in Compound 77.
^{d}CH(2H) in Compound 3, CH₃ in Compounds 64 and 77, and CH₂ in Compound 67.
^{e}CH in Compound 3, CH₂ in Compound 67, absent in Compounds 64 and 77.
^{f}Absent in Compounds 3, 64 and 77.
^{g}Signals at 4′, 5′, 8′ and 9′ are very close; assignment was based on Compound 1
^{h}From two different isomers.

Other N-Alkyl Compounds 60 to 62, 65, 66, and 68 to 76 are also prepared via this procedure, by reaction of Compound 1 with the appropriate alkyl halide.

### Example 6

### Diazoalkane O-Alkylations

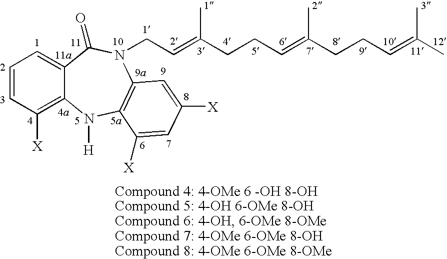

Compounds 4 and 5: 10-farnesyl-6,8-dihydroxy-4-methoxy-5,10-dihydro-dibenzo[b,e][1,4]diazepin-11-one and 10-farnesyl-4,8-dihydroxy-6-methoxy-5,10-dihydro-dibenzo[b,e][1,4]diazepin-11-one, are monomethylated have a calculated molecular weight of the major isotope of 476.27 g/mol and a formula of C₂₉H₃₆N₂O₄.

Compounds 6 and 7: 10-farnesyl-4-hydroxy-6,8-dimethoxy-5,10-dihydro-dibenzo[b,e][1,4]diazepin-11-one and 10-farnesyl-8-hydroxy-4,6-dimethoxy-5,10-dihydro-dibenzo[b,e][1,4]diazepin-11-one, are dimethylated and have a calculated molecular weight of the major isotope of 490.28 g/mol and a formula of C₃₀H₃₈N₂O₄.

Compound 8: 10-farnesyl-4,6,8-trimethoxy-5,10-dihydro-dibenzo[b,e][1,4]diazepin-11-one, is trimethylated and has a calculated molecular weight of the major isotope of 504.30 g/mol and a formula of C₃₁H₄₀N₂O₄.

All O-methylated compounds (4 to 8) were prepared and identified according to the following procedure:

a) Preparation:

Compound 1 (20.0 mg) in MeOH (2.0 mL) was treated with excess of CH₂N₂ in diethyl ether and the mixture stirred overnight. The resulting mixture was separated by preparative TLC (Merck Silica gel 60 F₂₅₄), using 2.5% MeOH in CHCl₃ as eluent. A mixture of Compounds 4 and 5 (1.0 mg), Compound 6 (0.5 mg), Compound 7 (5.5 mg) and Compound 8 (3.0 mg) were isolated with Rf value of 0.09, 0.35, 0.39 and 0.92 respectively.

b) Structural Elucidation:

The calculated molecular weights of the major isotopes (mono: 476.27, di: 490.28 and tri: 504.30) and formulae (mono: C₂₉H₃₆N₂O₄, di: C₃₀H₃₈N₂O₄ and tri: C₃₁H₄₀N₂O₄) respectively of mono methylated (Compounds 4 and 5), dimethylated (Compounds 6 and 7) and trimethylated (Compound 8) were confirmed by mass spectral (MS) analysis. MS of both Compounds 4 and 5 gave a (M−H)⁻ molecular ion of 475.5 by negative ionization and a (M+Na)⁺ molecular ion of 499.4 by positive ionization. MS of Compound 6 gave a (M−H)⁻ molecular ion of 489.5 by negative ionization and a (M+Na)⁺ molecular ion of 513.4 by positive ionization. MS of Compound 7 gave a (M−H)⁻ molecular ion of 489.5 by negative ionization and a (M+Na)⁺ molecular ion of 513.4 by positive ionization. MS of Compound 8 gave a (M−H)⁻ molecular ion of 503.6 by negative ionization and a (M+Na)⁺ molecular ion of 527.4 by positive ionization. Proton NMR spectral analysis for Compounds 4 to 8 is shown in Table 9. Signals were easily assigned based on comparison with the spectra of Compound 1.

TABLE 9
¹H NMR (δ_{H}, ppm) Data of Compounds 4, 5, 6, 7 and 8 in
MeOH-D₄

| Assignmen | 4 | 5 | 6 | 7 | 8 | Group |
|---|---|---|---|---|---|---|
| 1 | 7.28 | 7.17 | 7.19 | 7.28 | 7.29 | CH |
| 2 | 6.91 | 6.78 | 6.86 | 6.92 | 6.92 | CH |
| 3 | 7.03 | 6.86 | 6.79 | 7.03 | 7.03 | CH |
| 4 | 3.94 | N/D | N/D | 3.93 | 3.94 | C—X^{a} |
| 5 | N/D | N/D | N/D | N/D | 6.95 | NH |
| 6 | N/D | 3.87 | 3.91 | 3.87 | 3.89 | C—X^{b} |
| 7 | 6.28 | 6.23 | 6.46 | 6.33 | 6.45 | CH |
| 8 | N/D | N/D | 3.75 | N/D | 3.74 | C—X^{c} |
| 9 | 6.38 | 6.34 | 6.52 | 6.38 | 6.57 | CH |
| 1′ | 4.56 | 4.56 | 4.61 | 4.57 | 4.60 | CH₂ |
| 2′ | 5.36 | 5.36 | 5.38 | 5.34 | 5.31 | CH |
| 4′^{d} | 2.05 | 2.05 | 2.05 | 2.05 | 2.04 | CH₂ |
| 5′^{d} | 2.10 | 2.10 | 2.09 | 2.09 | 2.08 | CH₂ |
| 6′ | 5.09 | 5.09 | 5.07 | 5.08 | 5.06 | CH |
| 8′^{d} | 1.96 | 1.96 | 1.95 | 1.95 | 1.93 | CH₂ |
| 9′^{d} | 2.04 | 2.04 | 2.04 | 2.04 | 2.03 | CH₂ |
| 12′ | 1.66 | 1.66 | 1.65 | 1.66 | 1.65 | CH₃ |
| 10′ | 5.09 | 5.09 | 5.07 | 5.08 | 5.06 | CH |
| 1″ | 1.74 | 1.74 | 1.75 | 1.73 | 1.73 | CH₃ |
| 2″ | 1.60 | 1.60 | 1.60 | 1.59 | 1.59 | CH₃ |
| 3″ | 1.57 | 1.57 | 1.55 | 1.57 | 1.55 | CH₃ |

N/D: Not determined - not observed
^{a}X is OCH₃ in Compounds 4, 7 and 8; X is OH in Compounds 5 and 6
^{b}X is OCH₃ in Compounds 5, 6, 7 and 8; X is OH in Compound 4
^{c}X is OCH₃ in Compounds 6 and 8; X is OH in Compounds 4, 5 and 7
^{d}Signals of 4′, 5′, 8′ and 9′ are very close; assignement was based on Compound 1

### Example 7

### Acetic Anhydride O-Acylations

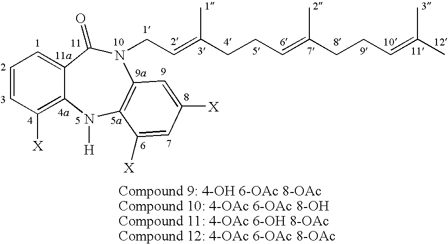

Compounds 9, 10 and 11: 6,8-diacetoxy-10-farnesyl-4-hydroxy-5,10-dihydro-dibenzo[b,e][1,4]diazepin-11-one; 4,6-diacetoxy-10-farnesyl-8-hydroxy-5,10-dihydro-dibenzo[b,e][1,4]diazepin-11-one and 4,8-diacetoxy-10-farnesyl-6-hydroxy-5,10-dihydro-dibenzo[b,e][1,4]diazepin-11-one, are diacetylated and have a calculated molecular weight of the major isotope of 546.27 g/mol and a formula of C₃₂H₃₈N₂O₆.

Compound 12: 4,6,8-triacetoxy-10-farnesyl-5,10-dihydro-dibenzo[b,e][1,4]diazepin-11-one, is triacetylated and has a calculated molecular weight of the major isotope of 588.28 g/mol and a formula of C₃₄H₄₀N₂O₇.

All acetylated compounds (9 to 12) were prepared and identified according to the following procedure:

a) Preparation:

Compound 1 (120.5 mg) was stirred overnight with acetic anhydride (720 μL, 29 eq, Aldrich) in presence of 6 drops of pyridine (Aldrich). The reaction mixtures submitted to HPLC separation. Purification by multiple injection on a Waters™ RCM Nova-Pak HR™ C18, 6 μm, 60A 25×200 mm column (20 mL/min H₂O/CH₃CN 80:30-70:75, 0-8 min; 30:70-0:100, 8-18 min and HPLC run for 20 min) gave Compound 11 (11.4 mg), Compound 10 (9.2 mg), Compound 9 (11.4 mg), Compound 12 (91.2 mg) with retention time of 16.2, 17.6, 18.0 and 18.5 min, respectively.

b) Structural Elucidation:

The calculated molecular weights of the major isotopes (di: 546.27 and tri: 588.28) and formulae (di: C₃₂H₃₈N₂O₆ and tri: C₃₄H₄₀N₂O₇) respectively of diacetylated (Compounds 9, 10 and 11) and triacetylated (Compound 12) were confirmed by mass spectral (MS) analysis. MS of Compound 9 gave a (M−H)-molecular ion of 545.6 by negative ionization and a (M+Na)⁺ molecular ion of 569.4 by positive ionization. MS of Compound 10 gave a (M−H)⁻ molecular ion of 545.6 by negative ionization and a (M+Na)⁺ molecular ion of 569.5 by positive ionization. MS of Compound 11 gave a (M−H)⁻ molecular ion of 545.5 by negative ionization and a (M+Na)⁺ molecular ion of 569.4 by positive ionization. MS of Compound 12 gave a (M−H)⁻ molecular ion of 587.6 by negative ionization and a (M+Na)⁺ molecular ion of 611.5 by positive ionization. Proton NMR spectral analysis for Compounds 9 to 12 is shown in Table 10. Signals were easily assigned based on comparison with the spectra of Compound 1.

TABLE 10
¹H NMR (δ_{H}, ppm) Data of Compounds 9, 10, 11 and 12 in
CDCl₃

| Assignmen | 9 | 10 | 11 | 12 | Group |
|---|---|---|---|---|---|
| 1 | 7.27 | 7.72 | 7.75 | 7.75 | CH |
| 2 | 6.67 | 6.99 | 7.02 | 7.10 | CH |
| 3 | 6.67 | 7.15 | 7.15 | 7.16 | CH |
| 4 | N/D | 2.42 | 2.42 | 2.41 | C—X^{a} |
| 5 | 6.45 | 5.90 | 6.29 | 6.15 | NH |
| 6 | 2.36 | 2.41 | N/D | 2.40 | C—X^{b} |
| 7 | 6.82 | 6.52 | 6.15 | 6.96 | CH |
| 8 | 2.28 | N/D | 2.25 | 2.27 | C—X^{c} |
| 9 | 6.95 | 6.67 | 6.55 | 6.84 | CH |
| 1′ | 4.60 | 4.57 | 4.57 | 4.58 | CH₂ |
| 2′ | 5.43 | 5.38 | 5.41 | 5.42 | CH |
| 4′^{d} | 2.08 | 2.06 | 2.05 | 2.06 | CH₂ |
| 5′^{d} | 2.09 | 2.08 | 2.09 | 2.09 | CH₂ |
| 6′ | 5.10 | 5.09 | 5.09 | 5.10 | CH |
| 8′^{d} | 1.98 | 1.97 | 1.98 | 1.98 | CH₂ |
| 9′^{d} | 2.08 | 2.06 | 2.05 | 2.06 | CH₂ |
| 10′ | 5.10 | 5.09 | 5.09 | 5.10 | CH |
| 12′ | 1.69 | 1.69 | 1.69 | 1.68 | CH₃ |
| 1″ | 1.72 | 1.70 | 1.71 | 1.72 | CH₃ |
| 2″ | 1.61 | 1.60 | 1.60 | 1.61 | CH₃ |
| 3″ | 1.60 | 1.60 | 1.60 | 1.60 | CH₃ |

N/D: Not determined - not observed
^{a}X is OAc in Compounds 10, 11 and 12; X is OH in Compound 9
^{b}X is OAc in Compounds 9, 10 and 12; X is OH in Compound 11
^{c}X is OAc in Compounds 9, 11 and 12; X is OH in Compound 10
^{d}Signals of 4′, 5′, 8′ and 9′ are very close; assignement was based on Compound 1

### Example 8

### Farnesyl Side Chain Modifications

### a) Synthesis and Structural Elucidation of Compound 46

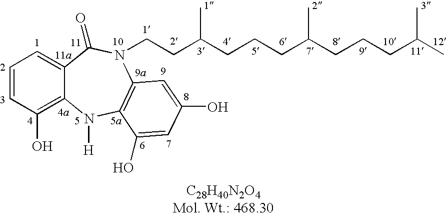

Compound 46: 10-(3,7,11-trimethyldodecyl)-4,6,8-trihydroxy-5,10-dihydro-dibenzo[b,e][1,4]diazepin-11-one, was prepared and identified according to the following procedure:

i) Preparation:

A solution of Compound 1 (51.1 mg in 3.0 mL MeOH) was stirred under hydrogen gas overnight in presence of platinum oxide (PtO₂, 10 mg, 0.4 eq) as a catalyst. The reaction mixture was filtered and purified by direct preparative HPLC using a Phenomenex Synergi™ MAX RP 21.2×200 mm column (20 mL/min, H₂O/CH₃CN gradient 30:70-30:70, 0-2 min; 30:70-0:100, 2-20 min). Fractions having a retention time of 12.8 min were combined to give 45.2 mg of Compound 46.

ii) Structural Elucidation:

Calculated molecular weight of the major isotope (468.30) and formula (C₂₈H₄₀N₂O₄) were confirmed by mass spectral analysis. Compound 46 mass spectra gave a (M−H)⁻ molecular ion of 467.4 by negative ionization and a (M+H)+molecular ion of 469.4 by positive ionization. Proton NMR spectral analysis of Compound 46 is shown in Table 11 below. Signals were easily assigned based on Compound 1 structure knowledge. As expected, aliphatic proton signals at positions 2′-11′ all have very close chemical shifts ranging from about 1 to 1.75 ppm (integrating for 17 protons), methyl protons at positions 12′ and 1″-3″ are all very close as well (shifts 0.8-0.95 ppm, integrating for 12 protons). These signals are also complex from the fact that 2 diastereomers of positions 3′ and 7′ are present in the mixture, and in different proportions. Labile protons were not observed since NMR was done in deuterated methanol.

### b) Synthesis and Structural Elucidation of Compound 78

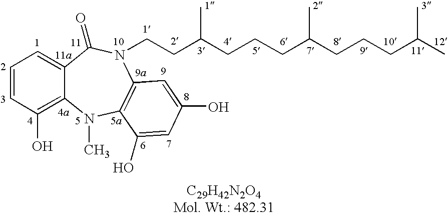

Compound 78, namely 10-(3,7,11-trimethyldodecyl)-4,6,8-trihydroxy-5-methyl-5,10-dihydro-dibenzo[b,e][1,4]diazepin-11-one, was prepared and identified as follows:

i) Preparation:

A solution of Compound 2 (23.7 mg) in MeOH (2.0 mL) was stirred under hydrogen gas overnight in presence of platinum oxide (PtO₂, 10 mg, catalyst) as a catalyst. The reaction mixture was filtered and concentrated in vacuo to give 21.6 mg of Compound 78.

ii) Structural Elucidation:

The calculated molecular weight (482.31) and formula (C₂₉H₄₂N₂O₄) of Compound 78 was confirmed by mass spectral analysis: negative ionization gave an (M−H)⁻ molecular ion of 481.3 and positive ionization gave an (M+H)+molecular ion of 483.3. The farnesyl olefinic protons on the NMR spectra were replaced by aliphatic proton signals in the region of around 0.76-1.86 ppm, integrating for 17 protons, 3CH, 7CH₂. The characteristic N-methyl group (5-N-Me) was easily assigned as shown in Table 11 below.

TABLE 11
¹H NMR (δ_{H}, ppm) Data of Compounds 46 and 78 in CD₃OD

| Assignment | Compound 46 | Compound 78 | Group |
|---|---|---|---|
| 1 | 7.15 | 7.18 | CH |
| 2 | 6.76 | 7.12 | CH |
| 3 | 6.84 | 7.01 | CH |
| 5-N-Me | N/A | 2.96, 2.95^{b} | CH₃ |
| 7 | 6.24 | 6.23 | CH |
| 9 | 6.26 | 6.33, 6.35^{b} | CH |
| 1′ | 4.16, 3.99 | 4.42, 3.86 | CH₂ |
| 2′-11′^{a} | ~1.00-1.75 | ~0.76-1.86 | 3CH,7CH₂ (17H) |
| 12′ and 1″-3″^{a} | ~0.8-0.95 | ~0.87-1.00 | 4CH₃ (12H) |

N/A: not applicable, group not present in the molecule
^{a}Signals are very close.
^{b}Mixture of isomers.

### c) Synthesis and Elucidation of Compounds 17 and 18 by Epoxidation

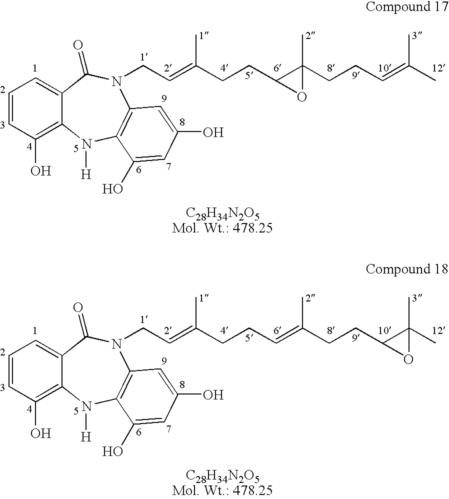

Compound 17: 10-(3,7,11-trimethyl-6,7-epoxydodeca-2,10-dienyl)-4,6,8-trihydroxy-5,10-dihydro-dibenzo[b,e][1,4]diazepin-11-one, and
Compound 18: 10-(3,7,11-trimethyl-10,11-epoxydodeca-2,10-dienyl)-4,6,8-trihydroxy-5,10-dihydro-dibenzo[b,e][1,4]diazepin-11-one, were prepared and identified according to the following procedure:

i) Preparation:

A mixture of Compound 1 (24.0 mg) and 3-chloroperbenzoic acid (mCPBA, 7.8 mg, 0.9 eq) in THF (1.0 mL) were stirred overnight at room temperature. The reaction mixture was diluted with MeOH (1.0 mL) and subjected to purification on Waters HPLC using a Photodiode Array detector. The mixture was purified by multiple injections on a Waters™ RCM Nova-Pak™ C-18 25×200 mm column (20 mL/min, H₂O/CH₃CN gradient 80:20-30:70, 0-8 min; 30:70-0:100, 8-20 min). Pure Compound 17 (2.11 mg) and Compound 18 (1.68 mg) were obtained by concentration in vacuo of the combined fractions respectively having retention time 11.2 min and 10.6 min.

ii) Structural Elucidation:

Calculated molecular weights of the major isotopes (478.25) and formulae (C₂₈H₃₄N₂O₅) were confirmed by mass spectral analysis. Compound 17 mass spectra gave a (M−H)⁻ molecular ion of 477.3 by negative ionization and a (M+H)⁺ molecular ion of 479.4 by positive ionization. Compound 18 mass spectra gave a (M−H)⁻ molecular ion of 477.3 by negative ionization and a (M+H)⁺ molecular ion of 479.4 by positive ionization. Proton NMR spectral analysis of Compounds 17 and 18 is shown in Table 12. Signals were easily assigned based on Compound 1 structure knowledge. As expected, epoxide protons signals were shifted upfield, compared to the alkene protons of Compound 1 (from 5.09 to 2.75 ppm for Compound 17, and from 5.06 to 2.73 ppm for Compound 18). Exchangeable protons were not observed as NMR was done in deuterated methanol.

TABLE 12
¹H NMR (δ_{H}, ppm) Data of Compounds 17 and 18 in CD₃OD

| Assignment | Compound 17 | Compound 18 | Group |
|---|---|---|---|
| 1 | 7.17 | 7.18 | CH |
| 2 | 6.77 | 6.77 | CH |
| 3 | 6.85 | 6.86 | CH |
| 7 | 6.22 | 6.23 | CH |
| 9 | 6.27 | 6.27 | CH |
| 1′ | 4.61, 4.54 | 4.55 | CH₂ |
| 2′ | 5.42 | 5.37 | CH |
| 4′ | 2.17 | 2.08^{a} | CH₂ |
| 5′ | 1.62, 1.42 | 2.13^{a} | CH₂ |
| 6′ | 2.75 | 5.16 | CH |
| 8′ | 1.62, 1.42 | 2.08^{a} | CH₂ |
| 9′ | 2.09 | 1.60 | CH₂ |
| 10′ | 5.09 | 2.73 | CH |
| 12′ | 1.67 | 1.26 | CH₃ |
| 1″ | 1.77 | 1.74 | CH₃ |
| 2″ | 1.26 | 1.64 | CH₃ |
| 3″ | 1.60 | 1.20 | CH₃ |

^{a}Signals are very close, and are interchangeable

### d) Synthesis and Structural Elucidation of Compound 89 and 92

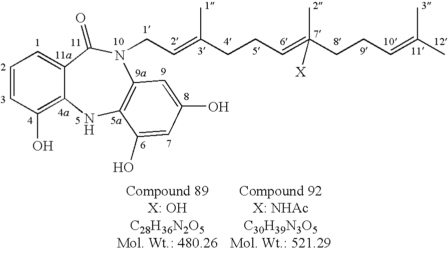

Compound 89, namely 10-(7-hydroxy-3,7,11-trimethyldodeca-2,10-dienyl)-4,6,8-trihydroxy-5,10-dihydro-dibenzo[b,e][1,4]diazepin-11-one, and

Compound 92, namely 10-(7-acetamido-3,7,11-trimethyldodeca-2,10-dienyl)-4,6,8-trihydroxy-5,10-dihydro-dibenzo[b,e][1,4]diazepin-11-one, were prepared and identified as follows:

i) Preparation:

Compound 1 (20.0 mg) was dissolved in CH₃CN (2.0 mL) and water (50 μL) and PTSA (56.0 mg) was added. The solution was stirred under reflux for 30 min. The reaction mixture was filtered and the filtrate subjected to Waters HPLC purification (multiple injections on Nova-Pack™ C-18 25×200 mm column: 20 mL/min, H₂O/CH₃CN gradient 70:30-20:80, 0-4 min; 20:80-0:100, 4-9 min), to give Compound 89 (0.73 mg, RT 10.0 min) and Compound 92 (0.33 mg, RT 10.5 min).

ii) Structure Elucidation of Compounds 89 and 92:

The calculated molecular weight of the major isotope (480.26) and formula (C₂₈H₃₆N₂O₅) of Compound 89 was confirmed by mass spectral analysis: negative ionization gave an (M−H)⁻ molecular ion of 479.8 and positive ionization gave an (M+H−H₂O)⁺ molecular ion of 464.1. The characteristic side chain signal (signal 6′) aliphatic methylene was easily assigned as shown in Table 13 below.

The calculated molecular weight of the major isotope (521.29) and formula (C₃₀H₃₉N₃O₅) of Compound 92 was confirmed by mass spectral analysis: negative ionization gave an (M−H)⁻ molecular ion of 522.8 and positive ionization gave an (M+H)⁺ molecular ion of 522.9. The characteristic side chain (signal 6′) aliphatic methylene and the acetamide (signal 7′-NHAc) were easily assigned as shown in Table 13 below.

TABLE 13
¹H NMR (δ_{H}, ppm) Data of Compounds 89 and 92 in CD₃OD

| Assignment | Compound 89 | Compound 92 | Group |
|---|---|---|---|
| 1 | 7.18 | 7.17 | CH |
| 2 | 6.77 | 6.77 | CH |
| 3 | 6.86 | 6.85 | CH |
| 7 | 6.23 | 6.22 | CH |
| 9 | 6.29 | 6.31 | CH |
| 1′ | 4.56 | 4.54 | CH₂ |
| 2′ | 5.39 | 5.40 | CH |
| 4′ | 2.05 | 2.05 | CH₂ |
| 5′, 6′, 8′, 9′ | 1.49-1.27^{a} | N/A | 4(CH₂) |
| 5′, 6′, 8′ | — | 1.77-1.40^{b} | 3(CH₂) |
| 7′-X | — | 1.92 | X^{c} |
| 9′ | — | 1.93 | CH₂ |
| 10′ | 5.13 | 5.12 | CH |
| 12′ | 1.68 | 1.67 | CH₃ |
| 1″ | 1.73 | 1.71 | CH₃ |
| 2″ | 1.14 | 1.23 | CH₃ |
| 3″ | 1.61 | 1.59 | CH₃ |

N/A: not applicable, group not present in the molecule
^{a}Signals 5′, 6′, 8′ and 9′ of Compound 89 are all very close.
^{b}Signals 5′, 6′ and 8′ of Compound 92 are all very close.
^{c}In Compound 89, X is OH, in Compound 92, X is NHC(O)CH₃.

e) Synthesis and Structural Elucidation of Compounds 95 and 96 by ozonolysis

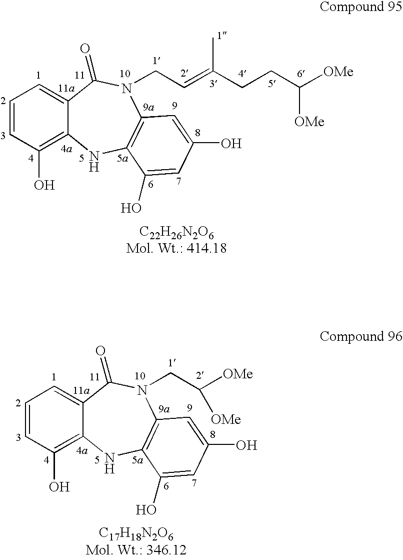

Compound 95, namely 10-(6,6-dimethoxy-3-methyl-2-hexenyl)-4,6,8-trihydroxy-5,10-dihydro-dibenzo[b,e][1,4]diazepin-11-one, and

Compound 96, namely 10-(6,6-dimethoxyethyl)-4,6,8-trihydroxy-5,10-dihydro-dibenzo[b,e][1,4]diazepin-11-one, were prepared and identified as follows:

i) Preparation:

Compound 1 (201.2 mg) was dissolved in MeOH (3.0 mL) and 03 (ozone) was bubbled in the solution for 2 min at −80° C. (dry ice/acetone). Dimethyl sulfide (146 ml) was added and the reaction mixture was warmed up and stirred at room temperature for 24 hrs. The reaction mixture was filtered and the filtrate subjected to purification on a Waters Auto-Purification System (multiple injections on YMC-Pack ODS-AQ column 20×250 mm: 20 mL/min, H₂O/CH₃CN gradient: 75:25 isocratic 3 min, 75:25-5:95, 3-30 min; 5:95-0:100, 30-31 min and 100% CH₃CN isocratic for 5 min), to give Compound 95 (0.96 mg, RT 12.4 min) and Compound 96 (1.23 mg, RT 8.7 min).

ii) Structural Elucidation of Compounds 95 and 96:

The calculated molecular weight of the major isotope (414.18) and formula (C₂₂H₂₆N₂O₆) of Compound 95 was confirmed by mass spectral analysis: negative ionization gave an (M−H)⁻ molecular ion of 413.4 and positive ionization gave an (M+Na)⁺ molecular ion of 437.6. The characteristic farnesyl side chain proton NMR signals (7′ to 11′, and 2″, 3″) were replaced by an aliphatic carbon (signal 6′) and two methoxy groups (6‘-OMe’s), easily assigned as shown in Table 14 below.

The calculated molecular weight of the major isotope (346.12) and formula (C₁₇H₁₈N₂O₆) of Compound 96 was confirmed by mass spectral analysis: negative ionization gave an (M−H)⁻ molecular ion of 345.2 and positive ionization gave an (M+Na)⁺ molecular ion of 369.3. The characteristic farnesyl side chain proton NMR signals were replaced by aliphatic carbon (signal 2′) and two methoxy groups (2‘-OMe’s), easily assigned as shown in Table 14 below.

TABLE 14
¹H NMR (δ_{H}, ppm) Data of Compounds 95 and 96 in CD₃OD

| Assignment | Compound 95 | Compound 96 | Group |
|---|---|---|---|
| 1 | 7.18 | 7.15 | CH |
| 2 | 6.77 | 6.77 | CH |
| 3 | 6.86 | 6.86 | CH |
| 7 | 6.23 | 6.24 | CH |
| 9 | 6.27 | 6.41 | CH |
| 1′ | 4.58 | 4.64 | CH₂ |
| 2′ | 5.35 | 4.72 | CH |
| 2′-OMe | N/A | 3.40 | 2 × OCH₃ |
| 4′ | 2.07 | N/A | CH₂ |
| 5′ | 1.66 | N/A | CH₂ |
| 6′ | 4.22 | N/A | CH |
| 6′-OMe | 3.37 | N/A | 2 × OCH₃ |
| 1″ | 1.74 | N/A | CH₃ |

N/A: not applicable, group not present in the molecule

### Example 9

### Aromatic Substitution Reaction

a) Synthesis and Structural Elucidation of Compound 97 by bromination

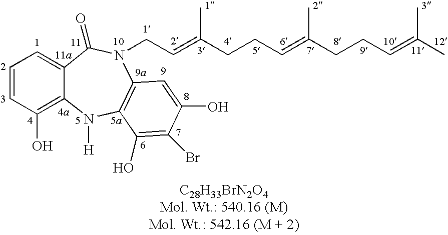

Compound 97: 10-(farnesyl)-7-bromo-4,6,8-trihydroxy-5,10-dihydro-dibenzo[b,e][1,4]diazepin-11-one, was prepared and identified according to the following procedure:

i) Preparation:

Compound 1 (116.0 mg) and N-bromosuccinimide (NBS, 45.5 mg) were dissolved in tetrahydrofuran (THF, 3.0 mL) and stirred at room temperature for 4 days. The reaction mixture was filtered and subjected to Waters HPLC purification (Nova-Pack™ C-18 25×200 mm column: 20 mL/min, H₂O/CH₃CN gradient 80:20-30:70, 0-8 min; 30:70-0:100, 8-18 min) to give Compound 97 (13.6 min) together with some impurities. The semi-purified sample was further purified by HPLC (Symmetry™ C-18 25×100 mm column: 20 mL/min, H₂O/CH₃CN gradient 70:30-30:70, 0-15 min), to give Compound 97 (9.5 mg, RT 13.0 min).

ii) Structural Elucidation:

The calculated molecular weight of the major isotopes (540.16 and 542.16) and formula (C₂₈H₃₃BrN₂O₄) of Compound 97 was confirmed by mass spectral analysis: negative ionization gave (M−H)⁻ molecular ions of 539.2 and 541.1, and positive ionization gave (M+H)⁺ molecular ions of 541.3 and 543.2. The presence of the two molecular ions in each mass spectrum confirmed the presence of a bromine group in the molecule. The structure was further confirmed by the absence of the aromatic (7) signal in the proton NMR spectrum as shown in Table 15 below.

TABLE 15
¹H NMR (δ_{H}, ppm) Data of Compound 97 in CD₃OD

| Assignment | Compound 97 | Group |
|---|---|---|
| 1 | 7.18 | CH |
| 2 | 6.79 | CH |
| 3 | 6.87 | CH |
| 9 | 6.49 | CH |
| 1′ | 4.56 | CH₂ |
| 2′ | 5.34 | CH |
| 4′ | 2.06 | CH₂ |
| 5′ | 2.09 | CH₂ |
| 6′ | 5.10 | CH |
| 8′ | 1.96 | CH₂ |
| 9′ | 2.04 | CH₂ |
| 10′ | 5.08 | CH |
| 12′ | 1.66 | CH₃ |
| 1″ | 1.74 | CH₃ |
| 2″ | 1.60 | CH₃ |
| 3″ | 1.57 | CH₃ |

### b) Synthesis and Structural Elucidation of Compound 99

Compound 2 (116.0 mg) and N-bromosuccinimide were dissolved in tetrahydrofuran (THF, 3.0 mL) and stirred at room temperature for 4 days. The reaction mixture was filtered and subjected to Waters HPLC purification to give Compound 99.

### Example 10

### Synthesis and Structural Elucidation of Compound

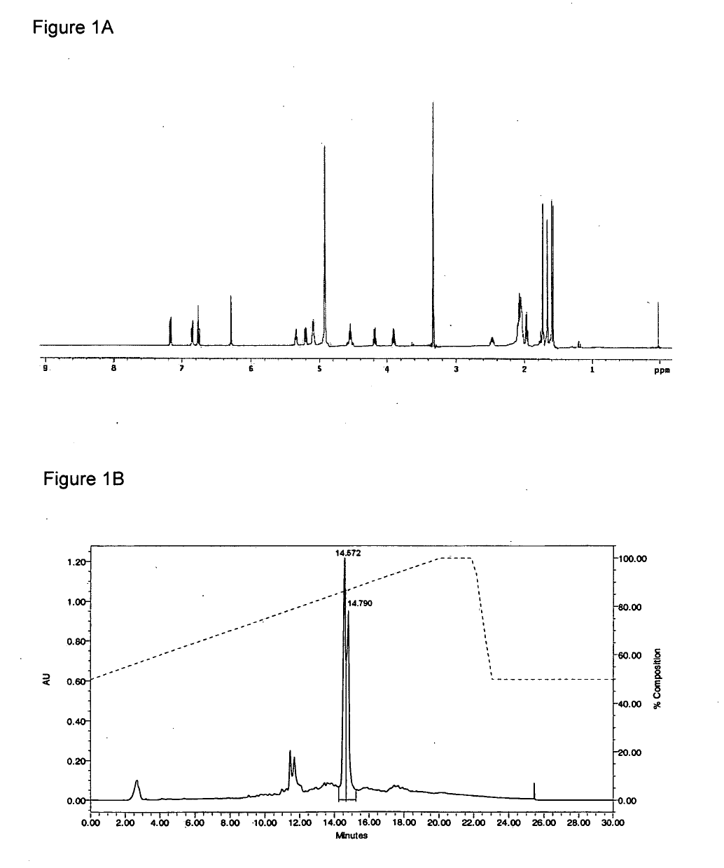

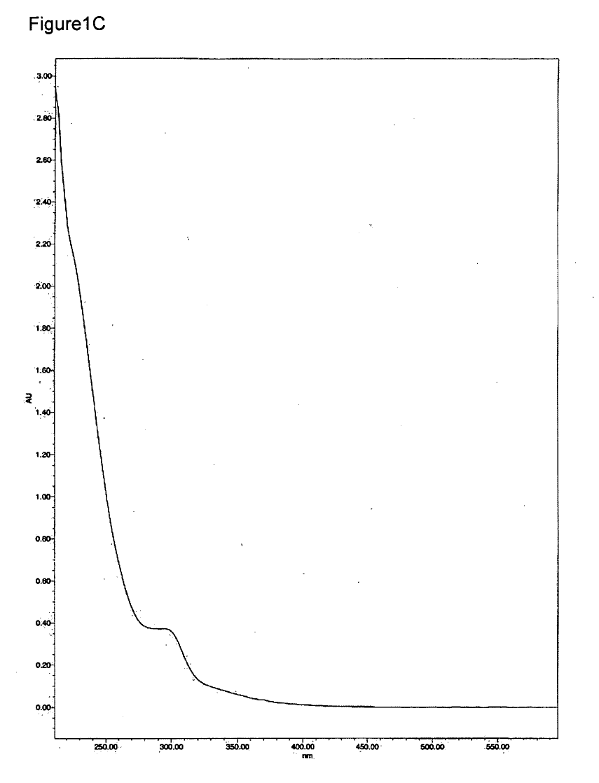

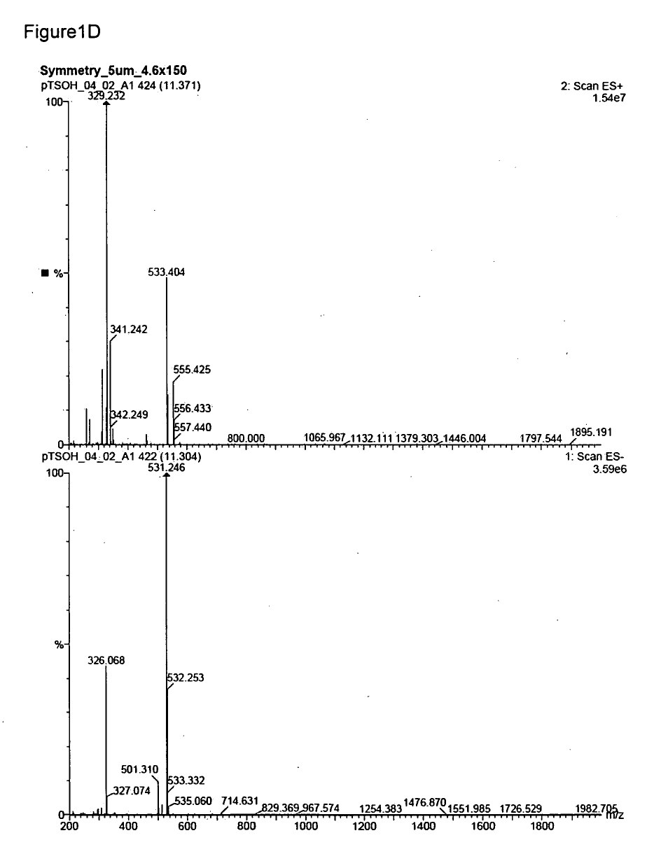

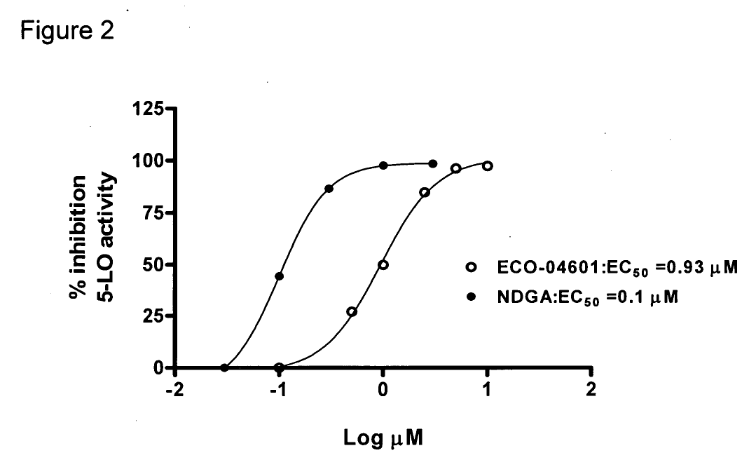

A mixture of Compound 1 (ECO-04601) (500 mg) and p-TSOH (400 mg) in THF (40 mL) was refluxed for 6 h. The reaction mixture was allowed to cool and subjected for purification on a Waters HPLC with Photodiode detector. The THF derivative was obtained by the multiple injections on an ACE C18 21.2×250 mm column (flow rate 20 mL/min, solvent H₂O/CH₃CN gradient 50:50 to 0:100, 0-20 min). Compound 100 (ECO-04687) was eluted at 14.8 min. Pooled fractions from multiple chromatographies were concentrated yield an off-white powder (142 mg). UV λₘₐₓ 220, 294 nm. ¹H NMR, see NMR spectrum (ECO-4601_MODI_p-TSOH_{—}04_{—}02_CD₃OD) in FIG. 1A-D.

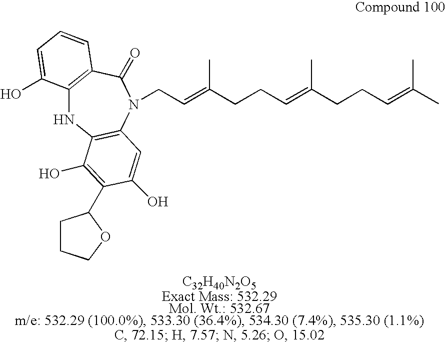

### Example 11

### Dibenzodiazepinone Analogs General Procedures

### a) O-Alkylation:

Alkylation Compounds 4-8 are also produced using the procedure presented in Example 6. Compounds 38 and 39 are also produced using the procedure of Example 6, by controlling the amount of diazomethane, the reaction temperature and/or the reaction time. Compound 38 is also prepared in two steps, from Compound 10, using the procedure of Example 6, the resulting mono-methyl-diacetate compound is subsequently hydrolyzed using aqueous acidic or basic (mild) conditions to obtain Compound 38. Compounds 4, 5, 6, 7 and 39 are also prepared in a similar two-step procedure, when using the appropriate Compound as starting material, which are respectively Compounds 9,11, 35, 37 and 36.

### Syntheses of Compounds 6, 7 and 38 by Methylation of Compound I

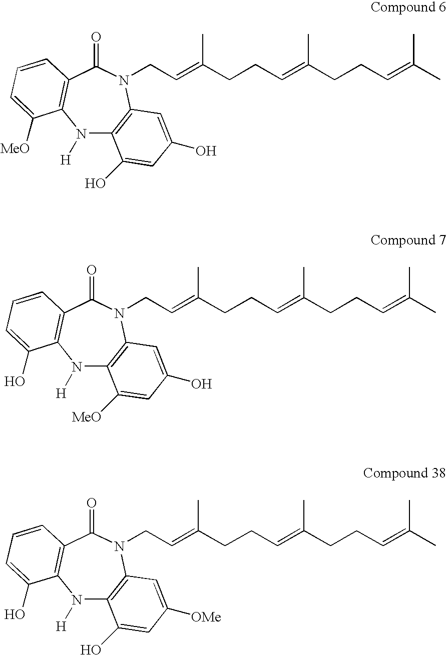

A solution of Compound 1 (1 g) in tetrahydrofuran 50 (ml) is treated with 1.5 equivalents of diazomethane. The mixture is heated under reflux for one hour, cooled to room temperature and poured into a mixture of toluene (200 ml) and water (200 ml). The layers are separated and the aqueous layer is extracted once more with an equal portion of toluene. The combined toluene layers are washed once with 1 N aqueous acetic acid and then concentrated to a crude product, which is predominantly a mixture of Compounds 6, 7 and 38 with some unchanged starting material and over-methylated derivatives. The desired products may be separated and purified by HPLC or HSCC chromatography or preparative TLC, using the systems as described in any of Examples 2 and 4-9 above, to obtain approximately 200 mg of each of Compounds 6, 7 and 38.

### b) O-Acylations:

O-acetylated compounds 35-37 are also produced using the procedures presented in Example 7, using a lower quantity of acetic anhydride, lower temperature, and/or shorter reaction times.

### Syntheses of Compounds 35, 36 and 37 by Esterification of Compound 1.

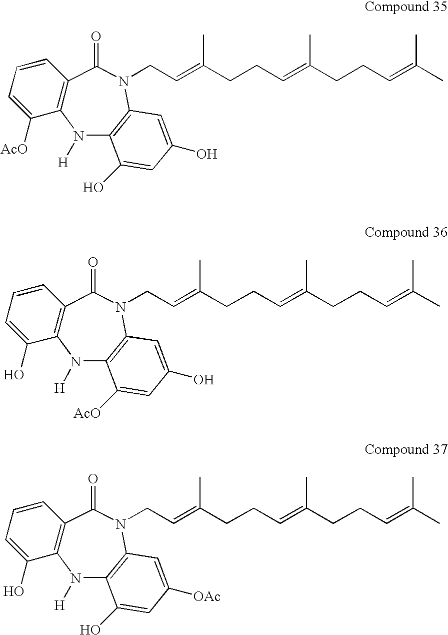

To a solution of Compound 1 dissolved in toluene (9 parts) tetrahydrofuran (1 part), cooled in an ice-bath is added 1.1 equivalents of acetic anhydride and two drops of boron trifluoride etherate. The reaction is maintained cool in an ice bath and stirred at 0° C. for 1-2 hours. The reaction mixture is then poured into aqueous 5% sodium bicarbonate solution shaken and the toluene layer is removed. The aqueous laer is re-extracted with toluene and the combined toluene layers are concentrated to a mixture of predominantly Compounds 35, 36 and 37, contaminated with some unchanged starting material and some diacetates. Compounds 35, 36 and 37 are separated and purified by HPLC or HSCC using one of the systems described in Examples 2 and 4-9. In a typical experiment yields of 25% to 30% are obtained for each of Compounds 35, 36 and 37.

Compounds 9-12 are also produced using the same procedure, with appropriate numbers of molar equivalents (2.2 and 3.3).

### c) N-alkylations:

N-Alkylations are accomplished using either an alkyl halide (iodide, bromide, chloride) or another alkylating agent, such as a dialkylsulfate, or an alkylsulfonate (triflate, mesylate, tosylate, and the like). Compounds 2, 3, 14, and 60 to 77 are also produced using the procedures exemplified in Examples 4 and 5.

### Syntheses of Compounds 2, 3 and 14 by N-Alkylation of Compound 1.

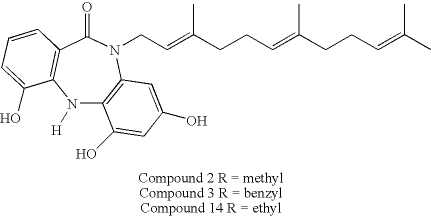

To a solution of Compound 1 (50 mg) dissolved in an excess of the appropriate alkyl halide (iodomethane for Compound 2, benzyl chloride for Compound 3 or ethyl bromide for Compound 14) is added a few drops of pyridine (catalytic amount). The reaction mixture is stirred for 72 hours, or until completion, and then evaporated to dryness under reduced pressure to obtain Compound 2, 3 or 14 respectively, in an essentially pure form in an almost quantitative yield. The crude compound is further purified by HPLC or Preparative TLC, using the procedures described in Examples 2 and 4-9.

Compounds 60 to 77 are also prepared via this procedure, or the procedures of Example 5, by reaction of Compound 1 respectively with 3-chloro-1-butene, 1-chloro-2-methylpropane, crotylchloride, 1-bromopropane, 1-bromobutane, 1-bromo-2-methylbutane, 2-chloro-2-methylpropane, 1-bromohexane, 1-chlorooctane, trifluoromethyl iodide, heptafluoro-1-iodopropane, heptafluoro-2-iodopropane, 2-iodo-1,1,1-trifluoroethane, bromocyclopropane, 1-chloro-3-phenylpropane, and 2-bromobutane. Compound 78 is also prepared by this procedure, by reacting Compound 46 with iodomethane.

Compounds 60 to 77 are also prepared by the procedures of Example 4, by reaction of Compound 1 with their respective dialkylsulfate (or alkylsulfonate), which is either commercially available or can be prepared, for example by the reaction of the appropriate alcohol with a activated sulfate or sulfonate (e.g. chloride, anhydride, and the like). As an example, 1-hexane triflate is prepared just prior to use by the reaction of 1-hexanol with trifluoromethanesulfonic anhydride (Tf₂O) in tetrahydrofuran, using an equimolar amount (vs Tf₂O) of base, such as triethylamine. The reaction is worked up by careful treatment with water (containing 1% triethylamine), extracted with ether, dried with magnesium sulfate and concentrated in vacuo. Other examples of procedures for the preparation of alkyl sulfates and sulfonates are described in Advanced Organic Chemistry, Jerry March, supra (e.g. page 404).

### d) N-Acylation:

### Synthesis of Compound 13 by N-Acetylation of Compound 1

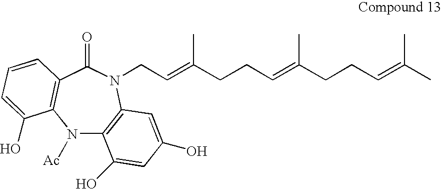

To a solution of Compound 1 dissolved in tetrahydrofuran (THF) is added 1.2 equivalents of acetyl chloride and a few drops of pyridine. The reaction mixture is allowed to stand at room temperature for 1-2 hours and then evaporated to dryness under reduced pressure to obtain a crude mixture containing Compound 13. Compound 13 is purified using HPLC or preparative TLC plates and the procedure described in any one of Examples 2, and 4-9.

### e) Peracetylation:

### Syntheses of Compound 15 by Peracetylation of Compound 1.

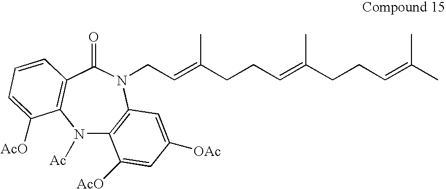

A solution of Compound 1 (100 mg) in acetic anhydride (5 ml) is treated with pyridine (250 ul). The reaction mixture is allowed to stand overnight at room temperature and is then diluted with toluene (100 ml). The toluene solution is washed well with aqueous 5% sodium bicarbonate solutions, then with water and is finally concentrated under reduced pressure to give an essentially pure sample of Compound 15 in almost quantitative yield.

### f) Epoxidations:

The epoxide compounds of the present invention (e.g., compounds according to exemplary Compounds 16-22 are made from Compound 1, and Compounds 23 to 34 from the appropriate starting material, by treatment with any of a number of epoxidizing reagents such as perbenzoic acid, monoperphthalic acid or more preferably by m-chloroperbenzoic acid in an inert solvent such as tetrahydrofuran. It will be appreciated by one of ordinary skill in the art that slightly greater than one molecule equivalent of epoxidizing agent will result in the maximal yield of mono-epoxides, and that the reagent, solvent, concentration and temperature of the reaction will dictate the ratio of specific mono-epoxides formed. It will also be appreciated that the mono-epoxides will be enantiomeric mixtures, and that the di-epoxides and the tri-epoxide can be prepared as diastereomers and that the conditions of the reaction will determine the ratios of the products. One skilled in the art will appreciate that under most conditions of reactions the product will be a mixture of all possible epoxides and that these may be separated by standard methods of chromatography. Exemplary approaches to the generation of mono-, di-, and tri-epoxides are provided below.

1) Mono-epoxides Compounds, 16, 17 and 18 prepared by Epoxidation of Compound I (as also shown in Example 8(c)):

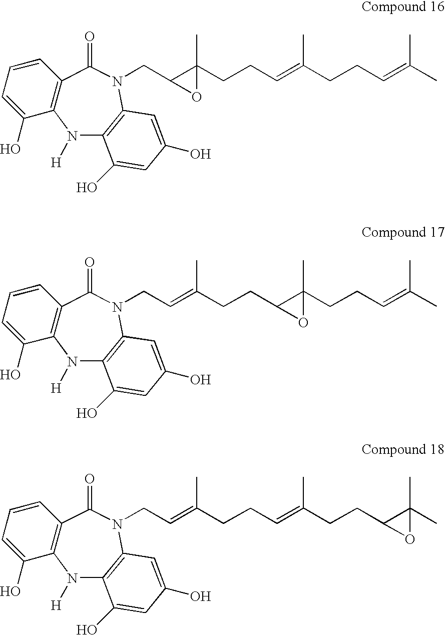

To a solution of Compound 1 dissolved in tetrahydrofuran (THF) is added 1.1 equivalents of meta-chloroperbenzoic acid. The reaction is cooled in an ice bath and stirred at 0° C. for 1-2 hours. The reaction mixture is then evaporated to dryness, re-dissolved in methanol and subjected to liquid chromatography on a column of Sephadex™ LH-20 to isolate a mixture of predominantly Compounds 16, 17 and 18, contaminated with some unchanged starting material and some di- and tri-epoxides. Compounds 16, 17 and 18 are separated and purified by HPLC using the system described in Examples 2 and 4-9. In a typical experiment yields of 15% to 25% are obtained for each of Compounds 16, 17 and 18.

### 2) Synthesis of Compounds 19, 20 and 21 by Di-Epoxidation of Compound 1:

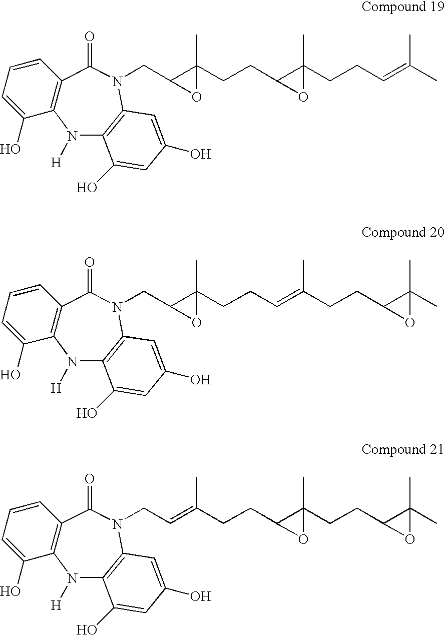

To a solution of Compound 1 dissolved in tetrahydrofuran (THF) is added 2.3 equivalents of meta-chloroperbenzoic acid. The reaction is cooled in an ice bath and stirred at 0° C. for 1-2 hours. The reaction mixture is then evaporated to dryness, re-dissolved in methanol and subjected to liquid chromatography on a column of Sephadex™ LH-20 to isolate a mixture of predominantly Compounds 19, and 21, contaminated with traces of unchanged starting material and some mono- and tri-epoxides. Compounds 19, 20 and 21 are separated and purified by HPLC using the system described in Examples 2 and 4-9. In a typical experiment, yields of 15% to 20% are obtained for each of Compounds 19, 20 and 21.

### 3) Synthesis of Compound 22 by Tri-Epoxidation of Compound 1:

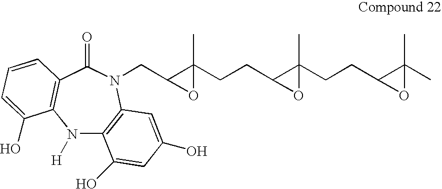

To a solution of Compound 1, dissolved in tetrahydrofuran (THF), is added 3.5 equivalents of meta-chloroperbenzoic acid. The reaction is cooled in an ice bath and stirred at 0° C. for 1-2 hours. The reaction mixture is then evaporated to dryness, re-dissolved in methanol and subjected to liquid chromatography on a column of Sephadex™ LH-20 to isolate Compound 22 in an essentially pure form in a yield of 80+%.

4) Syntheses of Compounds 23 and 24 by epoxidation of Compound 42.

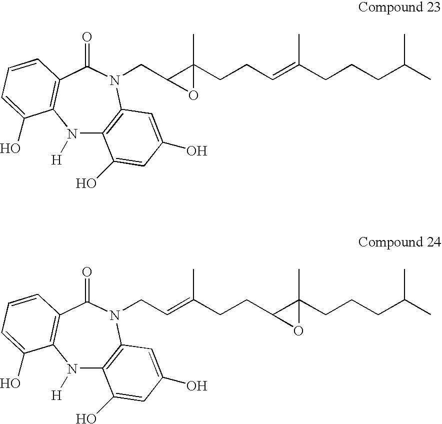

To a solution of Compound 42 dissolved in tetrahydrofuran (THF) is added 1.1 equivalents of meta-chloroperbenzoic acid. The reaction is cooled in an ice bath and stirred at 0° C. for 1-2 hours. The reaction mixture is then evaporated to dryness, re-dissolved in methanol and subjected to liquid chromatography on a column of Sephadex™ LH-20 to isolate a mixture of predominantly Compounds 23 and 24, contaminated with some unchanged starting material and some diepoxide. Compounds 23 and 24 are separated and purified by HPLC or HSCC using one of the systems described in Examples 2 and 4-9. In a typical experiment yields of 35% to 40% are obtained for each of Compounds 23 and 24.

Compounds 25 and 29 to 34 are prepared using this procedure. In each procedure, Compound 42 is replaced by the appropriate starting material. More specifically, Compounds 25 and 29 are prepared using Compound 41 as starting material; Compounds 30 and 31 are prepared using Compound 40 as starting material; and Compounds 32, 33, and 34 are prepared respectively from Compounds 45, 44 and 43.

### 5) Synthesis of Compound 28 by Epoxidation of Compound 40.

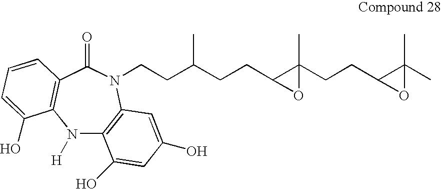

To a solution of Compound 40 dissolved in tetrahydrofuran (THF) is added 2.2 equivalents of meta-chloroperbenzoic acid. The reaction is cooled in an ice bath and stirred at 0° C. for 1-2 hours. The reaction mixture is then evaporated to dryness, re-dissolved in methanol and subjected to liquid chromatography on a column of Sephadex™ LH-20 to isolate essentially pure Compound 28 in good yield.

Compounds 26 and 27 are prepared using the same procedure, but using respectively from Compounds 42 and 41 as starting material, instead of Compound 40.

### g) Epoxide Opening:

### Syntheses of Compound 53 by Opening the Epoxide of Compound 16.

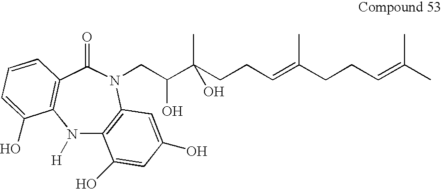

A solution of Compound 16 (100 mg) in tetrahydrofuran (50 ml) is treated with 1 N aqueous hydrochloric acid (5 ml). The reaction mixture is stirred overnight at room temperature and is then diluted with toluene (100 ml) and water (200 ml). The toluene layer is separated and the aqueous layer is extracted with a further 100 ml of toluene. The combined toluene layers are washed once more with water (50 ml) and the separated and dried under vacuum to give the vicinal glycol Compound 53. The same procedure is used to prepare Compounds 54 to 59, using respectively Compounds 17 to 22 as starting material.

### h) Hydrogenation:

Compounds 40 to 46 (from Compound 1) and 78 (from Compound 2) are produced by catalytic hydrogenation using a source of hydrogen (hydrogen, formic acid, and the like), and a catalyst (palladium on charcoal, platinum oxide, Raney-Nickel, and the like). Hydrogen uptake is optionally measured or controlled.

### 1) Syntheses of Compounds 40, 41 and 42 by Hydrogenation of Compound 1.

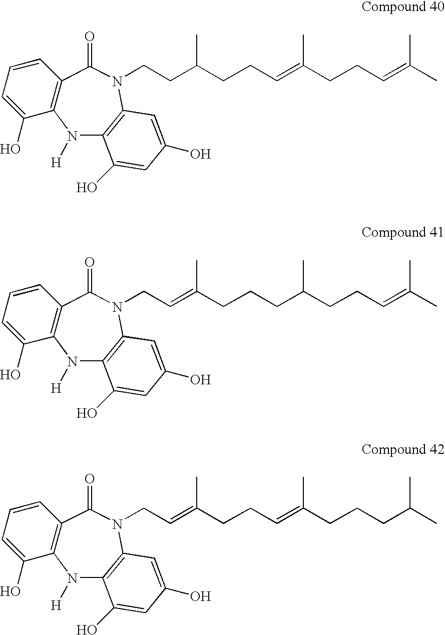

A solution Compound 1 (462 mg) in ethanol (200 ml) with palladium on charcoal (25 mg of 5%) is shaken in an hydrogenation apparatus in an atmosphere of hydrogen. The uptake of hydrogen by the reaction is measured carefully and at the point where one millimole of hydrogen has been consumed, shaking is stopped, the vessel is rapidly evacuated and the atmosphere is replaced with nitrogen. The catalyst is removed by filtration and the filtrate is concentrated to obtain a crude mixture of Compounds 40, 41 and 42 contaminated by unreacted starting material and minor amounts of over reduced products. The desired products may be separated and purified by HPLC or HSCC chromatography using the systems as described in Examples 2 and 4-9 above, to obtain approximately 100 mg of each of Compounds 40, 41 and 42.

### 2) Syntheses of Compounds 43, 44 and 45 by Hydrogenation of Compound 1.

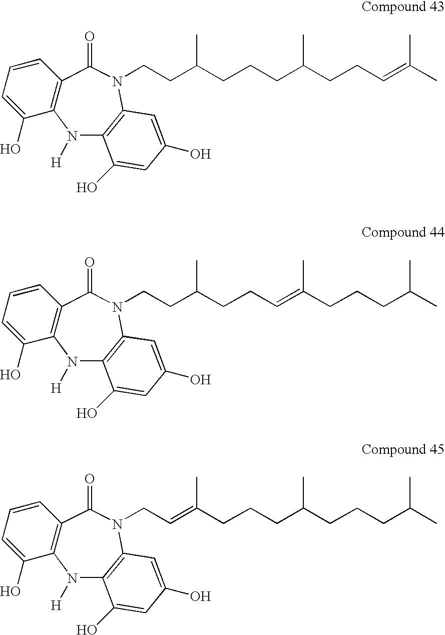

A solution of Compound 1 (462 mg) in ethanol (200 ml) with palladium on charcoal (25 mg of 5%) is shaken in an hydrogenation apparatus in an atmosphere of hydrogen. The uptake of hydrogen by the reaction is measured carefully and at the point where two millimoles of hydrogen has been consumed, shaking is stopped, the vessel is rapidly evacuated and the atmosphere is replaced with nitrogen. The catalyst is removed by filtration and the filtrate is concentrated to obtain a crude mixture of Compounds 43, 44 and 45 contaminated by trace amounts unreacted starting material and minor amounts of under and over reduced products. The desired products may be separated and purified by HPLC or HSCC chromatography using the systems as described in Examples 2 and 4-9 above, to obtain approximately 100 mg of each of Compounds 43, 44 and 45.

3) Syntheses of Compound 46 by Hydrogenation of Compound I (also Produced using the Procedure of Example 8(a) and 8(b)).

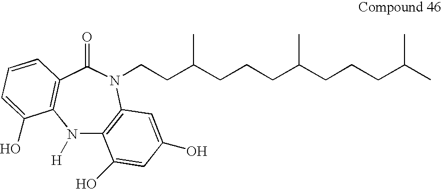

A solution of Compound 1 (462 mg) in ethanol (200 ml) with palladium on charcoal (25 mg of 5%) is shaken in an hydrogenation apparatus in an atmosphere of hydrogen. The uptake of hydrogen by the reaction is measured carefully and at the point where three millimoles of hydrogen have been consumed, shaking is stopped, the vessel is rapidly evacuated and the atmosphere is replaced with nitrogen. The catalyst is removed by filtration and the filtrate is concentrated to obtain an essentially pure sample of Compound 46.

Compound 78 is prepared from the same procedure, using Compound 2 as starting material, instead of Compound 1.

### i) Ozonolysis:

### Syntheses of Compounds 47, 49 and 51 by Ozonolysis of Compound 1.

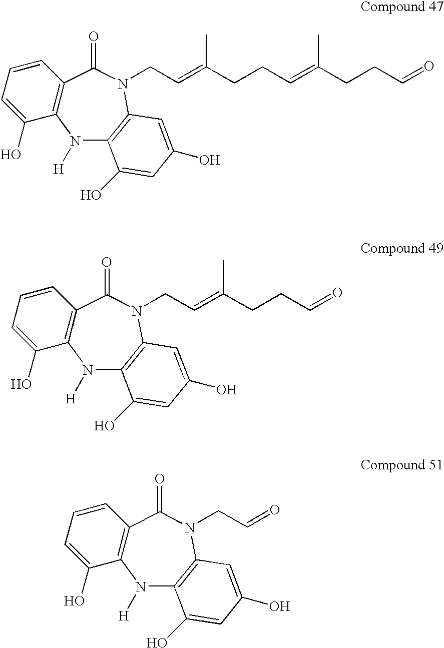

A solution of Compound 1 (462 mg) in dry ethyl acetate (200 ml) in an ozonolysis apparatus is cooled to below −20° C. A stream of ozone containing oxygen is passed into the solution from an ozone generator, which has been precalibrated such that the rate of ozone generation is known. To obtain predominantly Compound 47 the passage of ozone is halted after 0.9 millimole have been generated. To obtain predominantly Compound 49 the ozone passage is halted after 2 millimoles have been generated and to obtain Compound 51 as the predominant product 3.3 millimoles of ozone are generated.

At the completion of the ozonolysis, the reaction mixture is transferred to an hydrogenation apparatus, 5% palladium on calcium carbonate catalyst (0.2 g) is added to the reaction mixture which is maintained at less than −20° C. and is hydrogenated. When hydrogen uptake is complete the hydrogen atmosphere is replaced with nitrogen and the reaction mixture is allowed to come to room temperature, filtered to remove catalyst and the filtrate is concentrated. The crude product may be purified by chromatography using either HPLC or HSCC with the systems as described in Examples 2 and 4-9 to give, dependent on the amount of ozone used, Compounds 47, 49 and 51.

Dimethyl acetal compounds, for example, Compounds 94 to 96, are also produced by ozonolysis in methanol, followed by treatment with dimethyl sulfide. Aldehyde Compounds 47, 49 and 51, are obtained by hydrolysis (standard aqueous acidic conditions) of Compounds 94 to 96.

### j) Reduction:

Synthesis of Compound 48 by reduction of the aldehyde of Compound 47

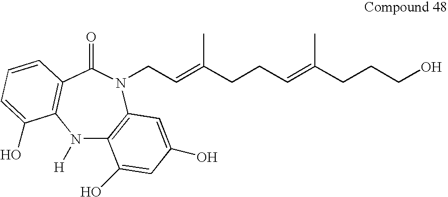

A solution of Compound 47 (50 mg) in isopropanol (5 ml) is cooled in an ice-salt bath and sodium borohydride (10 mg) is added and the mixture is stirred for 20 minutes. It is then diluted with water (20 ml) and extracted twice with toluene (10 ml portions) at ambient temperature. The combined toluene extracts are filtered and the filtrate is concentrated to give Compound 48.

Compounds 50 and 52 are produced by the same procedure, when replacing Compound 47 by Compounds 51 and 53 respectively as starting material.

### Example 12

### Pharmacological Activity Profile

Compound 1 and Compounds 2 to 12 and Compound 46 were tested for binding against a variety of enzymes and/or receptors. The enzymes or receptors used in these assays were known to be involved in anticancer activity of known compounds, as well as other diseases, or related to such enzymes or receptors.

### A. Enzymes and Receptors:

5-Lipoxygenase (5-LO) catalyzes the oxidative metabolism of arachidonic acid to 5-hydroxyeicosatetraenoic acid (5-HETE), the initial reaction leading to formation of leukotrienes. Eicosanoids derived from arachidonic acid by the action of lipoxygenases or cycloxygenases have been found to be involved in acute and chronic inflammatory diseases (i.e. asthma, multiple sclerosis, rheumatoid arthritis, ischemia, edema) as well in neurodegeneration (Alzheimer's disease), aging and various steps of carcinogenesis, including tumor promotion, progression and metastasis. The aim of this study was to determine whether Compound 1, is able to block the formation of leukotrienes by inhibiting the enzymatic activity of human 5-LO.

Acyl CoA-Cholesterol Acyltransferase (ACAT) converts cholesterol to cholesteryl esters and is involved in the development of artherioscerosis.

Cyclooxygenase-2 (COX-2) enzyme is made only in response to injury or infection. It produces prostaglandins involved in inflammation and the immune response. Elevated levels of COX-2 in the body have been linked to cancer.

The peripheral benzodiazepine receptor (PBR or PBenzR) is a well-characterized receptor known to be directly involved in diseases states. PBR is involved in the regulation of immune responses. These diseases states include inflammatory diseases (such as rheumatoid arthritis and lupus), parasitic infections and neurodegenerative diseases (such as Alzheimer's, Huntington's and Multiple Sclerosis). This receptor is known to be involved in anticancer activity of known compounds.

Leukotriene, Cysteinyl (CysLT₁) is involved in inflammation and CysLT₁-selective antagonists are used as treatment for bronchial asthma. CysLT₁ and 5-LO were found to be upregulated in colon cancer.

GABA_{A}, the Central Benzodiazepine Receptor (CBenzR or CBR) is involved in anxiolitic activities.

### B. General Procedures:

The procedures used were based on known assays: ACAT (from rat; Ref: Largis et al (1989), J. Lipid. Res., vol 30, 681-689), COX-2 (human; Ref: Riendeau et al (1997), Can. J. Physiol. Pharmacol., vol 75, 1088-1095 and Warner et al (1999), Pro. Natl. Acad. Sci. USA, vol 96, 7563-7568), 5-LO (human; Ref: Carter et al (1991), J. Pharmacol. Exp. Ther., vol 256, no 3, 929-937, and Safayhi et al (2000), Planta Medica, vol 66, 110-113), PBR (from rat; Le Fur et al (1983), Life Sci. USA, vol 33, 449-457), CysLT₁ (human; Martin et al (2001), Biochem. Pharmacol., vol 62, no 9, 1193-1200) and CBR (from rat; Damm et al (1978), Res. Comm. Chem. Pathol. Pharmacol., vol 22, 597-600 and Speth et al (1979), Life Sci., vol 24, 351-357).

### C. Binding Assay of Compound 1 on 5-LO:

Human peripheral blood mononuclear cells (PMNs) were isolated through a Ficoll-Paque density gradient. PMNs were stimulated by addition A23187 (30 μM final concentration). Stimulated PMNs were adjusted to a density of 5×10⁶ cells/mL in HBBS medium and incubated with the vehicle control (DMSO), Compound 1 (at final concentrations of 0.1, 0.5, 1, 2.5, 5 and 10 μM) and NDGA as positive control (at final concentrations of 3,1, 0.3, 0.1 and 0.03 μM) for 15 minutes at 37° C. Following incubation, samples were neutralized with NaOH and centrifuged. Leukotriene B4 content was measured in the supernatant using an Enzyme Immunosorbant Assay (EIA) assay. The experiment was performed in triplicate.

Results shown in FIG. 1 demonstrated that Compound 1 inhibited the activity of human 5-LO with an apparent IC₅₀=0.93 μM (versus 0.1 μM for the positive control NDGA) and therefore displays anti-inflammatory properties.

### D. Percentage Inhibition or Binding of Compounds 1-12 and 46:

Binding assays were done for each of Compounds 1-12 and 46 using ACAT, COX-2, 5-LO, PBR and CysLT₁ enzymes. The procedures used are based on the respective references mentioned above and the conditions are summarized in Tables 16 (enzyme assays) and 17 (radioligand receptor assays).

TABLE 16
Enzyme Assays Conditions

| | Source | Substrate | Pre-I^{a} | I^{b} |
|---|---|---|---|---|
| ACAT^{c} | Wistar rat hepatic | 12.7 μM [¹⁴C]palmitoyl CoA | 5 min/37° C. | 10 min/37° C. |
| | microsomes | | | |
| COX-2^{d} | Human recombinant | 0.3 μM arachidonic acid | 15 min/37° C. | 5 min/37° C. |
| | insect Sf21 cells | | | |
| 5-LO^{e} | Human PBML cells | Arachidonic acid | 15 min/37° C. | 15 min/37° C. |

^{a}Pre-Incubation Time/Temperature
^{b}Incubation Time/Temperature
^{c}Incubation buffer: 0.2 M phosphate buffer (pH 7.4 at 25° C.); Method: Quantitation of [¹⁴C]cholesterol ester by column chromatography.
^{d}Incubation buffer: 100 mM Tris-HCl, pH 7.7, 1 mM glutathione, 1 μM hematin, 500 μM phenol; Method: EIA quantitation of PGE₂.
^{e}Incubation buffer: HBSS (Hank's balanced salt solution); Method: EIA quantitation of LTB₄.

TABLE 17
Radioligand Binding Assays Conditions^{a}

| | | | | Non-spec |
|---|---|---|---|---|
| | Source | Ligand | I^{b} | ligand |
| PBR^{c} | Wistar rat heart | 0.3 nM [³H]PK-11195 | 15 min/25° C. | Dipyridamole^{f} |
| CysLT₁^{d} | Human | 0.3 nM [³H]leukotriene | 30 min/25° C. | Leukotriene D₄^{g} |
| | recombinant CHO- | D₄ | | |
| | K1 cells | | | |
| CBR^{e} | Wistar rat brain | 1 nM [³H]flunitrazepam | 60 min/25° C. | Diazepam^{h} |

^{a}Quantitation Method: Radioligand binding
^{b}Incubation Time/Temperature
^{c}Incubation buffer: 50 mM Tris-HCl, pH 7.5, 10 mM MgCl₂ at 25° C.
^{d}Incubation buffer: 50 mM Tris-HCl, pH 7.4, 5 mM CaCl₂, 5 mM MgCl₂, 100 μg/mL bacitracin, 1 mM benzamidine, 0.1 mM PMSF.
^{e}Incubation buffer: 50 mM Na—K phosphate, pH 7.4 at 25° C.
^{f}Non specific ligand: 100 μM, K_{D}: 2.3 nM, Bₘₐₓ: 0.17 pmol/mg protein, Specific binding: 90%
^{g}Non specific ligand: 0.3 μM, K_{D}: 0.21 nM, Bₘₐₓ: 3 pmol/mg protein, Specific binding: 93%
^{h}Non specific ligand: 10 μM, K_{D}: 4.4 nM, Bₘₐₓ: 1.2 pmol/mg protein, Specific binding: 91%

Binding Assays were done at constant concentration of the compound, in 1% DMSO as vehicle, and are specified below each enzyme/receptor type in Table 18. The results are expressed in Table 18 as percentage inhibition. Significance was obtained when a result was ≧50% binding or inhibition (underlined).

TABLE 18
Percentage of inhibition or binding activity

| | ACAT | COX-2 | 5-LO | PBR | CysLT₁ | CBR |
|---|---|---|---|---|---|---|
| Compound | (10 μM) | (4 μM) | (4 μM) | (1 μM) | (4 μM) | (10 μM) |
| 1 | 90 | 96 | 99 | 80 | 92 | 39 |
| 2 | 51 | 92 | 93 | 65 | 75 | 22 |
| 3 | 63 | 76 | 72 | 11 | 59 | 10 |
| 4 | 65 | 78 | 98 | 92 | 64 | 12 |
| 5 | 60 | 63 | 98 | 68 | 72 | 21 |
| 6 | 54 | 45 | 71 | 75 | 24 | 14 |
| 7 | 95 | 26 | 63 | 65 | 15 | 21 |
| 8 | 40 | 19 | −13 | 55 | 13 | 1 |
| 9 | 77 | 44 | 96 | 32 | 70 | 10 |
| 10 | 90 | 45 | 97 | 86 | 67 | 5 |
| 11 | 71 | 57 | 97 | 39 | 74 | 20 |
| 12 | 83 | 30 | 86 | 39 | 33 | −24 |
| 46 | 8 | 95 | 65 | −1 | 71 | 27 |

All of the exemplified Compounds 1-12 and 46 possessed inhibition and/or binding activity. None of them significantly bound the central benzodiazepine receptor (CBR), which demonstrated that selectivity for the peripheral receptor was present.

PBR binding studies using multiple dilutions indicated that Compound 1 had an inhibition concentration (IC₅₀) value of 0.291 μM and an inhibition constant (Ki) of 0.257 μM, compared to the binding results above, which showed an IC50 above 10 μM in the inhibition of CBR.

Also treatment of animals with Compound 1 resulted in an increased expression of several genes involved in steroid biosynthesis, cholesterol transport/metabolism, signal transduction and apoptosis, which is consistent with Compound 1 acting as a PBR ligand.

### Example 13

### In Vitro Profiling of the Compounds of the Invention

(a) In Vitro Anticancer Activity of the Compounds of Formula I against Four Cell Lines:

In vitro cytotoxic activities of exemplified Compounds are shown in Table 19, along with hemolytic activity of each compound. Compounds were tested in four cell lines: HT-29 (colorectal carcinoma), SF268 (CNS), MDA-MB-231 (mammary gland adenocarcinoma) and PC-3 (prostate adenocarcinoma). Procedures used for each series of tests are described below.

TABLE 19
In vitro Cytotoxic Activities and Hemolysis

| Compound | HT-29 | SF-268 | PC-3 | MDA-MB-231 | Hemolysis^{c} |
|---|---|---|---|---|---|
| No: | (GI₅₀ μM) | (GI₅₀ μM) | (GI₅₀ μM) | (GI₅₀ μM) | (ED₅₀ μg/mL) |
| 1^{a/b} | 11.2/9.33 | 1.96/1.55 | 1.95/3.76 | 1.79/3.18 | 7.6 |
| 2^{b} | 0.65 | 0.12 | 0.45 | 0.24 | 5.12 |
| 3^{a} | 7.3 | 5.73 | 5.36 | 6.32 | >64 |
| 4^{a} | 14.7 | 4.97 | 5.86 | 11.3 | >64 |
| 5^{a} | 14.4 | 13.4 | 15.6 | 20.5 | >64 |
| 6^{a} | >30 | 18.9 | 19.0 | 24.6 | >64 |
| 7^{a} | 14.1 | 18.5 | 14.6 | 17.4 | >64 |
| 9^{a} | 12.6 | 1.88 | 1.44 | 2.48 | >64 |
| 10^{a} | 13.0 | 2.02 | 1.35 | 1.55 | >64 |
| 11^{a} | 16.0 | 5.79 | 5.35 | 7.72 | 9.8 |
| 12^{a} | 9.33 | 1.95 | 1.2 | 2.79 | >64 |
| 14^{b} | 2.04 | 0.76 | 1.15 | 2.16 | 43.9 |
| 17^{b} | >30 | 13.4 | 18.7 | >30 | 35.0 |
| 18^{b} | >30 | 7.45 | >30 | >30 | >64 |
| 46^{a} | 4.26 | 0.72 | 0.90 | 0.59 | 13.9 |
| 63^{b} | 2.57 | 0.89 | 1.25 | 2.27 | >64 |
| 64^{b} | 2.5 | 0.56 | 1.14 | 1.39 | >64 |
| 67^{b} | 2.44 | 0.53 | 1.33 | 1.92 | >64 |
| 77^{b} | 13.9 | 3.31 | 17.1 | 5.62 | 60.9 |
| 78^{b} | 0.29 | 0.07 | 0.23 | 0.24 | 9.89 |
| 80^{b} | 1.43 | 0.33 | 1.80 | 1.02 | >64 |
| 82^{b} | 23.6 | 4.75 | 13.4 | 11.0 | >64 |
| 83^{b} | 19.6 | 9.74 | 13.2 | 6.71 | 12.4 |
| 84^{b} | 21.5 | 3.49 | 16.4 | 23.5 | >32 |
| 85^{b} | 1.89 | 1.73 | 1.08 | 2.19 | >64 |
| 87^{b} | 1.83 | 0.91 | 1.39 | 2.40 | >64 |
| 89^{b} | >30 | 13.7 | 13.5 | 25.3 | >64 |
| 92^{b} | >30 | 13.5 | 16.6 | 11.1 | >64 |
| 97^{b} | 2.02 | 2.04 | 1.19 | 2.02 | 15.1 |
| 98^{b} | 0.69 | 0.16 | 0.82 | 0.51 | 4.5 |

^{a}Results obtained by method (1) below
^{b}Results obtained by method (2) below
^{c}Hemolysis is measured as the concentration necessary to achieve 50% hemolysis of SRBC (Amphotericin B: 4 μg/mL)

### Method (1):

Cytotoxic activities were determined in vitro for Compounds 1, 3-7, 9-12 and 46 to determine the concentration of each compound needed to obtain a 50% inhibition of cell proliferation (GI₅₀). The GI₅₀ value emphasizes the correction for the cell count at time zero and, using the seven absorbance measurements [time zero, (Tz), control growth, (C), and test growth in the presence of drug at the five concentration levels (Ti)], GI₅₀ is calculated as [(Ti−Tz)/(C−Tz)]×100=−50, which is the drug concentration resulting in a 50% reduction in the net DNA increase in control cells during the drug incubation.

Compounds were dissolved at 10 mM in DMSO. Dilution in vehicle to concentrations of 30, 10, 3, 1 and 0.3 μM were prepared immediately before assays. Depending on the cell line's growth characteristics, 4000-10000 cells were plated in two 96-wells pates (day 0) and incubated for 16 hours. The following day, propidium iodide was added to one of the two plates and fluorescence measured (Tz). Test compounds were added to the second plate, as well as vehicle control, and cells further incubated for 96 hours. Each compound was tested at each concentration and in triplicates. The equivalent cell number was determined after adding propidium iodide by measuring the signal by fluorescence (C for control). GI₅₀ results were calculated using the formula above and are shown in Table 19.

Compound 2 has an unexpected increase in cytotoxic activity compared to parent Compound 1. A fifty fold increase of activity was observed against HT-29 cell line. Cytotoxic activity (GI₅₀) of Compound 2 for the other three strains was outside the expected range of concentrations used in the first tests. The second test showed nanomolar activities for Compound 2, a 100-fold increase in potency.

### Method (2):

In vitro cytotoxic activities (GI₅₀) of Compounds 1, 2, 14, 17, 18, 63, 64, 67, 77, 78, 80, 82 to 85, 87, 89, 92 and 96 to 98, were determined using propidium iodide (PI) as being the concentration of drug resulting in 50% growth inhibition, and by using the following method.

Two 96-well plates were seeded in duplicate with each cell line at the appropriate inoculation density (HT29: 3,000; SF268: 3,000; PC-3: 3,000; and MDA-MB-231: 7,500 cells) and according to the technical data sheet of each cell line (rows A-G, 75 μL of media per well). Row H was filled with medium only (150 μL, negative control-medium). The plates were incubated at appropriate temperature and CO₂ concentration for 24 hrs.

Test Compounds were prepared as 15× stock solutions in appropriate medium and corresponding to 450, 45, 0.45, 0.045, and 0.0045 μM (prepared the day of the experiment). An aliquot of each was diluted 7.5-fold in appropriate test medium to give a set of six 2× concentration solutions (60, 6, 0.6, 0.06, 0.006, and 0.0006 μM). A 75 μL aliquot of each concentration was added to each corresponding well (rows A to F) of the second plate. Row G was filled with 75 μL of medium/0.6% DMSO (negative control-cells). The second plate was incubated at appropriate temperature and CO₂ concentration for 96 hrs.

First Plate: Pi (30 μL, 50 μg/mL) was added to each well of the first plate without removing the culture medium. The plate was centrifuged (Sorvall Legend-RT, swinging bucket) at 3500 rpm/10 min. Fluorescence intensity (Thermo, Varioskan, λₑₓ: 530 nm; λₑₘ: 620 nm) was measured to give the first measurement, dead cells (DC at T₀; before freezing). Two round of Freeze (−80° C.)/Thaw (37° C.) were done. Fluorescence intensity was determined to give the second measure, total cells (TC at T₀; after freeze/thaw)

Second plate was processed as the first one, except there were three rounds of freeze/thaw instead of two. First measurement gave the treated dead cells value (TDC), and the second measurement gave the treated total cells value (TTC). Both values were collected for each treated well and control (CTC and CDC).

Each value (DC, TC, TDC, TTC, CTC and CDC) was corrected by removing the background value (medium only) to give the value (FU_{DC(T=0)}, FU_{TC(T=0)}, FU_{TDC}, FU_{TTC}, FU_{CTC} and FU_{CDC}) used in the calculation of the T/C (%) (Treated/Control) for each concentration. T/C (%) for each concentration is calculated using the following formula:

$$T \text{/} C ( \% ) = \frac{( {FU}_{TTC} - {FU}_{TCD} ) - ( {FU}_{TC ( T = 0 )} - {FU}_{D C ( T = 0 )} ) \times 100}{( {FU}_{CTC} - {FU}_{CDC} ) - ( {FU}_{TC ( T = 0 )} - {FU}_{D C ( T = 0 )} )}$$

The GI₅₀ value emphasizes the correction for the cell count at time zero for cell survival. The T/C values are transposed in a graph to determine GI₅₀ values, the concentration at with the T/C is 50%.

(b) Anticancer Activity Profiling of Compound 2 against 36 Cell Lines (IC₅₀):

Culture conditions and activity evaluations were done as indicated for Compound 1 in Example 13 (a) below. Results were expressed as the concentration of drug which inhibits 50% of the cell growth (IC₅₀). The low micromolar to nanomolar levels of IC₅₀ values shown in Table 20 demonstrated a pharmacologically relevant cytotoxic activity of Compound 2 against a variety of 36 tumor types including melanomas, pancreatic, lung, colon, gastric, bladder, renal, CNS, head and neck, prostate, uterus, ovarian and breast carcinomas.

TABLE 20
In vitro profiles of Compound 2 (IC₅₀)

| | | | | Histology in | IC⁵⁰ |
|---|---|---|---|---|---|
| # | Type | Cell line | Origin | nude mice | (μM) |
| 1 | Bladder | T24 | ATCC | transitional cell ca | 0.127 |
| 2 | Bladder | 1218L | Xenograft | urothelial adeno ca | 0.166 |
| 3 | Colon | HCT116 | NCI | adeno ca, pd | 0.156 |
| 4 | Colon | HT29 | NCI | adeno ca, pd | 0.223 |
| 5 | CNS | 498NL | Xenograft | glioblastoma | 0.176 |
| 6 | CNS | SF268 | NCI | nd | 0.010 |
| 7 | Gastric | 251L | Xenograft | adeno ca, pd | 0.105 |
| 8 | Head & Neck | 536L | Xenograft | hypopharynx ca | 0.181 |
| 9 | Lung | 1121L | Xenograft | large cell ca | 0.125 |
| 10 | Lung | 289L | Xenograft | adeno ca | 1.553 |
| 11 | Lung | 526L | Xenograft | adeno ca | 0.104 |
| 12 | Lung | 629L | Xenograft | adeno ca | 0.164 |
| 13 | Lung | 529L | Xenograft | large cell ca, | 0.127 |
| 14 | Lung | H460 | NCI | large cell ca | 0.366 |
| 15 | Mammary | 401NL | Xenograft | pap adeno ca, wd | 0.194 |
| 16 | Mammary | MCF7 | NCI | mamma ca | 0.276 |
| 17 | Melanoma | 276L | Xenograft | mm, amelanotic | 1.948 |
| 18 | Melanoma | 394NL | Xenograft | mm, amelanotic, pd | 0.020 |
| 19 | Melanoma | 462NL | Xenograft | mm, amelanotic | 0.978 |
| 20 | Melanoma | 514L | Xenograft | mm, melanotic | 0.110 |
| 21 | Melanoma | 520L | Xenograft | mm, melanotic | 0.085 |
| 22 | Ovarian | 1619L | Xenograft | adeno ca, md | 0.579 |
| 23 | Ovarian | 899L | Xenograft | pap serous ca, md | 0.238 |
| 24 | Ovarian | OVCAR3 | NCI | adeno ca, md | 0.139 |
| 25 | Pancreas | 1657L | Xenograft | adeno ca, md | 1.777 |
| 26 | Pancreas | PANC1 | ATCC | nd | 0.125 |
| 27 | Prostate | 22RV1 | ATCC | adeno ca, md | 0.142 |
| 28 | Prostate | DU145 | NCI | adeno ca, md | 0.158 |
| 29 | Prostate | LNCAP | DSMZ | adeno ca, md | 0.485 |
| 30 | Prostate | PC3M | NCI | adeno ca, pd | 0.114 |
| 31 | Pleuramesot. | 1752L | Xenograft | pleuromesothelioma | 1.503 |
| 32 | Renal | 1781L | Xenograft | renal ca | 0.172 |
| 33 | Renal | 393NL | Xenograft | hypernephroma, wd | 0.527 |
| 34 | Renal | 486L | Xenograft | hypernephroma, pd | 1.144 |
| 35 | Renal | 944L | Xenograft | hypernephroma | 0.230 |
| 36 | Uterus | 1138L | Xenograft | carcinosarcoma, wd | 0.139 |
| | | | | Mean of all | 0.407 |
| | | | | cell lines: | |

ca = carcimoma;
pd = poorly differentiated;
pap = papillary;
md = moderately differentiated;
wd = well differentiated;
mm = malignant melanoma;
nd = not determined

### Example 14

### Pharmacokinetic Profiles

Compounds 1 and 2 were separately dissolved in ethanol (5%), Polysorbate 80 (15%), PEG 400 (5%) and dextrose (5%) at a final concentration of 6 mg/ml. Prior to dosing, animals (female Crl: CD1 mice; 6 weeks of age, 22-24 g) were weighed, randomly selected and assigned to the different treatment groups. Compound 1 and Compound 2 were administered by the intravenous (IV) or intraperitoneal (IP) route to the assigned animals. The dosing volume of Compounds 1 and 2 was 5 mL per kg body weight. Animals were anesthetized with 5% isoflurane prior to bleeding. Blood was collected into microtainer tubes containing the anticoagulant K₂EDTA by cardiac puncture from each of 4 animals per bleeding timepoint (2 min, 5 min, 15 min, 30 min, 1 h, 2 h, 4 h and 8 h). Following collection, the samples were centrifuged and the plasma obtained from each sample was recovered and stored frozen (at approximately −80° C.) pending analysis. Samples were analysed by LC/MS/MS. Standard curve ranged from 25 to 2000 ng/mL with limit of quantitation (LOQ)<25 ng/mL and limit of detection (LOD) of 10 ng/mL.

Plasma values of Compounds 1 and 2 falling below the limit of quantitation (LOQ) were set to zero. Mean concentration values and standard deviation (SD) were calculated at each timepoints of the pharmacokinetic study (n=4 animals/timepoint). The following pharmacokinetic parameters were calculated: area under the plasma concentration versus time curve from time zero to the last measurable concentration time point (AUC₀₋ₜ), area under the plasma concentration versus time curve extrapolated to infinity (AUC_{inf}), maximum observed plasma concentration (Cₘₐₓ), time of maximum plasma concentration (tₘₐₓ), apparent first-order terminal elimination rate constant (kₑₗ), apparent first-order terminal elimination half-life will be calculated as 0.693/kel (t₁₂). The systemic clearance (CL) of Compound 1 after intravenous administration was calculated using Dose/AUCinf. Pharmacokinetic parameters were calculated using Kinetica™ 4.1.1 (InnaPhase Corporation, Philadelphia, Pa.).

### Results:

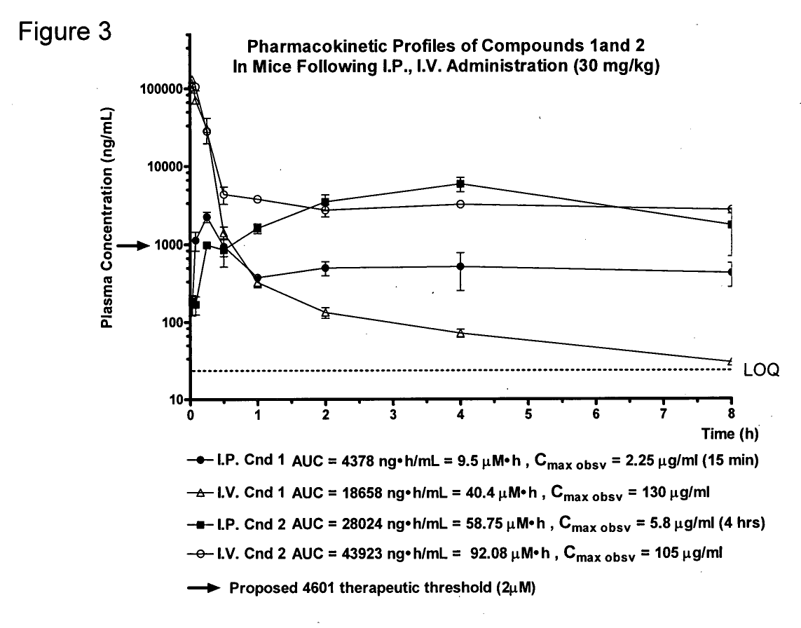

Mean plasma concentrations of Compound 2 following iv and ip administrations at 30 mg/kg, compared with Compound 1 via the same routes of administration, are presented in FIG. 3. When administered iv, Compound 2 had an AUC of 92.08 μM·h and an observed Cₘₐₓ of 105 μg/mL, compared to an AUC of 40.4 μM·h and an observed Cₘₐₓ of 130 μg/mL for Compound 1. When administered IP, Compound 2 had an AUC of 58.75 μM·h and an observed Cₘₐₓ of 5.8 μg/mL, compared to an AUC of 9.5 μM·h and an observed Cₘₐₓ of 2.25 μg/mL for Compound 1. Mean (±SD) plasma concentrations of Compound 1 following IV administration of a 30 mg/kg dose declined rapidly in a biexponential manner resulting in very short half lives (t_{1/2} α and β of 4.6 min and 2.56 h, respectively). The pharmacokinetics of Compound 1 following intraperitoneal administration, and Compound 2 following intraperitoneal and intravenous administration, showed a PK profile suggestive of slow release. With these routes of administration, the compound plasma concentration was sustained and maintained at therapeutically relevant levels for over 8 hours. Compound 2 showed a half life (t_{1/2}) of more than 40 hours following both IP and IV administrations.

Acute toxicity studies in CD-1 nu/nu mice for Compound 2, using the same formulation, gave an MTD ≧50 mg/kg (ip, NOAEL: 30 mg/kg) and >100 mg/kg (iv, NOAEL: 75 mg/kg), with weight losses of about 7% for several days post-injection. Compound 1 had an MTD of 150 mg/kg when administered iv. Acute toxicity studies with Compound 46 gave an MTD of 30 mg/kg (ip).

### Example 15

### In Vitro Anticancer Activity of Compound 1

a) Human Tumor Cell Lines from the U.S. NCI Panel

A study measuring the in vitro cytotoxic activity of Compound 1 was first performed by the NCI (National Cancer Institute, U.S. National Institutes of Health, Bethesda, Md., USA) against a panel of human cancer cell lines. This screen utilizes 60 different human tumor cell lines, representing cancers of the blood, skin, lung, colon, brain, ovary, breast, prostate, and kidney (More information available at: http://dtp.nci.nih.gov/branches/btb/ivclsp.html.) The compound was sent and tested on three occasions (Mar. 31, 2003; Dec. 1, 2003; Mar. 27, 2007).

The results from the NCI in vitro screening indicate that Compound 1 has broad cytotoxic activity in the low micromolar range in the 60 different cell lines tested. The compound showed activity in vitro against leukemia (GI₅₀ range=0.9-5.0 μM), non-small cell lung carcinoma (GI₅₀ range=1.9-10.8 μM), melanoma (GI₅₀ range=1.8-8.1 μM), prostate carcinoma (GI₅₀ of 3.5-9.3 μM), breast carcinoma (GI₅₀ range=1.4-16.3 μM), ovarian carcinoma (GI₅₀ range=2.5-6.2 μM), renal carcinoma (GI₅₀ range=2.9-14.5 μM), colon carcinoma (GI₅₀ range=3.0-17.3 μM) and CNS (glioblastoma, GI₅₀ range=2.0-6.5 μM) tumor cell lines.

Following the “flat” pattern of activity of Compound 1 across the cell lines tested, no significant correlation was observed using the COMPARE alogorithm.

### b) Human and Animal Glioma Cell Lines (IC₅₀)

The cytotoxic activity of Compound 1 was further evaluated using a panel of brain tumor cell lines. This study was performed in collaboration with INSERM (Grenoble, France). Tumor cells (5,000 to 10,000 cells per well depending on their doubling time) were plated in 96-well flat-bottom plates and incubated for 24 hours before treatment. Tumor cells were then incubated for 96 hours with seven different concentrations of Compound 1: 10, 1, 0.5, 0.1, 0.5, 0.01, and 0.001 μM. The in vitro cytotoxic activity was determined by a standard MTT assay. Results in Table 21 are expressed as the concentration of drug that inhibits 50% of the cell growth (IC₅₀) as compared to non-treated control cells.

TABLE 21

| | | | IC₅₀ at 96 h |
|---|---|---|---|
| Cell line | Type | Origin | (μM) |
| 9L | Gliosarcoma | Rat | 8.3 ± 3.8 (n = 4) |
| GHD | Astrocytoma | Human | 6.5 ± 2.9 (n = 8) |
| U 373 | Astrocytoma | Human | 3.8 ± 1.4 (n = 4) |
| GL26 | Glioblastoma | Human | 8.9 ± 1.1 (n = 4) |
| C6 | Glioblastoma | Rat | 4.3 ± 2.3 (n = 5) |
| DN | Oligodendroglioma | Human | 3.0 ± 0.7 (n = 4) |
| GHA | Oligodendroglioma | Human | 1.6 ± 0.7 (n = 10) |

The IC₅₀ values of Compound 1 against different representative types of brain tumor cell lines were similar, ranging from 1.6 to 8.9 μM. These results confirmed the activity of ECO-4601 against different brain cancer cell lines including a rat glioblastoma C6 cell line, which is the most malignant form of brain cancer, type IV glioblastoma multiforme.

### Example 16

### Benzodiazepine Receptor Binding Assays

As Compound 1 was isolated from structural prediction through genetic analysis and activity identified through in vitro cytotoxic assays, its molecular target(s) were unknown at the time of discovery. Based on the structural characteristics of ECO-4601, we first investigated its binding affinity to the central (GABA_{A}; CBR;) and peripheral (PBR) benzodiazepine receptors. The effect of ECO-4601 on CBR (GABA_{A}) and PBR was initially evaluated in a radioligand-binding assay at MDS Pharma Services (Taipei, Tawain). CBR and PBR were obtained from rat brain and heart membrane-fractions, respectively. Displacement assays were done in the presence of 1 nM [³H]-Flunitrazepam (CBR; GABA_{A}) or 0.3 nM of [³H]-PK11195 (PBR). ECO-4601 was tested at 0.01, 0.1, 0.5, 1, 5 and 10 μM. Non-specific binding was estimated in the presence of 10 μM diazepam (CBR) or 100 μM dipyrimadole (PBR) and assays were performed according to previous described methods (Damm et al Res Commun Chem Pathol Pharmacol 22, pp 597-600; Le Fur et al (1983) Life Science 33, pp 449-57). Results obtained from these binding studies indicated that ECO-4601 did not bind the CBR (IC₅₀>10 μM) while the binding affinity for the PBR was ˜0.3 μM. The binding affinity of ECO-4601 to the PBR is similar to the concentration required to inhibit cell proliferation (1 to 10 μM, depending on cell lines). This contrats with current specific PBR ligands, which bind the PBR with nanomolar affinity yet their effect on cell proliferation, is in the micromolar range.

### a) Establishment of a PBR Binding Assay:

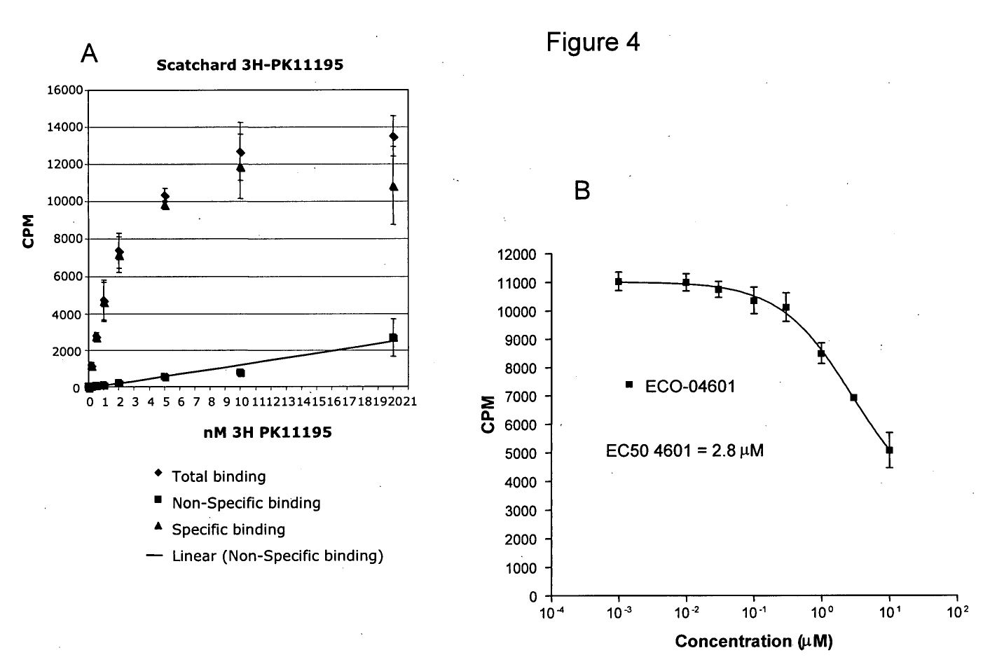

In order to screen analogs of Compound 1 for PBR binding affinity, a PBR binding assay was implemented at Thallion Pharmaceuticals. Hearts obtained from 3 Sprague Dawley rats were homogenized in 20 volumes of ice-cold 50 mM Tris-HCl, pH 7.5. After two centrifugations at 1500 g for 10 minutes at 4° C., the supernatant was centrifuged at 48000 g for 20 minutes at 4° C. The resulting pellet was resuspended in 50 mM Tris-HCl pH 7.5 and protein concentration was estimated by the Bradford colorimetric staining method using BSA as the standard. For equilibrium binding parameters determination, [³H]PK11195 (specific activity, 84.8 Ci/mmol) binding assays were conducted in a final volume of 300 μl of PBR-binding buffer (50 mM Tris-HCl, pH 7.5 and 10 mM MgCl₂) containing the enriched mitochondria membrane preparation (25 μg of protein) and 0.2 nM to 20 nM of [³H]PK11195. In parallel, non-specific binding was measured with the presence of 20 μM cold PK11195. Samples were distributed onto 96-well GF/B filtration plates and incubated for 60 minutes at 25° C. and then washed once with PBR-binding buffer. Filters were punched out and radioactivity measured on a Perkin Elmer TriCarb 2800 Scintillation counter (Janssen et al (1999) J Pharmaceutical and Biomedical Analysis 20, pp 753-761). Scatchard plot analysis of the data by the GraphPad Prism 3.0 software determined a Kd of 1.37 nM for [3H]PK11195 (FIG. 4A).

### b) PBR Binding Affinity of Compound 1:

Binding affinity of ECO-4601 for the PBR was evaluated using the experimental conditions above. For this assay, 25 μg of enriched mitochondrial membrane fraction was incubated with a fixed concentration of [3H]PK11195 (0.5 nM; specific activity 84.8 Ci/mmol) and increasing concentrations of ECO-4601 (0.01 μM t0 10 μM). From the results presented in FIG. 4B, an EC50 of ECO-4601 was determined by the GraphPad Prism 3.0 software to be 2.8 μM, leading to a calculated a Ki value of about 1.4 μM (using the formula of Kᵢ=EC50/(1+[ligand]/Kd), where the [ligand]=1.6 nM).

c) ECO-4601 Concentrations in Tumors and Brains Obtained from Rat C6 Orthotopic Brain Tumors:

i) Cell Culture and Spheroid Preparation

Rat C6 glioma cells were purchased from the American Type Culture Collection (Manessa, Va.) and grown in Dulbecco's modified Eagle's medium (DMEM) supplemented with 10% FBS, 125 U/mL penicillin G, 125 μg/mL streptomycin sulfate, and 2.2 μg/mL amphotericin B (Fungizone). All culture reagents were obtained from Gibco BRL (Invitrogen Burlington, ON, Canada). Cultures were grown in monolayers and maintained at 37° C. in a humidified atmosphere of 5% CO₂. Upon reaching confluency, spheroids were prepared using the hanging drop method previously described by Del Duca et al. ((2004) J. Neurooncol 67, p 295). Briefly, 20 μl drops of DMEM containing 15,000 cells each were suspended from the lids of culture dishes and the resulting aggregates were transferred to culture dishes base-coated with agar after 72 hours. The resulting spheroids were adequate for in vivo implantation after 48 hours of incubation on agar.

ii) Surgical Implantation of Rat C6 Tumor Cells

Male, Sprague-Dawley rats (250-300 g) (Charles River Canada, St Constant, QC) were anesthetized with 50 mg/kg ketamine and 10 mg/kg xylazine. The right cortical surface in the parietal-occipital region was exposed by craniectomy using a high-powered drill (DREMEL, USA) and the underlying dura and its vessels were carefully removed under a surgical microscope. A piece of the cortex was removed to expose the underlying white matter and a single speroid containing rat C6 tumor cells was placed into the surgical defect. The craniectomy was covered with bone wax (Ethicon, Peterborough, Canada) and the overlying skin sutured. Following recovery from anesthesia, the animals were fed and had access to water ad libitum.

iii) Preparation of Tissue Specimens

Following completion of in vivo studies, animals were sacrificed by anesthetic overdose and decapitated. Brain, tumor, and liver were snap-frozen in liquid nitrogen and stored frozen (−70° C.±10° C.). For blood samples, each blood sample was collected into a K2-EDTA tube and kept on wet ice for a maximum of 30 minutes. Blood samples were centrifuged under refrigeration (2 to 8° C.) for 10 minutes at 1,500 g (RCF). A volume of 25 μL of aqueous 4% w/v L-ascorbic acid was added to a volume of 225 μL of rat plasma in a clean tube, and the samples were thoroughly mixed by inversion. A volume of 125 uL of the resulting mixture was transferred to a separate tube for bioanalysis, while the remaining mixture was maintained as a backup, and both the bioanalysis and back-up portions were frozen on dry ice and stored frozen (−70° C.±10° C.).

iv) Sample Extractions and HPLC/MS/MS Analysis

Rat plasma (50 μL) was mixed with 500 μL of acetone containing 100 ng/mL of the internal standard (Compound 2). The mixture was vortexed for 20 seconds, incubated 10 min at RT and centrifuged at 12,000×g for 10 minutes. The supernatants were transferred into HPLC injection vials and 20 μL were analyzed by tandem liquid chromatography/mass spectrometry. The chromatography was achieved on a Luna C18 column (30×4.6 mm, 5 μm particles; Phenomenex, Torrance, Calif.) with a mobile phase consisting of 100% methanol: 0.5% formic acid in water (85:15) at a flow rate of 1 mL/min. Flow was split 1:10 prior to introduction into the ESI source. The nebulization was assisted by and octagonal jet stream of nitrogen heated at 3500 C and set at a flow of 4 L/min. The ion source voltage (ISV) was at 4000 V in positive mode, declustering potential (OR-QO) was set to 20 V, and the collision energy (Elab) was set to 25 V. The mass transition monitered for ECO-4601 and the internal standard were m/z 463→271 and 477→273, respectively. Standard curve in mouse plasma ranged from 25 to 10,000 ng/mL, with seven calibration points. Table 22 summarizes the levels of Compound 1 (ECO-4601) found in plasma and selected tissues.

TABLE 22

| Pasma | Liver | Brain | Tumor | Ratio |
|---|---|---|---|---|
| ng/mL | ng/g | ng/g | ng/g | Tumor/Brain |
| 13,088 | 994 | 249 | 2701 | 10.1 |
| 2,657 | 164 | 115 | 239 | 2.1 |
| 5,727 | 1083 | 98 | 509 | 5.2 |
| 10,212 | 2767 | 254 | 2891 | 11.30 |
| 1,177 | 123 | 327 | 1145 | 3.5 |

These data demonstrated that ECO-4601 accumulated preferentially in the tumor compared to the brain in a rat orthotopic brain tumor model. Indded, we observed 2 to 10 fold higher levels of Compound 1 in tumor of the brain compared to the rest of the normal brain.

d) In vivo PBR occupancy by ECO-4601

i) Radiosynthesis of ¹¹C-(R)-PK11195

(R)-1-(2-chlorophenyl)-N-methyl-N-(1-methyl-propyl)-3-isoquinoline carboxamide (R N-desmethyl PK11195), the precursor for the radioisotope-labeled (R) —PK11195, was purchased from ABX (Radeburg, Germany). The synthesis of ¹¹C-(R)-PK11195 was accomplished by a modification of the method of Camsonne et al. (J. Label. Compd. Radiopharm., 21: 985-991,1984). Briefly, ¹¹C-methyl iodide (¹¹C—CH₃I) was transferred by a stream of nitrogen gas at a flow rate of 10 ml/min into a reaction medium containing 1 mg or R—N-desmethyl PK11195 previously dissolved in 0.5 mL of dimethylsulfoxide (DMSO) and about 20 mg of sodium hydroxide. The reaction was initiated at room temperature, with a bubbling of the ¹¹C—CH₃I being continued over a 5 minute period. After bubbling was complete, the reaction mixture was left standing for 5 minutes. Purification was performed by high pressure liquid chromatography (HPLC) using a reverse-phase C18 column and a methanol:ammonium formate (20 mM) (80:20) mixture and a mobile phase at a flow rate of 3 mL/min. ¹¹C—(R) —PK11195 was eluted at 11 minutes and obtained with a specific radioactivity of 1-1.5 Ci/mol.

ii) Following IP Administration of ECO-4601

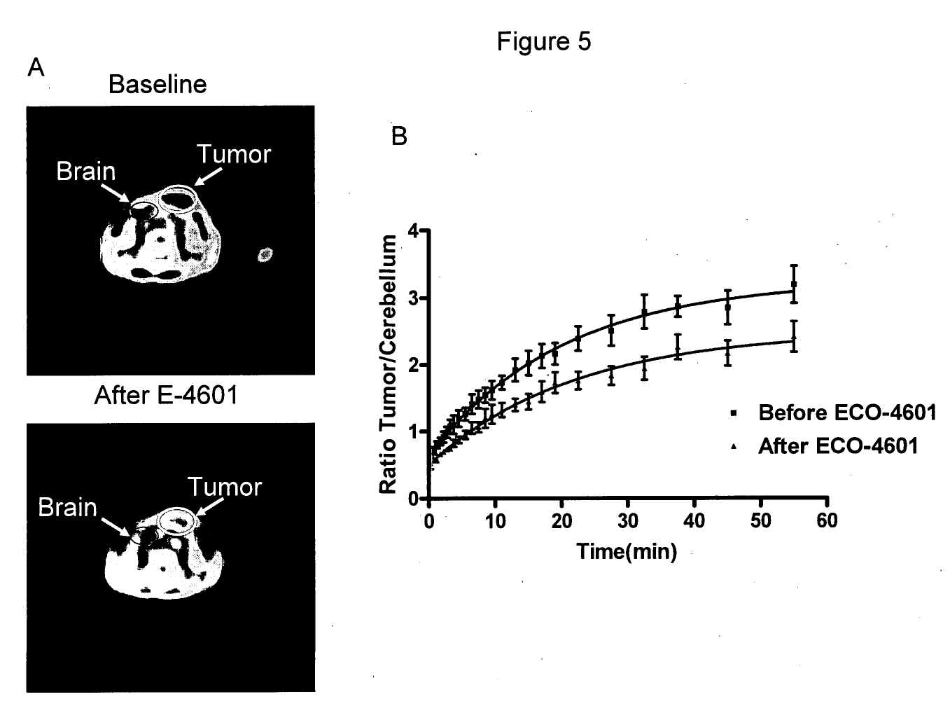

In vivo PET studies were performed 14 days post tumor implantation. Tumor cell growth and tumor implantation was done as described in the above section. Animals were anesthetised with 2% isofluorane and PET imaging studies were performed while the animal was anesthetised using a CTI Concorde R4 microPET scanner (Siemens/CTI Concorde, Knoxville, Tenn.). Competitive binding studies were performed by acquisition of 11C-PK11195 images prior to and 30 min following IP injection of 30 mg/kg of ECO-4601 (FIG. 5A). Each dynamic PET study lasted 60 min and was initiated with an IV bolus administration of 11C-PK11195 (7.1-12.7 MBq) radioligand via the tail vein. Attenuation correction factors, for each rat (total of 7 animals), were determined using a 10 min ⁵⁷Co transmission scan acquired immediately prior to the dynamic scan. In addition, all images were corrected for scattering.

Images were reconstructed using filtered back-projection, and time-activity curves (TACs) were obtained from regions-of-interest (RIOs) in the tumor (target region), brain (reference region), and cerebellum (reference region). For all studies, the mean binding potential (B.P.) was determined using the simplified reference tissue method. Repeated measures t-test analysis was preformed using the R statistics software. P-values less than or equal to 0.05 were considered statistically significant.

Using the simplified reference tissue method, the mean tumor B.P. (baseline)=1.54±0.44 (mean±S.D.) and the mean B.P. (Compound 1)=1.05±0.34 (mean±S.D.). FIG. 5B shows the results of the competitive binding studies during the scanning period. After a dose of 30 mg/kg E-500 IP, the PBR occupancy for ¹¹C-(R)-PK11195 binding was reduced by an average of 31% (P=0.005).

iii) Following IV Administration of ECO-4601

The above study was repeated but his time ECO-4601 was administered as a bolus push followed by a continuous intra-venous (CIV) infusion of ECO-4601 obtained in the steady-state condition through the second 11C-PK11195 PET scan. Seven (7) rats were utilized for this second microPET study

14 days post-tumor implantation, rats received a bolus IV administration of compound 1 (30 mg/kg; 1.5 mL at 0.5 mL per min) followed by continuous IV infusion (5 mg/kg/hour; 0.7 mL/hour) over 3 hours. The animals were then sacrificed by anesthetic overdose and blood was collected by heart puncture. Brain, tumor and liver were snap-frozen in liquid nitrogen and stored at −80° C. until analysis. Blood samples was collected into K₂-EDTA tubes and kept on ice for a maximum of 30 min. Samples were then centrifuged at 4° C. and 1500 g (RCF). A volume of 225 μL of rat plasma was added to clean tubes containing 25 μL of aqueous 4% w/v L-ascorbic acid and samples were mixed and stored at −80° C. until analysis.

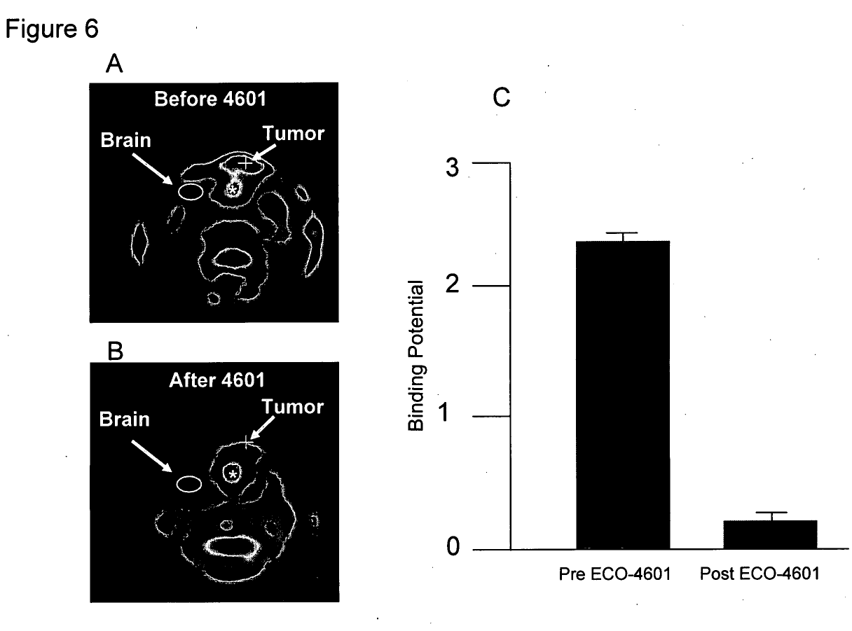

Representative ¹¹C-(R)-PK11195 microPET images from the CIV study are shown in FIGS. 6A and B, which on a comparison of the image presented in FIG. 6B (after administration of ECO-4601) to the image presented in FIG. 6A (before administration of ECO-4601) shows a significant blocking of the radiotracer from the peripheral part of the tumor (area of specific binding) following CIV administration of ECO-4601. An area of non-specific binding is indicated by an asterisk (*) and was considered as a likely necrotic area.

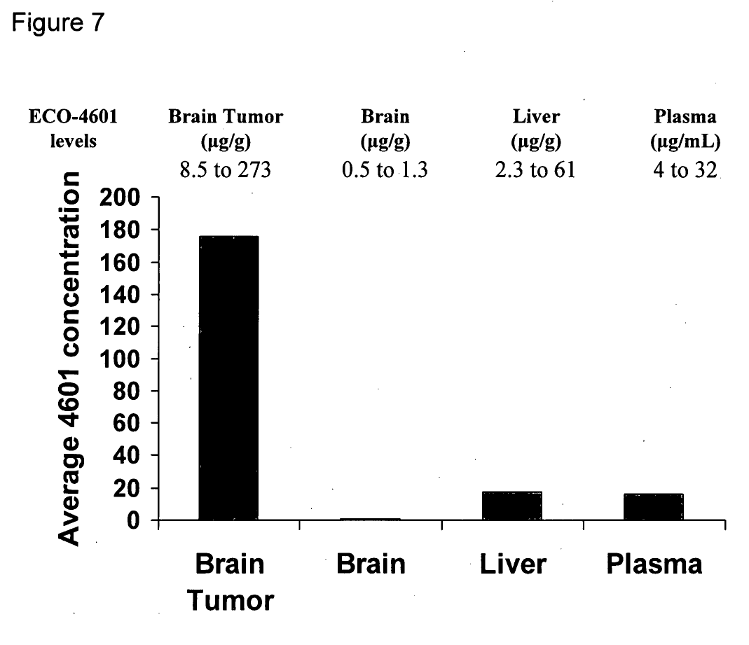

Binding potential (B.P.) data and Compound 1 plasma levels and tissue levels for each of the seven rats included in the study are presented in Table 23 below and summarized in FIG. 7.

TABLE 23

| | | | | Tumor | Brain | Liver | Plasma |
|---|---|---|---|---|---|---|---|
| | Binding | Binding | | Drug | Drug | Drug | Drug |
| | Potential | Potential | % PBR | Level | Level | Level | Level |
| Rat | (Baseline) | (ECO-4601) | occupancy | (μg/g) | (ng/g) | (μg/g) | (ng/mL) |
| A0513 | 2.30 | 0 | 100 | 242 | 941 | 14.2 | 11500 |
| A0519 | 2.59 | 1.81 | 30 | 8.98 | 503 | 3.31 | 4200 |
| A0530 | 1.68 | 0.84 | 50 | 119 | 596 | 2.29 | 4100 |
| A0535 | 2.89 | 0 | 100 | 234 | 527 | 11.2 | 16000 |
| A0536 | 2.51 | 0 | 100 | 8.46 | 692 | 6.91 | 12000 |
| A0539 | 2.11 | 0 | 100 | 182 | 823 | 53.1 | 22000 |
| A0541 | 2.51 | 0 | 100 | 273 | 1265 | 61.0 | 32000 |

As observable from Table 23, the tumor drug level data is relatively consistent between the study animals, with the exception of rats A0519 and A0536. Regarding animal A0519, the low receptor occupancy rate was considered to be due a logistical problem whereby ECO-4601 was not infused for 1 hour between the IV bolus and the start of the PET scan, and therefore the data from this animal was not included in the final analysis (resulting in n=6 animals). Low tumor drug level for animal A0536 was considered to have likely resulted from a compromised tumor vascular supply, although the relatively low final drug level for this animal did not appear to have an impact upon the PBR occupancy level.

To determine a mean tumor binding potential (B.P.) (baseline) and the mean B.P., the simplified reference tissue method was utilized comparing the ratio of tumor to normal brain. As a result, mean tumor binding potential (B.P.) (baseline) was calculated to be 2.19±0.16 (mean±SEM) and the mean B.P. (ECO-4601) was calculated to be 0.14±0.13 (mean±SEM). Graphically, results from the mean B.P calculations from the competition binding studies are shown in FIG. 6C, where it can be observed that after the CIV infusion of ECO-4601, the PBR occupancy for ¹¹C-(R)-PK11195 binding was reduced by an average of 91.67% (P<0.001, n=6).

The studies presented in EXAMPLE 16 clearly demonstrate that Compound 1 binds the PBR both in vitro and in vivo. Furthermore, this binding affinity results in preferential accumulation of Compound 1 in tumor tissue compared to normal tissue as demonstrated by the 10 to 200 fold higher levels of Compound 1 observed in orthotopic rat brain tumors compared with the rest of the brain area (normal tissue). Compound 1 accumulation in the tumor (176 μg/ml) was also significant compared to liver (24.8 μg/g; 7-fold) and plasma (16.2 μg/g; 11-fold) (FIG. 7).

### Example 17

### Effect of Compound 1 on the RAS-MAPK Pathway

Related to its farnesylated moiety, the effect of ECO-4601 was assessed on the Ras signaling pathway. The Ras-MAPK signaling pathway has long been viewed as an attractive pathway for anticancer therapies, based on its central role in regulating the growth and survival of cells from a broad spectrum of human tumors (Downward 2003 Nature Reviews Cancer, 3:11-22; Sebolt-Leopoldd and Herrera 2004 Nature Reviews Cancer 4: 937-947).

It was first verified if ECO-4601 interferes with Ras processing by monitoring FTase and GGTase I activities by immunoblot analysis. For these experiments, exponentially growing MCF-7 (human breast tumor) cells were seeded in 60 mm cell culture dishes at a density of 2.5×10⁶ cells per dish. After an 18 h incubation period, ECO-4601 or lovastatin (Sigma-Aldrich, St. Louis, Mo.) were added at a final concentration of 3, 10 and 30 μM for 24 h. Cells were also treated with the vehicle alone (0.05% DMSO) for the same incubation periods. Treated and control cells were lysed in ice-cold RIPA buffer (50 mM Tris-HCl pH 7.4, 1% Triton X-100, 1% deoxycholate, 0.1% SDS, 150 mM NaCl, and 1 mM EDTA) containing protease inhibitors (Roche, Mannheim, Germany) and phosphatase inhibitors (EMD Biosciences, Calbiochem, San Diego, Calif.) for 20 minutes on ice. After lysis, cell debris was removed by centrifugation at 12000×g for 10 min. Equal amount of protein (30 μg) were separated by SDS-PAGE 8% for HDJ2 or 4-20% gradiant gels for Ras and Rap1A and transferred onto nitrocellulose membranes (0.2 μm; Bio-Rad Laboratories, Hercules, Calif.). Membranes were blocked with 5% skim milk in 1×TBS (Tris-buffered saline) containing 0.1% Tween-20 (TTBS) for 1 h at room temperature, and incubated overnight at 4° C. with the following primary antibodies (5% skim milk-TTBS): RAP1A (Santa Cruz Biotechnology Inc., Santa Cruz, Calif.), HDJ2 (GeneTex Inc., San Antonio, Tex.), Pan-Ras (Calbiochem, EMD Biosciences, San Diego, Calif.) and GAPDH (Santa Cruz Biotechnology Inc.). Bound antibodies were detected using horseradish peroxidase-conjugated secondary antibodies (Jackson ImmunoResearch Laboratories Inc., West Grove, Pa.) for 1 h at room temperature in 0.5% skim milk-TBS, visualized by treating the membranes with enhanced chemiluminescence reagents (Millipore, Mississauga, ON, Canada) and exposing them to BioMax Light Films (Kodak, Mississauga, ON, Canada).

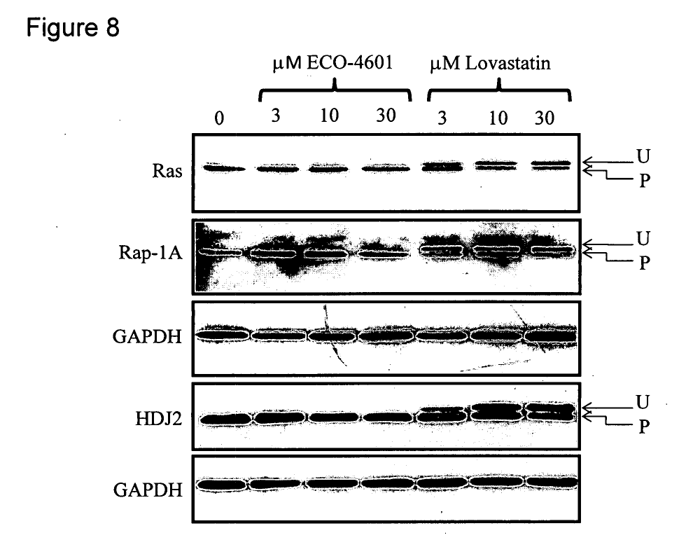

To determine if ECO-4601 is an inhibitor of FTase and/or GGTase 1 activity, an immunoblot method was used to monitor prenylation inhibition of HDJ2, a protein prenylated exclusively by Ftase and RAPLA, a protein prenylated exclusively by GGTase-1. Unprenylated and prenylated proteins can be distinguished by virtue of their different electrophoretic mobilities, the unprenylated forms of HDJ2 and RAP1A displaying a reduced mobility in SDS-PAGE relative to their prenylated forms. MCF7 cells were treated for 24 h with semi-log increasing concentrations of ECO-4601 or lovostatin (a non-specific inhibitor of protein prenylation). The data, presented in FIG. 8, clearly show that while lovostatin treatment resulted in the expected mobility shift of either Ras, RAP1A and HDJ2, ECO-4601 had no effect. These results clearly show that ECO-4601 is not a Farnesyl transferase inhibitor.

The effect of ECO-4601 on downstream events of Ras signaling was subsequently examined by monitoring the phosphorylation levels of Raf-1 and ERK1/2 by Western blot analysis. To study the effect of ECO-4601 on the Ras-MAPK signaling pathway, exponentially growing cells (human breast MCF-7 tumor cells, human breast MDA-MB-231 tumor cells, human glioma U 87-MG tumor cells and human prostate PC-3 tumor cells) were seeded onto 60 mm tissue culture dishes (0.5 to 0.8×10⁶ cells per dish) for 24 h. The media was removed and cells were treated with 10 μM ECO-4601 in culture medium supplemented with 0.1% FBS for 30 min, 1 h, 4 h and 6 h, and subsequently exposed to EGF at 50 ng/mL for 10 min at 37° C. Control plates consisted of cells incubated in culture medium containing 0.1% FBS and 0.05% DMSO (vehicle) with or without EGF stimulation. At the end of each treatment, media was removed and cells rinsed with ice-cold PBS. Cells were then harvested by scraping and cell pellets were lysed in ice-cold RIPA buffer for 20 minutes on ice. Unsolubilized material was pelleted and discarded. The protein concentration of each lysate was quantified using the Bio-Rad protein assay (Bio-Rad Laboratories). Equivalent amounts of protein (20-30 μg protein) were separated on 10% or 12% SDS-PAGE under reducing conditions, transferred onto nitrocellulose membranes (0.2 μm; Bio-Rad Laboratories) and blotted as above with phospho-c-Raf (Ser338) and c-Raf (Cell Signaling Technology Inc., Boston, Mass.), phospho-p44/42 (Thr2O₂/Tyr204, p-ERK1/2) and p44/42 (ERK1/2) MAP Kinases (Cell Signaling Technology Inc.) and GAPDH (SantaCruz Biotechnology Inc.).

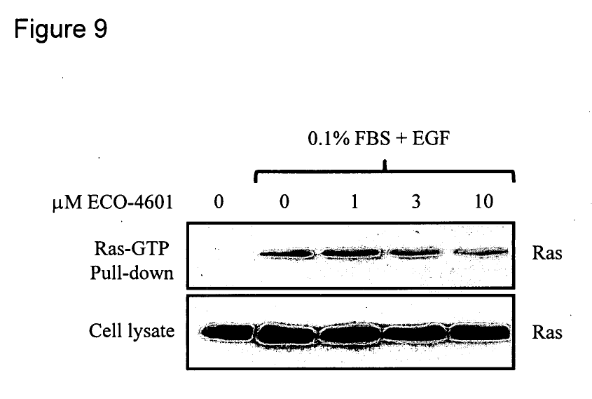

A strong inhibition of EGF-induced phosphorylation of Raf-1 and ERK1/2 was observed (FIGS. 9A and 9B). This effect was time dependent with complete inhibition of protein phosphorylation within 6 h. It was also noted that ECO-4601 not only inhibited Raf-1 phosphorylation, but also caused a decrease in the amount of total Raf-1. The inhibitory activity of ECO-4601 on the Ras-MAPK signaling pathway is thus prior to Raf-1 phosphorylation/degradation and post Ras prenylation.

Unlike current Ras signalling pathway inhibitors, ECO-4601 is not a direct kinase inhibitor. This was documented by evaluating the effect of ECO-4601 on human EGFR, c-RAF, MEK1, MAPK1 (ERK1) and MAPK2 (ERK2) kinase-activity (Upstate Kinase Profiler™ Service; Dundee, UK). ECO-4601 was tested at 0.5 μM and 5 μM in a final volume of 25 μL according to standard protocols developed by Upstate Ltd. Briefly, purified recombinant human enzymes were incubated with 25 mM Tris pH 7.5 containing EGTA, a specific substrate and γ-³²P-ATP. The reaction was initiated with MgATP mix and incubated for 40 minutes at RT. The reaction was stopped by the addition of 5 μL of a 3% phosphoric acid solution; aliquots were spotted on filters and counted. Detailed procedures are available on the Millipore Upstate website. Results of the direct inhibition of kinase activities by ECO-4601, summarized in Table 24, indicate that ECO-4601 does not directly inhibit EGFR, c-Raf, MEK1, ERK1 or ERK2 kinase activities.

TABLE 24

Kinase Activity* (%) ± SD

| | ECO-4601 | ECO-4601 |
|---|---|---|
| Kinases | (0.5 μM) | (5 μM) |
| EGFR | 128 ± 6 | 127 ± 9 |
| c-Raf | 114 ± 12 | 94 ± 2 |
| MEK1 | 106 ± 2 | 98 ± 1 |
| MAPK1 (ERK1) | 97 ± 2 | 73 ± 3 |
| MAPK2 (ERK2) | 116 ± 1 | 110 ± 1 |

*Data is expressed as the percentage of enzyme activity in the presence of ECO-4601 over that of the positive control. Results are the mean of 2 separate experiments ± SD.

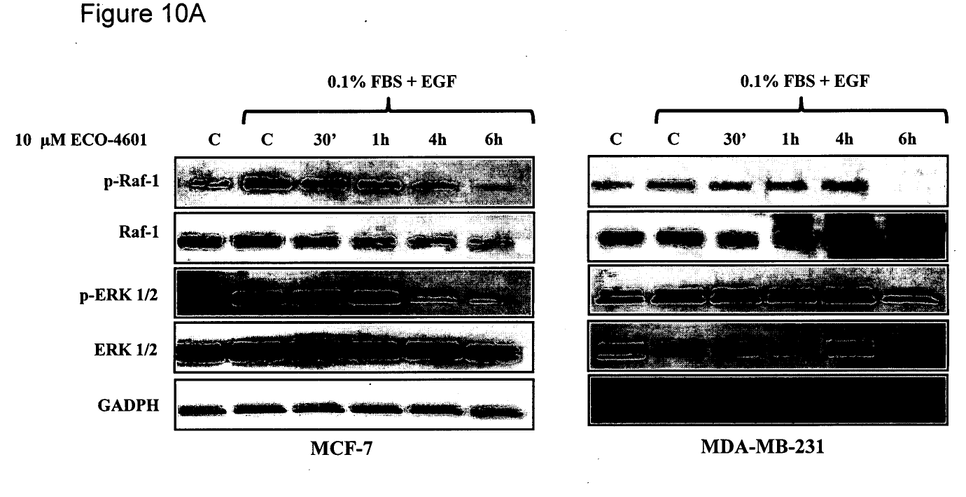

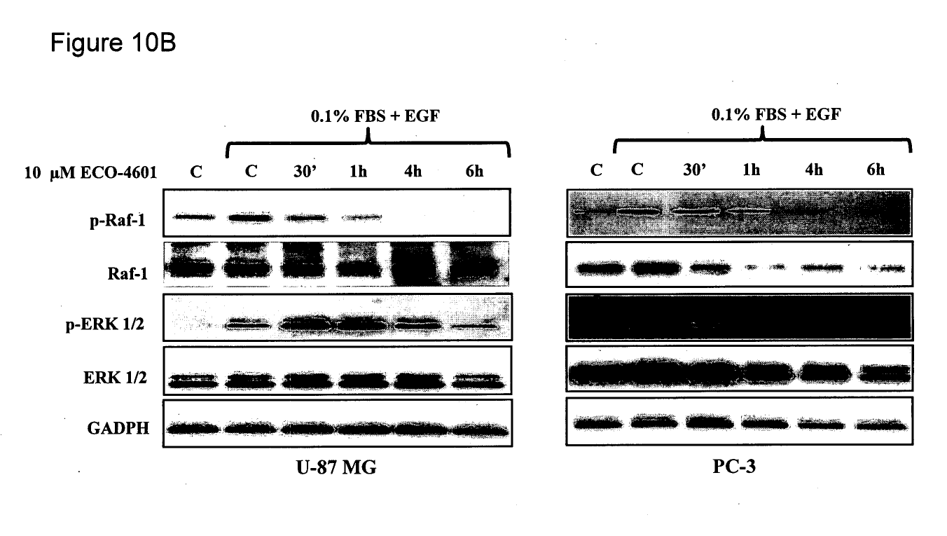

Since the data suggested that ECO-4601 inhibited Ras-MAPK signalling pathway prior to Raf phosphorylation and post Ras prenylation and that the compound was not a direct kinase inhibitor, the effect of ECO-4601 on Ras activation was also evaluated. Following EGF induction, Ras is activated by a nucleotide exchange reaction that removes GDP and replaces it with GTP. Physiological levels of total cellular GTP-bound Ras can be detected with pull-down assays. MCF-7 cells were treated with increasing concentrations of ECO-4601 for 6 h and the Ras-MAPK signalling pathway was then induced with EGF. After a 5 min induction period, cells were lysed and incubated with a recombinant fusion protein that contains the isolated Ras Binding Domain of c-Raf-1 fused the gluthathione-S-transferese (GST; designated GST-Raf-RBD). The presence of Ras in the GST-Raf-RBD protein complex is resolved by western blotting. As expected, after EGF induction, an increase of Ras-GTP was observed. Interestingly, treatment of MCF-7 cells with ECO-4601 prevented EGF from activating Ras (FIG. 10).

Taken together, the in vitro data suggest that ECO-4601 is not a FTI and does not directly inhibit EGFR, c-Raf, MEK1, ERK1 or ERK2 kinase activities. The inhibitory activity of ECO-4601 on the Ras-MAPK signaling pathway was found to be prior to Raf-1 phosphorylation/degradation and post Ras prenylation.

### Example 18

### In Vivo Efficacy of Compound 1 in a K-RAS Mutated Human Tumor

The frequency and type of mutated Ras genes, H-Ras, K-Ras or N-Ras, varies widely depending on the tumor type. K-Ras is the most frequently mutated Ras gene, with a 90% incidence of mutation detected in pancreatic cancer (Downward 2003 Nature Reviews Cancer, 3:11-22; Sebolt-Leopoldd and Herrera 2004 Nature Reviews Cancer 4: 937-947).

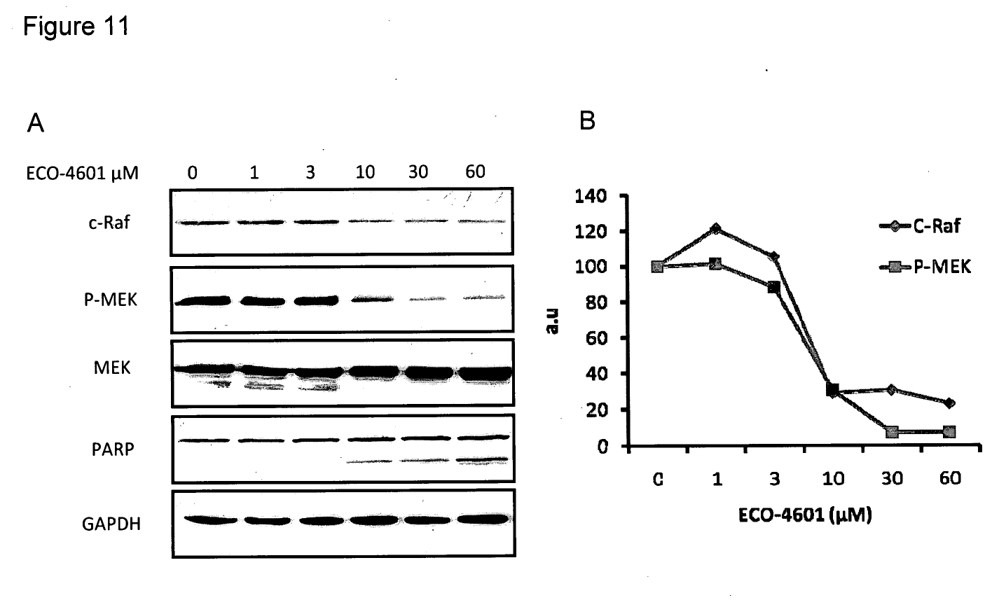

To evaluate whether ECO-4601 interferes with K-Ras signalling, the human pancreatic MiaPaCa-2 cell line, a tumor cell line known to express mutated K-Ras, was employed. ECO-4601 treatment resulted in a dose and time dependent inhibition of EGF-stimulated ERK phosphorylation (FIG. 11).

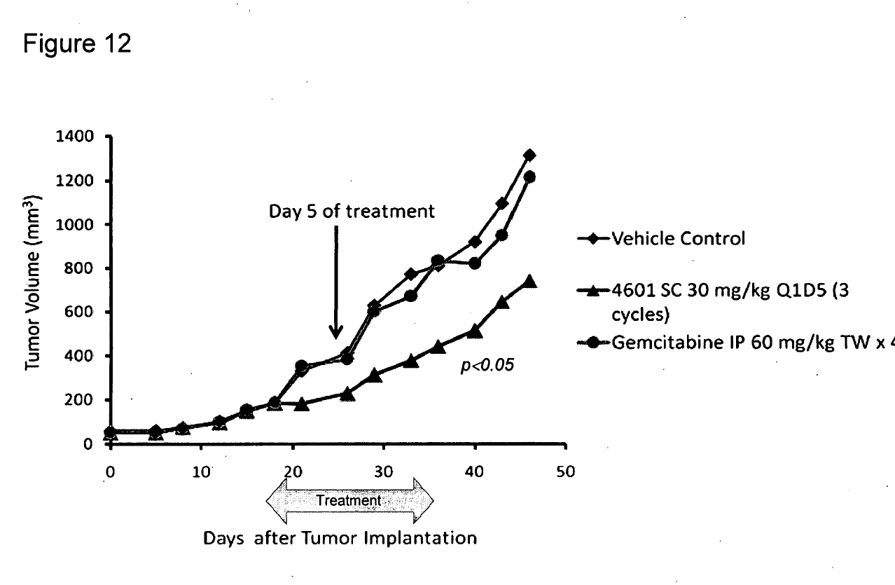

Mice bearing established (100 mm³) human MiaPaCa-2 tumors were dosed with 30 mg/kg (Q1D×5 for 3 wk; SC administration). The positive control group was treated with gemcitabine at 60 mg/kg once a day on Monday and Thursday (TWx4). ECO-4601 as a single agent was statistically different from the vehicle control group (P ˜0.008) following SC daily dosing resulting in a T/C of 56% (FIG. 12).

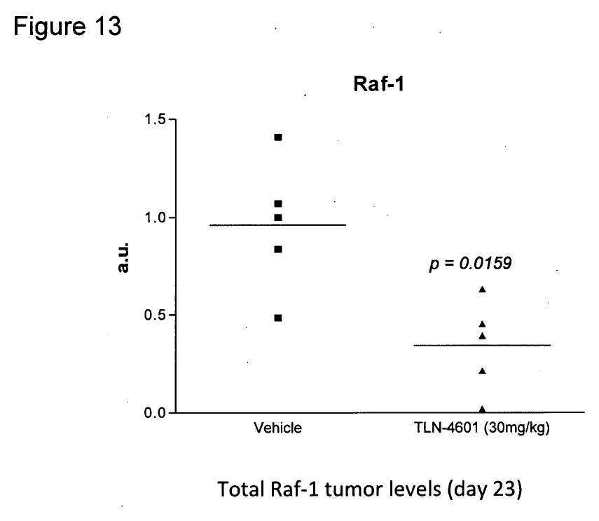

In this model, it was verified if there was an association between antitumor activity and inhibition of the Ras-MAPK pathway. In parallel to the antitumor study described above, two additional groups of 5 mice were treated SC with vehicle or 20 mg/kg ECO-4601, administered daily for 5 days. Animals from these two groups were sacrificed two hours on day 5 of treatment. As shown by quantification of Western blot analysis (FIG. 13), Raf-1 levels were reduced in MiaPaCa-2 tumors obtained from animals treated with ECO-4601 compared with the vehicle-treated animals.

In summary, ECO-4601 was able to inhibit Ras-MAPK signaling of K-ras mutated cells in vivo which correlated with antitumor activity.

### Example 19

### Assays of Dibenzodiazepinone Analogues and Derivatives

### a) Growth Inhibitory Assays:

Growth inhibitory activity of ECO-4601 (Compound 1) and other dibenzodiazepinone analogs was evaluated on a panel of 4 human tumor cell lines: the human uterine sarcoma MES-SA and its doxorubicin-resistant P-glycoprotein over-expressing variant, MES-SA/DX5 as well as non-aggressive and highly aggressive human breast cell lines, MCF-7 and MDA-MB-231, respectively. These four cell lines were obtained from the American Type Culture Collection (Manassas, Va.) and cultured in RPMI plus 10% fetal bovine serum (FBS) and maintained at 37° C. with 5% CO₂.

Exponentially growing cells (5,000 cells per well time; cell number determined with a hemocytometer) were seeded in 96-well flat-bottom plates and allowed to grow overnight. Cells were then incubated for 72 hours with three different concentrations of ECO-4601 or analogs: 30,10, and 3 μM. The in vitro growth inhibitory activity was determined by a commercial MTT assay. All measurements were done in quadruplicate and each experiment was performed 2-3 times. Results are expressed as treated over control and the % of growth inhibition obtained at 10 μM is presented in Table 25. The lower the value, the more cytotoxic is the compound.

TABLE 25

| | % T/C at 10 μM | | | |
|---|---|---|---|---|
| Compounds | MES-SA | MES-SA/DX5 | MCF-7 | MDA-MB-231 |
| ECO-4601 | 78 | 100 | 44 | 38 |
| (Compound 1) | | | | |
| ECO-4625 | 80 | 83 | 28 | 68 |
| (Compound 97) | | | | |
| ECO-4657 | 28 | 24 | 50 | 55 |
| (Compound 99) | | | | |
| 4687 | 18 | 86 | 38 | 43 |
| (Compound 100) | | | | |

The data indicate that ECO-4601 and at least certain analogs of ECO-4601 are potent at inhibiting cell growth. This inhibition occurs in highly aggressive tumor cell lines and for some compounds in cells that are multidrug resistant (MES-SA/5DX).

### (b) PBR Binding Assay

The effect of ECO-4601 and analogs on the peripheral benzodiazepine receptor (PBR) was evaluated in a radioligand-binding assay, implemented in house and described above. The data obtained is presented in Table 26.

TABLE 26

| Compounds | PBR Binding IC₅₀ (μM) |
|---|---|
| 4601 | 2.7 |
| (Compound 1) | |
| 4625 | 2.6 |
| (Compound 97) | |
| 4657 | 0.01 |
| (Compound 99) | |
| 4687 | 0.01 |
| (Compound 100) | |

These data indicate that ECO-4601 and analogs bind the PBR.

### (e) ERKphosphorylation ELISA Assay

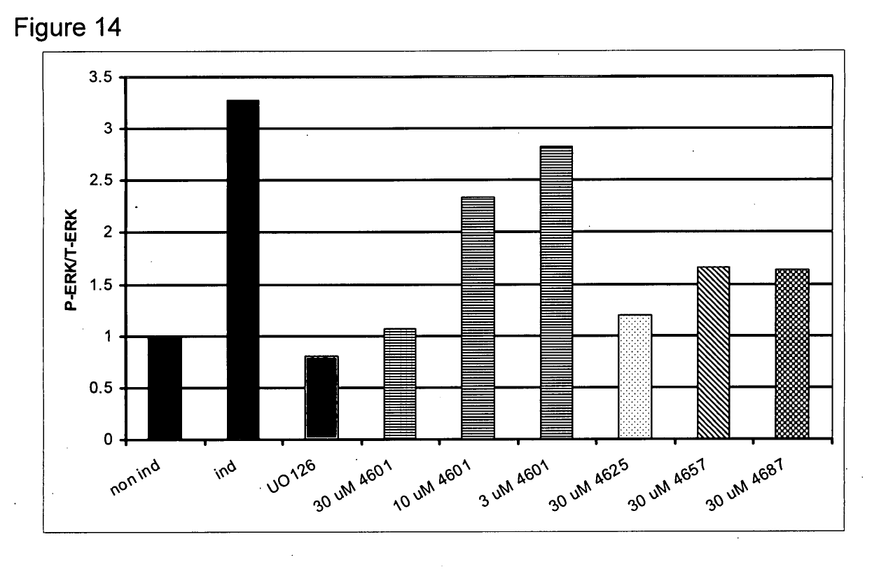

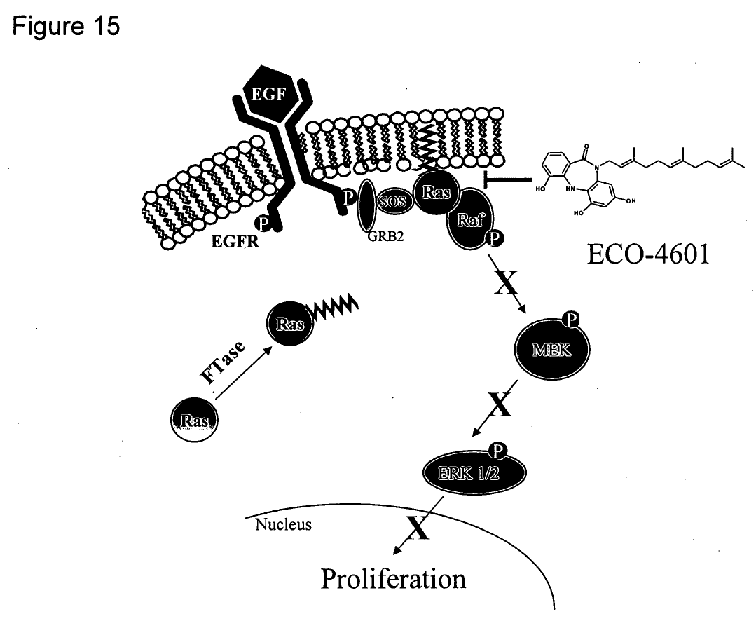

Human breast tumor MCF-7 cells were plated in 96-well culture plates (10,000 cells per well) in RPMI containing 10% FBS. After an overnight incubation, the medium is changed to low serum conditions (RPMI containing 0.1% FBS) for 18 h. Cells were then treated with ECO-4601 or selected analogs for 6 hours and then stimulated by the addition of EGF (100 ng/mL for 5 min) to induce the MAPK pathway. UO126 is a commercial inhibitor (Promega, Madison, Wis.) of mitogen-activated protein kinase kinase (MEK1/ERK). Following stimulation, cells were rapidly fixed, which preserved activation-specific protein modifications. Each well was then incubated with an antibody specific for Phospho-ERK or total ERK. After an one-hour incubation and several washes, cells were incubated with a secondary HRP-conjugated antibody followed by a developing solution that provided a colorimetric readout that is quantitative and reproducible. The Fast Activated Cell-based ELISA (FACE™) is commercially available (Active Motif, Carlsbad, Calif.). The data obtained with Compound 1 and selected analogs clearly demonstrate that they all inhibit the Ras-MAPK signaling pathway shown by their inhibition of phospho-ERK in the FACE ELISA assay (FIG. 14).

From the results obtained in Example 17, a schematic diagram is shown of the Ras-MAPK signaling pathway showing that the inhibitory activity of Compound 1 (ECO-4601) appears to be prior to Raf phosphorylation/degradation.

While this invention has been particularly shown and described with references to preferred embodiments thereof, it will be understood by those skilled in the art that various changes in form and details may be made therein without departing from the spirit of the invention.

All documents, publications, patents, books, manuals, articles, papers and other materials referenced herein are expressly incorporated herein by reference in their entireties.

While this invention has been particularly shown and described with references to preferred embodiments thereof, it will be understood by those skilled in the art that various changes in form and details may be made therein without departing from the

All documents, publications, patents, books, manuals, articles, papers and other materials referenced herein are expressly incorporated herein by reference in their entireties.

What is claimed is:

1. A method for evaluating a chemotherapeutic potential of a candidate molecule, comprising:
(a) determining the ability of a candidate molecule to inhibit the in vitro growth of a cancer cell;
(b) determining the ability of said candidate molecule to bind a cellular receptor produced by a cancer cell, wherein said receptor is produced in a greater amount by said cancer cell than by a normal cell; and
(c) determining the ability of said candidate molecule to inhibit the activity of at least one protein member of the MAPK pathway, thereby evaluating a chemotherapeutic potential of a candidate molecule.

2. The method of claim 1 wherein the candidate molecule is a dibenzodiazepinone analogue or a dibenzodiazepinone analogue derivative of a compound of Formula I 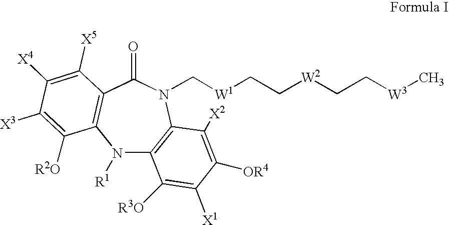
wherein,
W¹, W² and W³ are each independently selected from 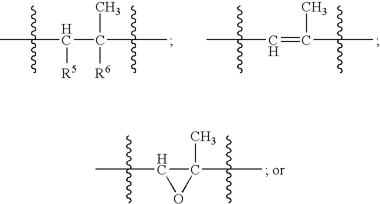
the chain from the tricycle terminates at W³, W² or W¹ with W³, W² or W¹ respectively being either —CH═O, —CH(OC₁₋₆alkyl)₂, —CH₂OH, —CH₂OC₁₋₆alkyl or C(O)OR⁷;
R¹ is selected from the group consisting of H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl, C₃₋₁₀heterocycloalkyl, C(O)H, C(O)C₁₋₁₀alkyl, C(O)C₂₋₁₀alkenyl, C(O)C₂₋₁₀alkynyl, C(O)C₆₋₁₀aryl, C(O)C₅₋₁₀heteroaryl, C(O)C₃₋₁₀cycloalkyl; C(O)C₃₋₁₀heterocycloalkyl and a C-coupled amino acid;
R², R³, and R⁴ are each independently selected from the group consisting of H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl, C₃₋₁₀heterocycloalkyl, C(O)H, C(O)C₁₋₁₀alkyl, C(O)C₂₋₁₀alkenyl, C(O)C₂₋₁₀alkynyl, C(O)C₆₋₁₀aryl, C(O)C₅₋₁₀heteroaryl, C(O)C₃₋₁₀cycloalkyl; C(O)C₃₋₁₀heterocycloalkyl and a C-coupled amino acid;
R⁵ and R⁶ are each independently selected from the group consisting of H, OH, OC₁₋₆alkyl, NH₂, NHC₁₋₆alkyl, N(C₁₋₆alkyl)₂, and NHC(O)C₁₋₆alkyl;
R⁷ is selected from the group consisting of H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl and C₃₋₁₀heterocycloalkyl;
X¹, X², X³, X⁴ and X⁵ are each H; or one of X¹, X², X³, X⁴ or X⁵ is halogen and the remaining ones are H; and
wherein, when any of R¹, R², R³, R⁴, R⁵, R⁶ and R⁷ comprises an alkyl, alkenyl, alkynyl, aryl, heteroaryl, cycloalkyl, or heterocycloalkyl group, then the alkyl, alkenyl, alkynyl, aryl, heteroaryl, cycloalkyl, or heterocycloalkyl group is optionally substituted with substituents selected from the group consisting of acyl, amino, acylamino, acyloxy, carboalkoxy, carboxy, carboxyamido, cyano, halo, hydroxyl, nitro, thio, C₁₋₆alkyl, C₂₋₇alkenyl, C₂₋₇alkynyl, C₃₋₁₀cycloalkyl, C₃₋₁₀heterocycloalkyl, C₆₋₁₀aryl, C₅₋₁₀heteroaryl, alkoxy, aryloxy, sulfinyl, sulfonyl, oxo, guanidino and formyl;
and an ester, ether, N-alkylated or N-acylated derivative, or a pharmaceutically acceptable salt, solvate or prodrug thereof.

3. The method of claim 1 wherein said determining of (b) is determined by a competitive binding assay between said candidate molecule and a cognate ligand of said cellular receptor.

4. The method of claim 1 wherein the receptor is a peripheral benzodiazepinone receptor (PBR).

5. The method of claim 1 wherein the at least one protein member is encoded by a proto-oncogene.

6. The method of claim 1 wherein the at least one protein member is a member of the Ras-MAPK signaling pathway.

7. The method of claim 6 wherein the member of the Ras-MAPK signaling pathway is Ras or Raf.

8. The method of claim 7 wherein the Ras is k-Ras.

9. A method of evaluating a candidate molecule, comprising:
(a) determining the ability of a candidate molecule to bind to a peripheral benzodiazepinone receptor (PBR) with an IC₅₀ (μM) of less than 5; and
(b) determining the ability of said candidate molecule to reduce a signaling activity in the MAPK pathway in a cell upon application of said candidate molecule to said cell in a concentration range of 3 μM to 30 μM, thereby evaluating a candidate molecule.

10. The method of claim 9, wherein said evaluation is an evaluation of anti-cancer activity.

11. The method of claim 9, wherein said candidate molecule is a compound of Formula I 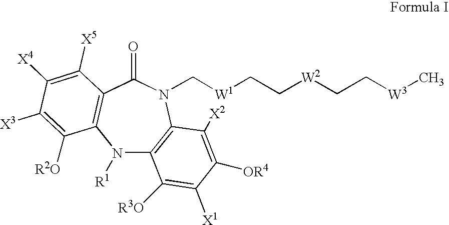
wherein,
W¹, W² and W³ are each independently selected from 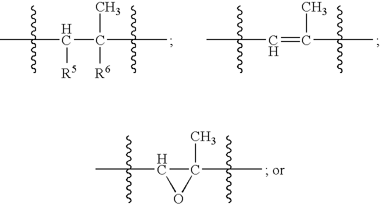
the chain from the tricycle terminates at W³, W² or W¹ with W³, W² or W¹ respectively being either —CH═O, —CH(OC₁₋₆alkyl)₂, —CH₂OH, —CH₂OC₁₋₆alkyl or C(O)OR⁷;
R¹ is selected from the group consisting of H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl, C₃₋₁₀heterocycloalkyl, C(O)H, C(O)C₁₋₁₀alkyl, C(O)C₂₋₁₀alkenyl, C(O)C₂₋₁₀alkynyl, C(O)C₆₋₁₀aryl, C(O)C₅₋₁₀heteroaryl, C(O)C₃₋₁₀cycloalkyl; C(O)C₃₋₁₀heterocycloalkyl and a C-coupled amino acid;
R², R³, and R⁴ are each independently selected from the group consisting of H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl, C₃₋₁₀heterocycloalkyl, C(O)H, C(O)C₁₋₁₀alkyl, C(O)C₂₋₁₀alkenyl, C(O)C₂₋₁₀alkynyl, C(O)C₆₋₁₀aryl, C(O)C₅₋₁₀heteroaryl, C(O)C₃₋₁₀cycloalkyl; C(O)C₃₋₁₀heterocycloalkyl and a C-coupled amino acid;
R⁵ and R⁶ are each independently selected from the group consisting of H, OH, OC₁₋₆alkyl, NH₂, NHC₁₋₆alkyl, N(C₁₋₆alkyl)₂, and NHC(O)C₁₋₆alkyl;
R⁷ is selected from the group consisting of H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl and C₃₋₁₀heterocycloalkyl;
X¹, X², X³, X⁴ and X⁵ are each H; or one of X¹, X², X³, X⁴ or X⁵ is halogen and the remaining ones are H; and
wherein, when any of R¹, R², R³, R⁴, R⁵, R⁶ and R⁷ comprises an alkyl, alkenyl, alkynyl, aryl, heteroaryl, cycloalkyl, or heterocycloalkyl group, then the alkyl, alkenyl, alkynyl, aryl, heteroaryl, cycloalkyl, or heterocycloalkyl group is optionally substituted with substituents selected from the group consisting of acyl, amino, acylamino, acyloxy, carboalkoxy, carboxy, carboxyamido, cyano, halo, hydroxyl, nitro, thio, C₁₋₆alkyl, C₂₋₇alkenyl, C₂₋₇alkynyl, C₃₋₁₀cycloalkyl, C₃₋₁₀heterocycloalkyl, C₆₋₁₀aryl, C₅₋₁₀heteroaryl, alkoxy, aryloxy, sulfinyl, sulfonyl, oxo, guanidino and formyl;
and an ester, ether, N-alkylated or N-acylated derivative, or a pharmaceutically acceptable salt, solvate or prodrug thereof.

12. The method of claim 9, wherein said signaling activity is reduced by inhibiting an activity of a Ras MAPK protein family member.

13. The method of claim 12, wherein the Ras MAPK protein family member is Ras or Raf.

14. A compound of the formula: 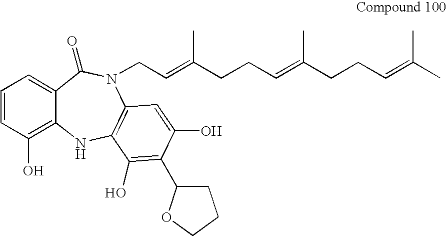
or a pharmaceutically acceptable salt thereof.

15. A pharmaceutical composition comprising a compound selected from the group comprising: 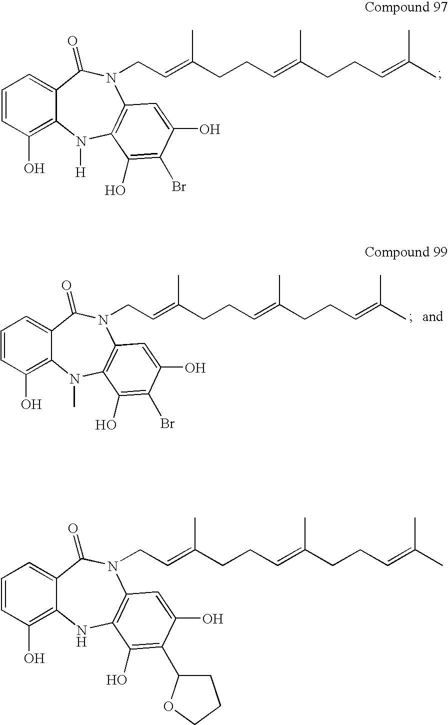
or a pharmaceutically acceptable salt thereof, together with a pharmaceutically acceptable carrier.

16. A method for lessening a non-specific cytotoxic side-effect of an anti-cancer treatment administered to a mammal, comprising administrating to a mammal having cancer and being treated with an anti-cancer treatment a molecule having each of activities (a), (b) and (c) in accordance with the method of claim 1.

17. The method of claim 16, wherein said molecule is not Compound 1

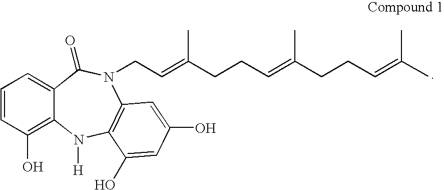

18. A method for lessening a non-specific cytotoxic side-effect of an anti-cancer treatment administered to a mammal, comprising administrating to a mammal having cancer and being treated with an anti-cancer treatment a molecule having each of activities (a) and (b) in accordance with the method of claim 9.

19. The method of claim 18, wherein said molecule is not Compound 1

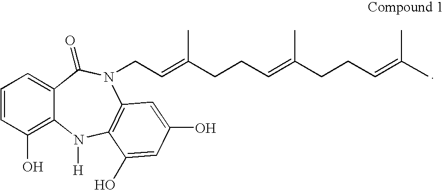

20. A compound identified by the method of claim 1 having each of activities (a), (b) and (c).

21. A compound identified by the method of claim 9 having each of activities (a) and (b).
